(19) United States
(12) Patent Application Publication
(10) Pub. No.: US 2012/0134993 A1
(43) Pub. Date: May 31, 2012

(54) COMPOSITIONS AND METHODS FOR USING MULTISPECIFIC-BINDING PROTEINS COMPRISING AN ANTIBODY-RECEPTOR COMBINATION

(75) Inventors: Qi Pan, Needham, MA (US); Carl W. Birks, Seattle, WA (US); Pallavur V. Sivakumar, Seattle, WA (US)

(73) Assignee: ZymoGenetics, Inc.

(21) Appl. No.: 13/260,408

(22) PCT Filed: Mar. 26, 2010
(86) PCT No.: PCT/US10/28877

§ 371 Date: Nov. 7, 2011

Related U.S. Application Data
(60) Provisional application No. 61/164,023, filed on Mar. 27, 2009.

(51) Int. Cl. A61K 39/395; C12N 15/63; C12P 21/02; A61P 35/00; C12N 5/10; C12N 1/19; C12N 1/15; C07K 19/00; C12N 15/62

(52) U.S. Cl. 424/134.1; 530/387.3; 435/320.1; 435/69.6; 536/23.4; 435/328; 435/419; 435/254.21; 435/254.23; 435/254.2; 435/254.22; 435/254.3; 435/254.4; 435/254.11; 435/252.33; 435/252.31

(57) ABSTRACT

Disclosed are bispecific binding proteins comprising a antibody/soluble receptor bispecific binding protein that reduces the biological activity of both VEGF-A and FGF. The FGF binding moieties are generally soluble FGFR3 or FGFR2. An Fc polypeptide is fused to the C-terminus of the FGF binding moiety and VEGF-A binding moiety are polypeptides fused using peptide or polypeptide linker sequences, and can be expressed as single bispecific binding protein. The bispecific antibody/soluble receptor binding proteins can be used to treat cancers characterized by solid tumor growth as well as other diseases.

### BACKGROUND OF THE INVENTION

Angiogenesis is the formation of new blood vessels from existing vessels. It plays an essential role during development. In adults, angiogenesis occurs during wound healing to restore blood flow to tissues after injury or insult. Angiogenesis also plays an important role in tumor formation and in other diseases, including rheumatoid arthritis, atherosclerosis, psoriasis, diabetic retinopathy, and macular degeneration. (See, e.g., Fan et al., Trends Pharmacol. Sci. 16:57, 1995; Folkman, Nature Med. 1:27, 1995.)

The growth of new blood vessels under physiological or pathological conditions requires the concerted action of activators and inhibitors of angiogenesis. Activators of angiogenesis include vascular endothelial growth factor-A (VEGF-A), fibroblast growth factors (FGFs), placenta growth factor (P1GF), and hepatocyte growth factor (HGF) and some cytokines such as interleukin-8 (IL-8). Endogenous inhibitors of angiogenesis include thrombospondin, endostatin, angiostatin and interleukin-12. The balance between activators and inhibitors of angiogenesis is tilted towards activators during physiological and pathological angiogenesis.

VEGF-A is a key regulator of both physiological and pathological angiogenesis. It plays an essential role in the specification, morphogenesis, differentiation and homeostasis of vessels by regulating the proliferation, migration, and survival of endothelial cells. (See, e.g., Ferrara et al., Nat Med 9:669, 2003.) Studies indicated that VEGF-A is highly expressed in a variety of human tumors. (See, e.g., Ellis and Hicklin, Nat. Rev. Cancer 8:579, 2008.) VEGF-A expression is regulated by the hypoxia-inducible factor 1 (HIF-1) transcription factor. (See, e.g., Wang and Semenza, J. Biol. Chem. 270:1230, 1995.) Rapid proliferation of tumor cells and poor blood flow resulted in a hypoxia-conductive environment in tumors, leading to rapid upregulatin of VEGF-A. (See, e.g., Brahimi-Horn and Pouyssegur, Bull. Cancer 93:E73, 2006.)

Five human VEGF-A isoforms of 121, 145, 165, 189 or 206 amino acids in length (VEGF-A₁₂₁₋₂₀₆), encoded by distinct mRNA splice variants, have been described, all of which are capable of stimulating mitogenesis in endothelial cells. These isoforms differ in biological activity, receptor specificity, and affinity for cell surface- and extracellular matrix-associated heparan-sulfate proteoglycans, which behave as low affinity receptors for VEGF-A: VEGF-A₁₂₁ does not bind to either heparin or heparan-sulfate; VEGF-A₁₄₅ and VEGF-A₁₆₅ (GenBank Acc. No. M32977) are both capable of binding to heparin; and VEGF-A₁₈₉ and VEGF-A₂₀₆ show the strongest affinity for heparin and heparan-sulfates. VEGF-A₁₂₁, VEGF-A₁₄₅, and VEGF-A₁₆₅ are secreted in a soluble form, although most of VEGF-A₁₆₅ is confined to cell surface and extracellular matrix proteoglycans, whereas VEGF-A₁₈₉ and VEGF-A₂₀₆ remain associated with extracellular matrix. Both VEGF-A₁₈₉ and VEGF-A₂₀₆ can be released by treatment with heparin or heparinase, indicating that these isoforms are bound to extracellular matrix via proteoglycans. Cell-bound VEGF-A₁₈₉ can also be cleaved by proteases such as plasmin, resulting in release of an active soluble VEGF-A₁₁₀. Human VEGF-A₁₆₅, the most abundant and biologically active form, is glycosylated at Asn74 and is typically expressed as a 46 kDa homodimer of 23 kDa subunits.

Four cell-surface receptors that interact with VEGF-A have been identified. These include VEGFR-1/Flt-1 (fins-like tyrosine kinase-1; GenBank Acc. No. X51602; De Vries et al., Science 255:989-991, 1992); VEGFR-2/KDR/Flk-1 (kinase insert domain containing receptor/fetal liver kinase-1; GenBank Acc. Nos. X59397 (Flk-1) and L04947 (KDR); Terman et al., Biochem. Biophys. Res. Comm. 187:1579-1586, 1992; Matthews et al., Proc. Natl. Acad. Sci. USA 88:9026-9030, 1991); neuropilin-1 (Gen Bank Acc. No. NM003873), and neuropilin-2 (Gen Bank Acc. No. NM003872). VEGF₁₂₁ and VEGF₁₆₅ bind VEGFR-1; VEGF₁₂₁, VEGF₁₄₅, and VEGF₁₆₅ bind VEGFR-2; VEGF₁₆₅ binds neuropilin-1; and VEGF₁₆₅ and VEGF₁₄₅ bind neuropilin-2. (See, e.g., Neufeld et al., FASEB J. 13:9-22, 1999; Stacker and Achen, Growth Factors 17:1-11, 1999; Ortega et al., Fron. Biosci. 4:141-152, 1999; Zachary, Intl. J. Biochem. Cell Bio. 30:1169-1174, 1998; Petrova et al., Exp. Cell Res. 253:117-130, 1999.)

Recognition of the importance of VEGF-A for the development of several important classes of cancer recently culminated in the approval of AVASTIN™, a humanized monoclonal antibody to VEGF-A, for combination treatment with chemotherapy for metastatic colorectal cancer, nonsmall cell lung cancer and metastatic breast cancer. (See, e.g., Hervitz et al., N. Engl. J. Med. 350:2335-2342, 2004; Sandler et al., N. Engl. J. Med. 355:2542-2550, 2006; Miller et al., 2008). Similarly, the importance of VEGF-A in the pathogenesis of neovascular ocular disorders is reflected in the recent approval of LUCENTIS™, a humanized monoclonal antibody fragment, for the treatment of neovascular (wet) age-related macular degeneration (AMD).

Fibroblast growth factors (FGFs) are a family of heparin-binding growth factors with 22 family members in mammals (FGF1-14, 16-23). FGFs play important roles in a variety of biological functions such as cell proliferation, differentiation, migration, angiogenesis and tumorigenesis. They execute their pleiotropic biological actions by binding, dimerizing and activating cell surface FGF receptors. (See, e.g., Eswarakumar et al. Cytokine Growth Factor Rev. 16:139-149, 2005.) There are four FGF receptor genes in mammals, fgfR1-fgfR4. The extracellular domain of FGFRs comprises three immunoglobulin-like domains. Alternative splicing at the membrane proximal Ig loop of FgfR1-FgfR3 give rise to additional variants. This loop is encoded by an invariant exon (IIIa), for the N-terminal half, and a choice of exons termed IIIb or IIIc for the other half.

Overexpression of FGF ligands and receptors and mutants in FGF receptors have been associated with many types of cancer, including prostate, breast, ovarian, bladder, colorectal, pancreatic, liver, lung, glioblastoma cancers, multiple myeloma and leukemia. (See e.g., Grose et al., Cytokine Growth Factor Rev. 16:179-186, 2005). FGF1, 2, 6, 8b, 9 and 17 are over-expressed in prostate tumor tissues, and the expression levels of FGF8b and 17 are correlated with tumor stage, grade and poor prognosis (Dorkin et al., Oncogene 18:2755-2761, 1999; Gnanapragasam et al., Oncogene 21:5069-5080, 2002; Heer et al., J. Pathol. 204:578-586. 2004). FGF9 contributes to prostate cancer-induced new bone formation and may participate in the osteoblastic progression of androgen receptor-negative prostate cancer in bone (Li et al., J Clin Invest. 118:2697-2710, 2008). FGFR1 and FGFR4 are over-expressed in prostate tumor tissues, and FGFR2IIIb to IIIc isoform switch promotes prostate cancer initiation and progression (Giri et al., Clin Cancer Res. 5:1063-1071, 1999; Wang et al., Clin. Cancer Res. 10:6169-6178, 2004; Kwabi-Addo et al., Prostate 46:163-172, 2001). FGF1, 2, 8 are over-expressed in breast tumor tissues. Up to 8.7% of all breast cancers have FGFR1 gene amplication and this amplification is an independent predictor of overall survival. FGFR4 overexpression correlates with fail on tamoxifen therapy in patients with recurrent breast cancer (See, e.g., Elsheikh et al., Breast Cancer Res. 9, 2007; Meijer et al., Endocrine-Related Cancer 15:101-111, 2008). FGF1, 8, 9, 18 and FGFR1_{III}c, FGFR2_{III}c, FGFR4 are over-expressed in ovarian tumor tissues. FGFR3 over-expression and activating mutations have been reported in urothelial cell carcinoma of bladder cancer. FGFR3 mutation in non-invasive, low-grade and stage bladder tumors significantly associate with higher recurrence rate. (See, e.g., Knowles, World J. Urol. 25:581-593, 2007.) FGF-2, FGFR1 and FGFR2 are frequently over-expressed in squamous cell carcinoma and adenocarcinoma of the lung. FGF-2 signaling pathway activation may be an early phenomenon in the pathogenesis of squamous cell carcinoma (Behrens, et. al., Clin Cancer Res. 14:6014-6022, 2008).

Many members of the FGF family, including FGF1, FGF2, FGF4 and FGF6, also have strong pro-angiogenic activity in vitro and in vivo, and can promote tumor progression by modulating tumor vascularization (Presta et al., Cytokine Growth Factor Rev. 16:159-178, 2005). An intimate cross-talk exists between members of the FGF family and the VEGF family during angiogenesis. VEGF blockade with an anti-VEGFR2 monoclonal antibody promotes hypoxia and induces the expression of FGF1, FGF2 and FGF7 in tumor tissues in the Rip1-Tag2 transgenic mice that develop spontaneous pancreatic tumors (Casanovas et al., Cancer Cell 8:299-309, 2005). The upregulation of FGFs co-incides with the reinduction of angiogenesis and escape from VEGF blockade. Combined inhibition of VEGF and FGF signaling in this model results in further tumor suppression, demonstrating that upregulation of the FGF signaling pathway contributes at least partially to escape mechanisms after VEGF-targeted therapy. Furthermore, blocking VEGF and FGF signaling in several mouse tumor models, including the T3M4, Panc1 and QG56 xenograft models has shown additive or synergistic anti-tumor effects (Ogawa et al., Cancer Gene Ther. 9:633-640, 2002). Recently, a clinical study of glioblastoma patients treated with a pan-VEGFR tyrosine kinase inhibitor shows that serum levels of FGF2 are higher in relapsing patients than in that of the same patients during the response phase, indicating a similar compensation mechanism involving upregulation of FGF2 (Batchelor et al., Cancer Cell 11:83-95, 2007).

Taken together, the above preclinical and clinical data support the idea that a combination treatment blocking both the VEGF and the FGF signaling pathways could produce a better anti-tumor effect in many solid tumors than VEGF blockade alone. These data provide strong proof-of-concept rationale for targeting both pathways in oncology. Blocking these two pathways together may also provide better efficacy in other angiogenesis diseases, including AMD. The present invention provides multispecific proteins for these and other uses that will be apparent to those skilled in art from the teachings herein.

### SUMMARY OF THE INVENTION

The present invention provides bispecific binding proteins comprising a antibody/soluble receptor bispecific binding protein that reduces the biological activity of both VEGF-A and FGF. In accordance with the present invention, the bispecific binding protein comprises a VEGF-A binding region of an anti-VEGF-A antibody (VEGF-A antibody) moiety and a FGF binding moiety of an FGF receptor, as described herein. The FGF binding moieties described here are generally soluble FGF receptors (FGFR). The invention provides that in certain embodiments the soluble FGF receptor portion of the bispecific binding protein comprises an FGF receptor moiety of an FGFR3 or FGFR2 as described herein. In other embodiments, an Fc polypeptide is fused to the C-terminus of the FGFR. In certain embodiments, the FGF binding moiety and VEGF-A binding moiety are polypeptides fused using peptide or polypeptide linker sequences, and in these instances the polynucleotides encoding said embodiments can be expressed as single bispecific binding protein.

The invention also provides that certain embodiments of the bispecific binding protein comprises a VEGF-A antibody moiety as described herein. The VEGF-A antibody moiety can further be comprised of scFV polypeptides or VL and VH polypeptides described herein.

In certain embodiments the FGF binding moiety is an FGF receptor moiety, and can be FGFR3, and in particular is FGFR3_{IIIc} as described herein. In certain embodiments, a bispecific antibody/soluble receptor protein comprises an FGF receptor moiety that is an FGFR3 selected from the group consisting of FGFR3_{IIIc}(23-375) as shown in SEQ ID NO:13, FGFR3_{IIIc}(23-375)(S249W) as shown in SEQ ID NO:2, FGFR3_{IIIc}(143-375) as shown in SEQ ID NO:19, FGFR3_{IIIc}(143-375)(S249W), as shown in SEQ ID NO:10, FGFR3_{IIIc}(23-375)(P250R) as shown in SEQ ID NO:15, and FGFR3_{IIIc}(143-375)(P250R) as shown in SEQ ID NO:22 in combination with a VEGF-A antibody moiety selected from the group consisting of c870.1e6 scFV as shown in SEQ ID NO:44, c1094.1 scFV as shown in SEQ ID NO:46, c870 scFV as shown in SEQ ID NO:52, and c1039 scFV as shown in SEQ ID NO:70. In other embodiments, a bispecific antibody/soluble receptor combination comprises an FGF binding moiety that is an FGFR3 selected from the group consisting of FGFR3_{IIIc}(23-375) as shown in SEQ ID NO:13, FGFR3_{IIIc}(23-375)(S249W) as shown in SEQ ID NO:2, FGFR3_{IIIc}(143-375) as shown in SEQ ID NO:19, FGFR3_{IIIc}(143-375)(S249W), as shown in SEQ ID NO:10, FGFR3_{IIIc}(23-375)(P250R) as shown in SEQ ID NO:15, and FGFR3_{IIIc}(143-375)(P250R) as shown in SEQ ID NO:22 and VEGF-A binding moiety selected from the group consisting of a c870 VL as shown in SEQ ID NO:48 and VH as shown in SEQ ID NO:50, a c1094 VL as shown in SEQ ID NO:54 and VH as shown in SEQ ID NO:56, and a 1039 VL as shown in SEQ ID NO:66 and VH as shown in SEQ ID NO:68.

In other embodiments, the bispecific binding protein of the present invention embodies an FGFR3 moiety and VEGF-A antibody moiety selected from the group consisting of FGFR3(143-375)(S249W)Fc5 c1094.1 pZMP31 (SEQ ID NO:58); FGFR3(23-375)(S249W)Fc5 c1094.1 pZMP31 (SEQ ID NO:60); FGFR3(143-375)(S249W)Fc5 c870e6 pZMP31 (SEQ ID NO:62); and FGFR3(23-375)(S249W)Fc5 c870e6 pZMP31 (SEQ ID NO:64).

In other embodiments the FGF binding moiety is FGFR2. In certain embodiments the FGFR2 comprises FGFR2_{IIIc}. In certain embodiments, a bispecific antibody/soluble receptor combinations comprises an FGF binding moiety that is an FGFR2 selected from the group consisting of FGFR2_{IIIc}(22-377) as shown in SEQ ID NO:24, FGFR2_{IIIc}(22-377)(S252W) as shown in SEQ ID NO:29, FGFR2_{IIIc}(22-377)(P253R) as shown in SEQ ID NO:33, FGFR2_{IIIc}(145-377), as shown in SEQ ID NO:37, FGFR2_{IIIc}(145-377)(S252W) as shown in SEQ ID NO:40, and FGFR2_{IIIc}(145-377)(P253R) as shown in SEQ ID NO:42; and VEGF-A binding moiety selected from the group consisting of c870.1e6 scFV as shown in SEQ ID NO:44, c1094.1 scFV as shown in SEQ ID NO:46, c870 scFV as shown in SEQ ID NO:52, and c1039 scFV as shown in SEQ ID NO:70. In other embodiments, a bispecific antibody/soluble receptor combination comprises an FGF binding moiety that is an FGFR₂ selected from the group consisting of FGFR_{IIIc}(22-377) as shown in SEQ ID NO:24, FGFR_{IIIc}(22-377)(S252W) as shown in SEQ ID NO:29, FGFR_{2IIIc}(22-377)(P253R) as shown in SEQ ID NO:33, FGFR_{2IIIc}(145-377), as shown in SEQ ID NO:37, FGFR_{2IIIc}(145-377)(S252W) as shown in SEQ ID NO:40, and FGFR_{2IIIc}(145-377)(P253R) as shown in SEQ ID NO:42; and VEGF-A binding moiety selected from the group consisting a c870 VL as shown in SEQ ID NO:48 and VH as shown in SEQ ID NO:50, a c1094 VL as shown in SEQ ID NO:54 and VH as shown in SEQ ID NO:56, and a 1039 VL as shown in SEQ ID NO:66 and VH as shown in SEQ ID NO:68.

In other aspects, the present invention provides for methods of using the bispecific antibody/soluble receptor binding proteins described herein. In certain embodiments, the bispecific antibody/soluble receptor binding proteins can be administered to a subject to treat cancers characterized by solid tumor growth such as prostate cancer, breast cancer, pancreatic cancer, renal cell carcinoma (RCC), colorectal cancer, glioblastoma, non-small cell lung cancer (NSCLC), and gastrointestinal stromal tumor (GIST).

These and other aspects of the invention will become evident upon reference to the following detailed description of the invention and the attached drawings.

### DEFINITIONS

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

A “polypeptide” is a polymer of amino acid residues joined by peptide bonds, whether produced naturally or synthetically. Polypeptides of less than about 10 amino acid residues are commonly referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used herein, the term “antagonist” denotes a compound that reduces the activity of another compound in a biological setting. Thus, a VEGF-A antagonist is a compound that reduces the biological activity of VEGF-A, and a FGFR antagonist is compound that reduces the biological activity of FGF. Since the activities of both VEGF-A and FGF are dependent on the interactions of multiple molecules (including ligand, receptor, and signal transducers), antagonists can reduce the activity by acting directly on VEGF-A or FGF, or by acting on another molecule in the cognate biological pathway. For example, a FGF antagonist can reduce FGF activity by, e.g., binding to the receptor itself, by binding to one of its ligands, by interfering with receptor dimerization, or by interfering with receptor phosphorylation. Antagonists include, without limitation, antibodies, soluble receptors, and non-proteinaceous compounds that bind to a ligand or its receptor, or otherwise interfering with ligand-receptor interactions and/or other receptor functions.

The term “receptor” denotes a cell-associated protein that binds to a bioactive molecule (i.e., a ligand) and mediates the effect of the ligand on the cell. Membrane-bound receptors are characterized by a multi-domain or multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, soluble or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

A “soluble receptor” is a receptor polypeptide that is not bound to a cell membrane. Soluble receptors are most commonly ligand-binding receptor polypeptides that lack transmembrane and cytoplasmic domains. Soluble receptors can comprise additional amino acid residues, such as affinity tags that provide for purification of the polypeptide or provide sites for attachment of the polypeptide to a substrate. Many cell-surface receptors have naturally occurring, soluble counterparts that are produced by proteolysis or translated from alternatively spliced mRNAs. Receptor polypeptides are said to be substantially free of transmembrane and intracellular polypeptide segments when they lack sufficient portions of these segments to provide membrane anchoring or signal transduction, respectively.

As used herein, the term “Fc-fusion protein” designates antibody-like molecules which combine the binding specificity of a heterologous protein with the effector functions of immunoglobulin constant domains. Structurally, the Fc-fusion proteins comprise a fusion of an amino acid sequence with the desired binding specificity which is other than the antigen recognition and binding site of an antibody (i.e., is “heterologous”), and an immunoglobulin constant domain sequence. The Fc-fusion protein molecule typically includes a contiguous amino acid sequence comprising at least the binding site of a receptor or a ligand. The immunoglobulin constant domain sequence in the Fc-fusion protein can be obtained from any immunoglobulin, such as IgG-1, IgG-2, IgG-3, or IgG-4 subtypes, IgA (including IgA-1 and IgA-2), IgE, IgD or IgM. For example, useful Fc-fusion proteins according to this invention are polypeptides that comprise the FGF binding portions of a FGFR3 receptor without the transmembrane or cytoplasmic sequences of the FGFR3 receptor. In one embodiment, the extracellular domain of FGFR3 is fused to a constant domain of an immunoglobulin sequence.

The term “antibody” is used herein to denote proteins produced by the body in response to the presence of an antigen and that bind to the antigen, as well as antigen-binding fragments and engineered variants thereof. Hence, the terms “antibody” and “antibodies” include polyclonal antibodies, affinity-purified polyclonal antibodies, monoclonal antibodies, and antigen-binding antibody fragments, such as F(ab′)₂ and Fab fragments. Genetically engineered intact antibodies and fragments, such as chimeric antibodies, humanized antibodies, single-chain Fv fragments, single-chain antibodies, diabodies, minibodies, linear antibodies, multivalent or multispecific hybrid antibodies, and the like are also included. Thus, the term “antibody” is used expansively to include any protein that comprises an antigen binding site of an antibody and is capable of binding to its antigen.

The term “genetically engineered antibodies” means antibodies wherein the amino acid sequence has been varied from that of a native antibody. Because of the relevance of recombinant DNA techniques in the generation of antibodies, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with cells and other effector functions. Typically, changes in the variable region will be made in order to improve the antigen binding characteristics, improve variable region stability, or reduce the risk of immunogenicity.

An “antigen-binding site of an antibody” is that portion of an antibody that is sufficient to bind to its antigen. The minimum such region is typically a variable domain or a genetically engineered variant thereof. Single-domain binding sites can be generated from camelid antibodies (see Muyldermans and Lauwereys, J. Mol. Recog. 12:131-140, 1999; Nguyen et al., EMBO J. 19:921-930, 2000) or from V_{H} domains of other species to produce single-domain antibodies (“dAbs”; see Ward et al., Nature 341:544-546, 1989; U.S. Pat. No. 6,248,516 to Winter et al.). In certain variations, an antigen-binding site is a polypeptide region having only 2 complementarity determining regions (CDRs) of a naturally or non-naturally (e.g., mutagenized) occurring heavy chain variable domain or light chain variable domain, or combination thereof (see, e.g., Pessi et al., Nature 362:367-369, 1993; Qiu et al., Nature Biotechnol. 25:921-929, 2007). More commonly, an antigen-binding site of an antibody comprises both a heavy chain variable domain and a light chain variable domain that bind to a common epitope. Within the present invention, a molecule that “comprises an antigen-binding site of an antibody” may further comprise one or more of a second antigen-binding site of an antibody (which may bind to the same or a different epitope or to the same or a different antigen), a peptide linker, an immunoglobulin constant domain, an immunoglobulin hinge, an amphipathic helix (see Pack and Pluckthun, Biochem. 31:1579-1584, 1992), a non-peptide linker, an oligonucleotide (see Chaudri et al., FEBS Letters 450:23-26, 1999), and the like, and may be a monomeric or multimeric protein. Examples of molecules comprising an antigen-binding site of an antibody are known in the art and include, for example, Fv fragments, single-chain Fv fragments (scFv), Fab fragments, diabodies, minibodies, Fab-scFv fusions, bispecific (scFv)₄-IgG, and bispecific (scFv)₂-Fab. (See, e.g., Hu et al., Cancer Res. 56:3055-3061, 1996; Atwell et al., Molecular Immunology 33:1301-1312, 1996; Carter and Merchant, Curr. Opin. Biotechnol. 8:449-454, 1997; Zuo et al., Protein Engineering 13:361-367, 2000; and Lu et al., J. Immunol. Methods 267:213-226, 2002.)

As used herein, the term “immunoglobulin” refers to a protein consisting of one or more polypeptides substantially encoded by immunoglobulin gene(s). One form of immunoglobulin constitutes the basic structural unit of an antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. Immunoglobulins typically function as antibodies in a vertebrate organism. Five classes of immunoglobulin protein (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol γ. For example, immunoglobulins of the IgG1 subclass contain a γ1 heavy chain constant region. Each immunoglobulin heavy chain possesses a constant region that consists of constant region protein domains (C_{H}1, hinge, C_{H}2, and C_{H}3; IgG3 also contains a C_{H}4 domain) that are essentially invariant for a given subclass in a species. DNA sequences encoding human and non-human immunoglobulin chains are known in the art. (See, e.g., Ellison et al., DNA 1:11-18, 1981; Ellison et al., Nucleic Acids Res. 10:4071-4079, 1982; Kenten et al., Proc. Natl. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

Full-length immunoglobulin “light chains” (about 25 Kd or 214 amino acids) are encoded by a variable region gene at the NH₂-terminus (encoding about 110 amino acids) and a by a kappa or lambda constant region gene at the COOH-terminus. Full-length immunoglobulin “heavy chains” (about 50 Kd or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin “Fv” fragment contains a heavy chain variable domain (V_{H}) and a light chain variable domain (V_{L}), which are held together by non-covalent interactions. An immunoglobulin Fv fragment thus contains a single antigen-binding site. The dimeric structure of an Fv fragment can be further stabilized by the introduction of a disulfide bond via mutagenesis. (See Almog et al., Proteins 31:128-138, 1998.)

As used herein, the terms “single-chain Fv” and “single-chain antibody” refer to antibody fragments that comprise, within a single polypeptide chain, the variable regions from both heavy and light chains, but lack constant regions. In general, a single-chain antibody further comprises a polypeptide linker between the V_{H} and V_{L} domains, which enables it to form the desired structure that allows for antigen binding. Single-chain antibodies are discussed in detail by, for example, Pluckthun in The Pharmacology of Monoclonal Antibodies, vol. 113 (Rosenburg and Moore eds., Springer-Verlag, New York, 1994), pp. 269-315. (See also WIPO Publication WO 88/01649; U.S. Pat. Nos. 4,946,778 and 5,260,203; Bird et al., Science 242:423-426, 1988.) Single-chain antibodies can also be bi-specific and/or humanized.

A “Fab fragment” contains one light chain and the C_{H}1 and variable regions of one heavy chain. The heavy chain of a Fab fragment cannot form a disulfide bond with another heavy chain molecule.

A “Fab′ fragment” contains one light chain and one heavy chain that contains more of the constant region, between the C_{H}1 and C_{H}2 domains, such that an interchain disulfide bond can be formed between two heavy chains to form a F(ab′)₂ molecule.

A “F(ab′)₂ fragment” contains two light chains and two heavy chains containing a portion of the constant region between the C_{H}1 and C_{H}2 domains, such that an interchain disulfide bond is formed between two heavy chains.

An immunoglobulin “Fc fragment” (or Fc domain) is the portion of an antibody that is responsible for binding to antibody receptors on cells and the C1q component of complement. Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, C_{H}2, and C_{H}3 domains. However, more recently the term has been applied to a single chain consisting of C_{H}3, C_{H}2, and at least a portion of the hinge sufficient to form a disulfide-linked dimer with a second such chain. For a review of immunoglobulin structure and function, see Putnam, The Plasma Proteins, Vol. V (Academic Press, Inc., 1987), pp. 49-140; and Padlan, Mol. Immunol. 31:169-217, 1994. As used herein, the term Fc includes variants of naturally occurring sequences.

An immunoglobulin light or heavy chain variable region consists of a “framework” region interrupted by three hypervariable regions. Thus, the term “hypervariable region” refers to the amino acid residues of an antibody that are responsible for antigen binding. The hypervariable region comprises amino acid residues from a “Complementarity Determining Region” or “CDR” (e.g., in human, residues 24-34 (L1), 50-56 (L2), and 89-97 (L3) in the light chain variable domain and residues 31-35 (H1), 50-65 (H2) and 95-102 (H3) in the heavy chain variable domain (amino acid sequence numbers based on the EU index; see Kabat et al., Sequences of Proteins of Immunological Interest, 5th Ed. Public Health Service, National Institutes of Health, Bethesda, Md. (1991)) and/or those residues from a “hypervariable loop” (in human, residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, J. Mol. Biol. 196: 901-917, 1987) (both of which are incorporated herein by reference). “Framework Region” or “FR” residues are those variable domain residues other than the hypervariable region residues as herein defined. The sequences of the framework regions of different light or heavy chains are relatively conserved within a species. Thus, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDR's. The CDR's are primarily responsible for binding to an epitope of an antigen. CDRs L1, L2, and L3 of the V_{L} domain are also referred to herein, respectively, as LCDR1, LCDR2, and LCDR3; CDRs H1, H2, and H3 of the V_{H} domain are also referred to herein, respectively, as HCDR1, HCDR2, and HCDR3.

“Chimeric antibodies” are antibodies whose light and heavy chain genes have been constructed, typically by genetic engineering, from immunoglobulin variable and constant region genes belonging to different species. For example, the variable segments of the genes from a mouse monoclonal antibody may be joined to human constant region-encoding segments (e.g., human gamma 1 or gamma 3 heavy chain genes, and human kappa light chain genes). A therapeutic chimeric antibody is thus a hybrid protein, typically composed of the variable or antigen-binding domains from a mouse antibody and the constant domains from a human antibody, although other mammalian species may be used. Specifically, a chimeric antibody is produced by recombinant DNA technology in which all or part of the hinge and constant regions of an immunoglobulin light chain, heavy chain, or both, have been substituted for the corresponding regions from another animal's immunoglobulin light chain or heavy chain. In this way, the antigen-binding portion of the parent monoclonal antibody is grafted onto the backbone of another species' antibody. Chimeric antibodies may be optionally “cloaked” with a human-like surface by replacement of exposed residues, the result of which is a “veneered antibody.”

As used herein, the term “human antibody” includes an antibody that has an amino acid sequence of a human immunoglobulin and includes antibodies isolated from human immunoglobulin libraries or from animals transgenic for one or more human immunoglobulin genes and that do not express endogenous immunoglobulins, as described, for example, in U.S. Pat. No. 5,939,598 to Kucherlapati et al.

The term “humanized immunoglobulin” refers to an immunoglobulin comprising a human framework region and one or more CDR's from a non-human (e.g., a mouse or rat) immunoglobulin. The non-human immunoglobulin providing the CDR's is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDR's, are substantially identical to corresponding parts of natural human immunoglobulin sequences. In some instances, humanized antibodies may retain non-human residues within the human variable region framework domains to enhance proper binding characteristics (e.g., mutations in the frameworks may be required to preserve binding affinity when an antibody is humanized). A “humanized antibody” is an antibody comprising a humanized light chain and a humanized heavy chain immunoglobulin. For example, a humanized antibody would not encompass a typical chimeric antibody as defined above because, e.g., the entire variable region of a chimeric antibody is non-human.

A “bispecific antibody” or “bifunctional antibody” is a hybrid antibody having two different heavy/light chain pairs and two different binding sites. Bispecific antibodies may be produced by a variety of methods including, but not limited to, fusion of hybridomas or linking of Fab′ fragments. See, e.g., Songsivilai & Lachmann, Clin. Exp. Immunol. 79:315-321, 1990; Kostelny et al., J. Immunol. 148:1547-1553, 1992.

A “bivalent antibody” other than a “multispecific” or “multifunctional” antibody, in certain embodiments, is an antibody comprising two binding sites having identical antigenic specificity.

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448, 1993.

The term “minibody” refers herein to a polypeptide that encodes only 2 complementarity determining regions (CDRs) of a naturally or non-naturally (e.g., mutagenized) occurring heavy chain variable domain or light chain variable domain, or combination thereof. Examples of minibodies are described by, e.g., Pessi et al., Nature 362:367-369, 1993; and Qiu et al., Nature Biotechnol. 25:921-929, 2007.

The term “linear antibodies” refers to the antibodies described in Zapata et al., Protein Eng. 8:1057-1062, 1995. Briefly, these antibodies comprise a pair of tandem Fd segments (V_{H}-C_{H1}-V_{H}-C_{H1}) which form a pair of antigen binding regions. Linear antibodies can be bispecific or monospecific.

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

The term “parent antibody” as used herein refers to an antibody which is encoded by an amino acid sequence used for the preparation of the variant. Preferably, the parent antibody has a human framework region and, if present, has human antibody constant region(s). For example, the parent antibody may be a humanized or human antibody.

A “variant” anti-VEGF-A antibody, refers herein to a molecule which differs in amino acid sequence from a “parent” anti-VEGF-A antibody amino acid sequence by virtue of addition, deletion and/or substitution of one or more amino acid residue(s) in the parent antibody sequence. In the preferred embodiment, the variant comprises one or more amino acid substitution(s) in one or more hypervariable region(s) of the parent antibody. For example, the variant may comprise at least one, e.g., from about one to about ten, and preferably from about two to about five, substitutions in one or more hypervariable regions of the parent antibody. Ordinarily, the variant will have an amino acid sequence having at least 75% amino acid sequence identity with the parent antibody heavy or light chain variable domain sequences, more preferably at least 80%, more preferably at least 85%, more preferably at least 90%, and most preferably at least 95%. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind human VEGF-A and preferably has properties which are superior to those of the parent receptor or antibody. For example, the variant may have a stronger binding affinity, enhanced ability to inhibit VEGF-A-induced biological activity (e.g., angiogenesis or proliferation). To analyze such properties, one should compare a Fab form of the variant to a Fab form of the parent antibody or a full length form of the variant to a full length form of the parent antibody, for example, since it has been found that the format of an anti-VEGF-A antibody impacts its activity in the biological activity assays disclosed herein. The variant antibody of particular interest herein is one which displays about at least a 3 fold, 5 fold, 10 fold, 20 fold, or 50 fold, enhancement in biological activity when compared to the parent antibody.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. More specifically, the term “VEGF-A epitope” as used herein refers to a portion of the VEGF-A polypeptide having antigenic or immunogenic activity in an animal, preferably in a mammal, and most preferably in a mouse or a human. An epitope having immunogenic activity is a portion of a VEGF-A polypeptide that elicits an antibody response in an animal. An epitope having antigenic activity is a portion of a VEGF-A polypeptide to which an antibody immunospecifically binds as determined by any method well known in the art, for example, by immunoassays. Antigenic epitopes need not necessarily be immunogenic.

A “vector” is a nucleic acid molecule, such as a plasmid, cosmid, or bacteriophage, that has the capability of replicating autonomously in a host cell. Cloning vectors typically contain one or a small number of restriction endonuclease recognition sites that allow insertion of a nucleic acid molecule in a determinable fashion without loss of an essential biological function of the vector, as well as nucleotide sequences encoding a marker gene that is suitable for use in the identification and selection of cells transformed with the cloning vector. Marker genes typically include genes that provide tetracycline resistance or ampicillin resistance.

An “expression vector” is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked” to the promoter. Similarly, a regulatory element and a core promoter are operably linked if the regulatory element modulates the activity of the core promoter.

The term “expression” refers to the biosynthesis of a gene product. For example, in the case of a structural gene, expression involves transcription of the structural gene into mRNA and the translation of mRNA into one or more polypeptides.

With regard to proteins as described herein, reference to amino acid residues corresponding to those specified by SEQ ID NO includes post-translational modifications of such residues.

The terms “neovascularization” and “angiogenesis” are used interchangeably herein. Neovascularization and angiogenesis refer to the generation of new blood vessels into cells, tissue, or organs. The control of angiogenesis is typically is typically altered in certain disease states and, in many case, the pathological damage associated with the disease is related to altered or unregulated angiogenesis. Persistent, unregulated angiogenesis occurs in a variety of disease states, including those characterized by the abnormal growth by endothelial cells, and supports the pathological damage seen in these conditions including leakage and permeability of blood vessels.

The term “neovascular disorder” are used herein refers to any disease or disorder having a pathology that is mediated, at least in part, by increased or unregulated angiogenesis activity. Examples of such diseases or disorders include various cancers comprising solid tumors (e.g., pancreatic cancer, renal cell carcinoma (RCC), colorectal cancer, non-small cell lung cancer (NSCLC), and gastrointestinal stromal tumor (GIST)) as well as certain ocular diseases involving neovascularization (“neovascular ocular disorders”). Such diseases or disorders are particularly amenable to certain treatment methods for inhibition angiogenesis, as described further herein.

The term “effective amount,” in the context of treatment of a neovascular disorder by administration of a FGFR and/or VEGF-A antagonist to a subject as described herein, refers to an amount of such agent that is sufficient to inhibit angiogenesis in the subject so as to inhibit the occurrence or ameliorate one or more symptoms of the neovascular disorder. An effective amount of an agent is administered according to the methods of the present invention in an “effective regime.” The term “effective regime” refers to a combination of amount of the agent being administered and dosage frequency adequate to accomplish treatment or prevention of the disease or disorder.

The term “patient” or “subject,” in the context of treating a disease or disorder as described herein, includes mammals such as, for example, humans and other primates. The term also includes domesticated animals such as, e.g., cows, hogs, sheep, horses, dogs, and cats.

Two amino acid sequences have “100% amino acid sequence identity” if the amino acid residues of the two amino acid sequences are the same when aligned for maximal correspondence. Similarly, two nucleotide sequences have “100% nucleotide sequence identity” if the nucleotide residues of the two nucleotide sequences are the same when aligned for maximal correspondence. Sequence comparisons can be performed using standard software programs such as those included in the LASERGENE bioinformatics computing suite, which is produced by DNASTAR (Madison, Wis.). Other methods for comparing two nucleotide or amino acid sequences by determining optimal alignment are well-known to those of skill in the art. (See, e.g., Peruski and Peruski, The Internet and the New Biology: Tools for Genomic and Molecular Research (ASM Press, Inc. 1997); Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two nucleotide or amino acid sequences are considered to have “substantially similar sequence identity” or “substantial sequence identity” if the two sequences have at least 80%, at least 90%, or at least 95% sequence identity relative to each other.

Percent sequence identity is determined by conventional methods. See, e.g., Altschul et al., Bull. Math. Bio. 48:603, 1986, and Henikoff and Henikoff, Proc. Natl. Acad. Sci. USA 89:10915, 1992. For example, two amino acid sequences can be aligned to optimize the alignment scores using a gap opening penalty of 10, a gap extension penalty of 1, and the “BLOSUM62” scoring matrix of Henikoff and Henikoff, supra, as shown in Table 1 (amino acids are indicated by the standard one-letter codes). The percent identity is then calculated as:

([Total number of identical matches]/[length of the longer sequence plus the number of gaps introduced into the longer sequence in order to align the two sequences])(100).

TABLE 1
BLOSUM62 Scoring Matrix

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and a second amino acid sequence. The FASTA algorithm is described by Pearson and Lipman, Proc. Nat'l Acad. Sci. USA 85:2444, 1988, and by Pearson, Meth. Enzymol. 183:63, 1990. Briefly, FASTA first characterizes sequence similarity by identifying regions shared by the query sequence (e.g., residues 25-266 of SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63, 1990.

FASTA can also be used to determine the sequence identity of nucleic acid molecules using a ratio as disclosed above. For nucleotide sequence comparisons, the ktup value can range between one to six, preferably from three to six, most preferably three, with other parameters set as described above.

### BRIEF DESCRIPTION OF THE FIGURES

FIGS. 1A-1C illustrate the amino acid sequences of certain immunoglobulin Fc polypeptides. Amino acid sequence numbers are based on the EU index (Kabat et al., Sequences of Proteins of Immunological Interest, US Department of Health and Human Services, NIH, Bethesda, 1991). The illustrated sequences include a wild-type human sequence (“wt”; SEQ ID NO:75) and five variant sequences, designated Fc-488 (SEQ ID NO:76), Fc4 (SEQ ID NO:77), Fc5 (SEQ ID NO:74), Fc6 (SEQ ID NO:78), and Fc7 (SEQ ID NO:79). The Cys residues normally involved in disulfide bonding to the light chain constant region (LC) and heavy chain constant region (HC) are indicated. A “.” indicates identity to wild-type at that position. *** indicates the stop codon; the C-terminal Lys residue has been removed from Fc6. Boundaries of the hinge, C_{H}2, and C_{H}3 domains are shown.

FIG. 2 depicts a tetravalent, bispecific antibody/soluble receptor combination with specificity for two different targets (referred to herein as αVEGF-A and ligand binding domain of FGFR).

FIG. 3 depicts FGFR-Fc (R&D Systems) showing variable inhibition of FGF-9-stimulated proliferation of osteoblasts.

FIG. 4A depicts the inhibition of FGF-9-stimulated proliferation by FGFR-Fc constructs (ZymoGenetics) Full-length FGFR3-Fc wild-type and mutant constructs (ZymoGenetics) have similar IC50s and FIG. 4B depicts Truncated FGFR3-Fc mutant constructs (ZymoGenetics) have similar IC50s.

FIG. 5A depicts the direct binding of FGF-8b by FGFR-Fc (R&D Systems) and FIG. 5B depicts the direct binding of FGF-8b by FGFR-Fc constructs (ZymoGenetics).

FIG. 6A depicts the direct binding of FGF-17 by FGFR-Fc (R&D Systems) and FIG. 6B depicts the direct binding of FGF-17 by FGFR2-Fc constructs (ZymoGenetics).

FIG. 7 depicts the direct binding of FGF-17 by FGFR3-Fc constructs (ZymoGenetics).

FIG. 8A depicts FGFR-Fc inhibits growth of Caki-1 cells and FIG. 8B depicts FGFR-Fc inhibits growth of DU145 cells.

FIG. 9 depicts the second and third Ig-like domains of the FGF receptor family.

### DESCRIPTION OF THE INVENTION

### I. Overview

The present invention addresses a need in the art to provide more therapeutics to treat cancers, particularly solid tumors, by providing new proteins that are multispecific binding proteins, in particular bispecific binding proteins. The proteins that comprise a soluble receptor moiety fused to an antibody moiety. As used herein, the term “bispecific binding protein” refers to a protein capable of specifically binding to at least two different target molecules via at least two binding moieties having different binding specificities. The binding moieties may be, for example, a protein (e.g., antibody or soluble receptor) or small molecule. The binding moieties of a bispecific binding protein may be physically linked. The present invention as described herein, provides bispecific binding proteins which comprise a soluble receptor moiety and an antibody moiety.

In the present invention the soluble receptor moiety comprises a soluble FGF receptor or portion thereof and the antibody moiety comprises a VEGF-A antibody or portion thereof as described herein. In certain embodiments, two or more different moieties of a bispecific binding protein are linked via linker to form a multimer (e.g., a dimer). For example, in the case of a bispecific binding protein comprising a fusion of at least two polypeptide moieties (e.g., soluble FGF receptor and a VEGF-A antibody), a peptide linker sequence may be employed to separate, for example, the polypeptide components by a distance sufficient to ensure that each polypeptide folds into its secondary and tertiary structures.

In certain embodiments, a bispecific binding protein of the invention reduces the biological activity of both FGF and VEGF-A. Specifically, the present invention provides VEGF-A and FGF antagonists, particularly neutralizing anti-VEGF-A antibodies in combination with FGF soluble receptors, that reduce signaling through VEGF-A receptors and FGF receptors. Reduction of angiogenic signals through VEGF-A and/or FGF using such antagonists are useful for treatment of various disorders having a pathology characterized at least in part by neovascularization. For example, inhibition of angiogenic signals through VEGF-A and/or FGF in and around tumors reduces the tumor's ability to vascularize, grow, and metastasize.

The activation of FGF receptors can activate multiple signal transduction pathways including the phospholipase C, phosphatidyl inositol 3-kinase, mitogen-activated protein kinase and signal transducers and activators of transcription (STAT) pathways, all of which play a role in prostate cancer progression. The net result of increased FGF signaling includes enhanced proliferation, resistance to cell death, increased motility and invasiveness, increased angiogenesis, enhanced metastasis, resistance to chemotherapy and radiation and androgen independence, all of which can enhance tumor progression and clinical aggressiveness. FGF receptors and/or FGF signaling can affect both the tumor cells directly and tumor angiogenesis (Kwabi-Addo et al., Endocrine-Related Cancer 11 (4) 709-724, 2004).

The present invention provides VEGF-A and FGF antagonists that reduce the biological activity of both VEGF-A and FGF. The FGF-binding moiety is a soluble FGF receptor (FGFR) and the VEGF-A-binding moiety is a VEGF-A antibody. In accordance with the present invention the VEGF-A and FGF antagonists are bispecific antibody/soluble receptor binding proteins that specifically bind to and reduce VEGF-A and FGF activity. Bispecific binding proteins of the invention are described in detail herein.

### II. FGF Receptors, Anti-VEGF-A Antibodies, and Related Bispecific Binding Compositions

A. FGF receptors

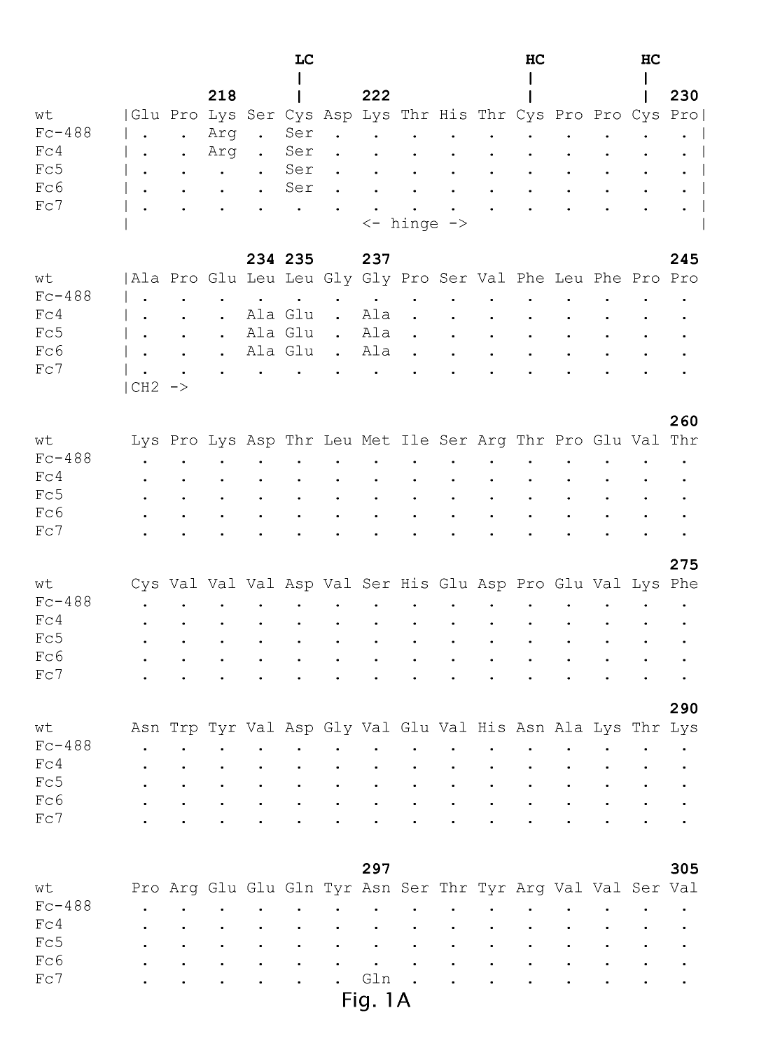

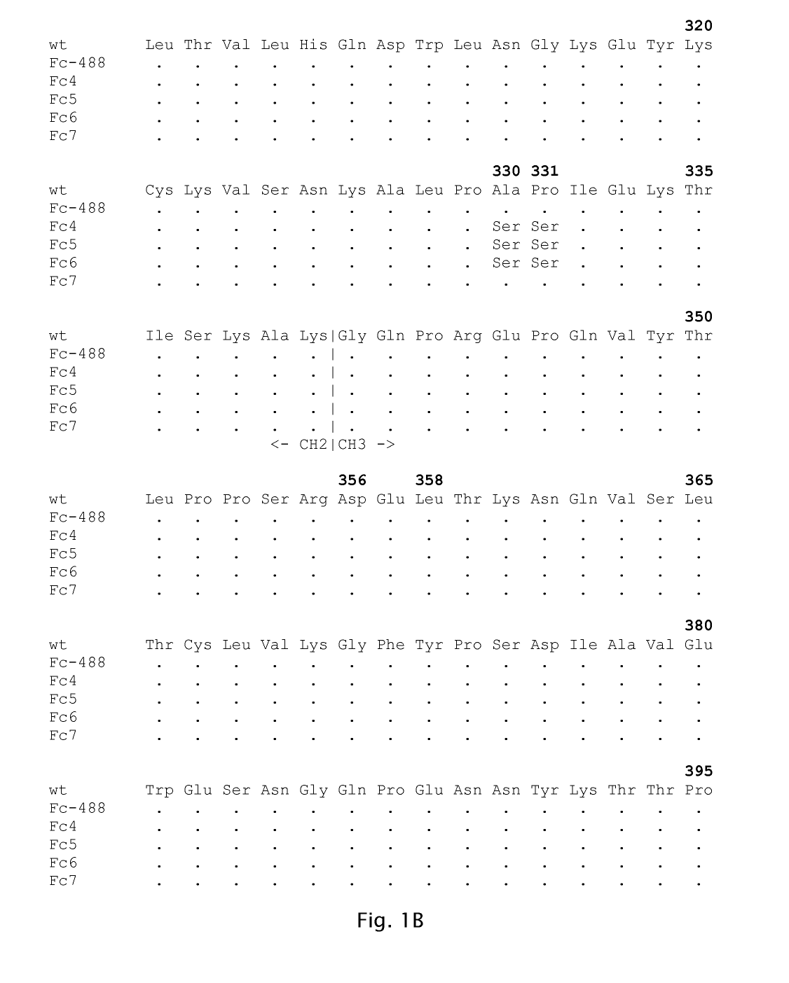

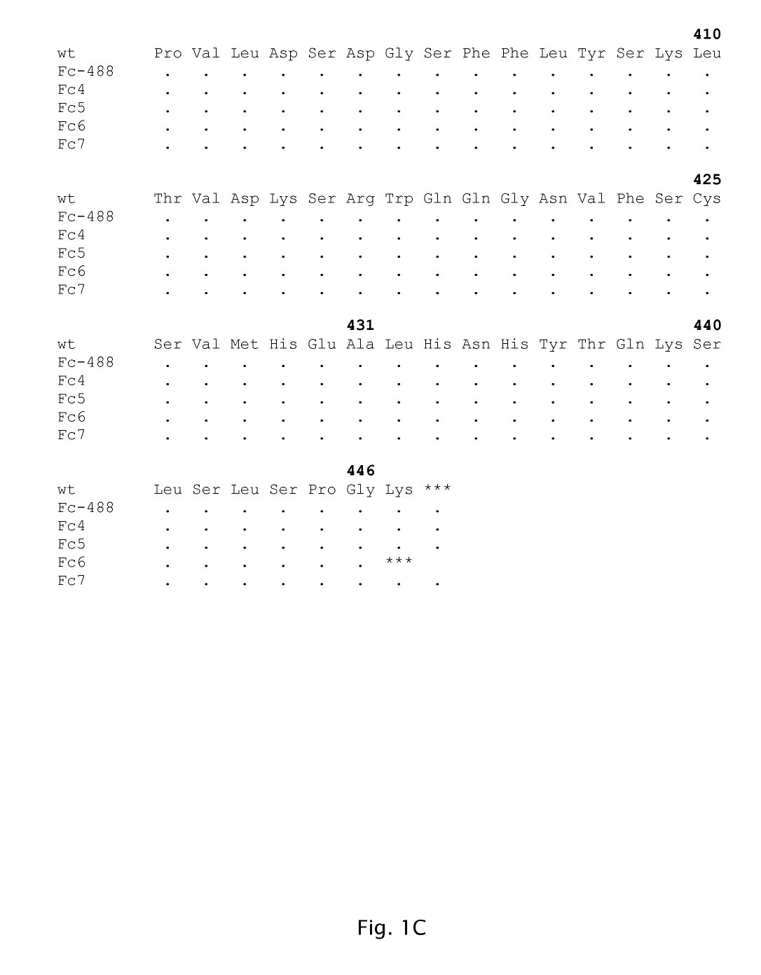

The FGFR portion of the molecule is a soluble receptor. The FGFR comprises three Ig-like domains referred to as D1, D2 and D3. The receptor can comprise D1, D2, D3 or can comprise D2, D3 without D1 of the FGF receptor. Furthermore, the receptor may be the native receptor or with mutations in the D2-D3 region. The FGFR family and domains D2 and D3 are shown in FIG. 1.

It has been demonstrated that truncation of D1 can increase the affinity of the receptor and enhance the receptor/ligand interaction (Olsen et al. PNAS 101:935-940, 2004). FGFR1-3 are known to have isoforms as a result of alternative splicing at the carboxyterminal of D3. Beginning with this knowledge, the present inventors generated multiple variant soluble FGF receptors with three or two Ig-like domains and characterized their binding affinity for FGF ligands. The FGFR3 and FGFR2 isoforms IIIc are demonstrated to be particularly interesting because they have relatively higher affinity for FGF 2, 6, 8b, 9 and 17, than the corresponding IIb isoforms.

FGF receptors can be characterized by their binding affinity for FGF ligands. Association rate constants (kₐ (M⁻¹s⁻¹)) and dissociation rate constants (k_{d} (s⁻¹)) are measured for a given interaction. The association rate constant is a value that reflects the rate of the ligand-receptor complex formation. The dissociation rate constant is a value that reflects the stability of this complex. Equilibrium binding affinity is typically expressed as either an equilibrium dissociation constant (K_{D} (M)) or an equilibrium association constant (K_{A} (M⁻¹)). K_{D} is obtained by dividing the dissociation rate constant by the association rate constant (k_{d}/kₐ), while K_{A} is obtained by dividing the association rate constant by the dissociation rate constant (kₐ/k_{d}). Molecules with similar K_{D} (or a similar K_{A}) can have widely variable association and dissociation rate constants. Binding affinities for the bispecific binding proteins of the present invention will be in the range of 100 nM or less, preferably 10 nM or less, and more preferably 1 nM or less when measured in a standard in vitro assay such as in BIACORE binding analyses.

In certain embodiments, the FGFR is FGFR3III_{c}, Other specific embodiments include FGFR3III_{c} where amino acid number 262 of SEQ ID NO:2 or amino acid number 142 of SEQ ID NO:9 was mutated from S to W. Other specific embodiments include FGFR3III_{c} where amino acid number 263 of SEQ ID NO:15 or amino acid number 143 of SEQ ID NO:22 was mutated from P to R. In other embodiments, the FGFR3III_{c} may be truncated at the N-terminal as shown in SEQ ID NOS:10, 19, and 22.

In other certain embodiments, the FGFR is FGFR2III_{c}, Other specific embodiments include FGFR2III_{c} where amino acid number X of SEQ ID NO:29 or amino acid number Xa of SEQ ID NO:40 was mutated from S to W. Other specific embodiments include FGFR2III_{c} where amino acid number Y of SEQ ID NO:33 or amino acid number Ya of SEQ ID NO:42 was mutated from P to R. In other embodiments, the FGFR2III_{c} may be truncated at the N-terminal as shown in SEQ ID NOS:37 and 42.

B. VEGF-A Antibodies

VEGF-A antagonists for use within the present invention include molecules that bind to VEGF-A or a VEGF-A receptor and thereby reduce the activity of VEGF-A on cells that express the receptor such as, e.g., VEGFR-1, VEGFR-2, neuropilin-1, and/or neuropilin-2. In particular, VEGF-A antagonists include anti-VEGF-A antibodies. Other suitable VEGF-A antagonists include soluble VEGF-A receptors comprising a VEGFR extracellular domain, as well as small molecule antagonists capable of inhibiting the interaction of VEGF-A with its receptor or otherwise capable in inhibiting VEGF-A-induced intracellular signaling through a VEGF-A receptor. In addition, binding proteins based on non-antibody scaffolds may be employed. (See, e.g., Koide et al., J. Mol. Biol. 284:1141-1151, 1998; Hosse et al. Protein Sci. 15:14-27, 2006, and references therein.) Preferred VEGF-A antagonists for use within the invention include antibodies that specifically bind to VEGF-A, including bispecific antibodies that also comprise a binding site for FGF. Antibodies that are specific for VEGF-A bind at least the soluble secreted forms of VEGF-A, and preferably also bind cell surface-associated forms.

Antibodies are considered to be specifically binding if (1) they exhibit a threshold level of binding activity, and (2) they do not significantly cross-react with control polypeptide molecules. For example, a threshold level of binding is determined if an anti-VEGF-A antibody binds to a VEGF-A polypeptide, peptide or epitope with an affinity at least 10-fold greater than the binding affinity to a control (non-VEGF-A) polypeptide. It is preferred that antibodies used within the invention exhibit a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, commonly by surface plasmon resonance using automated equipment. Other methods are known in the art, for example Scatchard analysis (Scatchard, Ann. NY Acad. Sci. 51:660-672, 1949).

Antibodies of the present invention comprise or consist of portions of intact antibodies that retain antigen-binding specificity. Suitable antibodies include, for example, fully human antibodies; humanized antibodies; chimeric antibodies; antibody fragments such as, e.g., Fab, Fab′, F(ab)₂, F(ab′)₂ and Fv antibody fragments; single chain antibodies; and monomers or dimers of antibody heavy or light chains or mixtures thereof. Preferred antibodies of the invention are monoclonal antibodies. Antibodies comprising a light chain may comprise kappa or lambda light chain.

In certain embodiments, antibodies of the invention include intact immunoglobulins of any isotype including IgA, IgG, IgE, IgD, or IgM (including subtypes thereof). Intact immunoglobulins in accordance with the present invention preferably include intact IgG (e.g., intact IgG1, IgG2, IgG3, IgG4, IgA1, or IgA2).

Methods for preparing and isolating polyclonal antibodies, monoclonal antibodies, and antigen-binding antibody fragments thereof are well known in the art. See, e.g., Current Protocols in Immunology, (Cooligan et al. eds., John Wiley and Sons, Inc. 2006); Sambrook et al., Molecular Cloning: A Laboratory Manual (Cold Spring Harbor, N.Y., 2nd ed. 1989); and Monoclonal Hybridoma Antibodies: Techniques and Applications (Hurrell ed., CRC Press, Inc., Boca Raton, Fla., 1982). Antigen binding fragments, including scFv, can be prepared using phage display libraries according to methods known in the art. Phage display can also be employed for the preparation of binding proteins based on non-antibody scaffolds (Koide et al., supra.). Methods for preparing recombinant human polyclonal antibodies are disclosed by Wiberg et al., Biotechnol Bioeng. 94:396-405, 2006; Meijer et al., J. Mol. Biol. 358:764-772, 2006; Haurum et al., U.S. Patent Application Publication No. 2002/0009453; and Haurum et al., U.S. Patent Application Publication No. 2005/0180967.

As would be evident to one of ordinary skill in the art, polyclonal antibodies for use within the present invention can be generated by inoculating any of a variety of warm-blooded animals such as horses, cows, goats, sheep, dogs, chickens, rabbits, mice, and rats with an immunogenic polypeptide or polypeptide fragment. The immunogenicity of an immunogenic polypeptide can be increased through the use of an adjuvant, such as alum (aluminum hydroxide) or Freund's complete or incomplete adjuvant. Polypeptides useful for immunization also include fusion polypeptides, such as fusions of VEGF-A or a portion thereof with an immunoglobulin polypeptide or with maltose binding protein. The polypeptide immunogen may be a full-length molecule or a portion thereof. If the polypeptide portion is hapten-like, it may be advantageously joined or linked to a macromolecular carrier (such as keyhole limpet hemocyanin (KLH), bovine serum albumin (BSA) or tetanus toxoid) for immunization.

In addition, antibodies can be screened against known polypeptides related to the antibody target (e.g., orthologs, paralogs, or sequence variants of, for example, to isolate a population of antibodies that is highly specific for binding to the target protein or polypeptide. Such highly specific populations include, for example, antibodies that bind to human VEGF-A but not to mouse VEGF-A. Such a lack of cross-reactivity with related polypeptide molecules is shown, for example, by the antibody detecting a VEGF-A polypeptide but not known, related polypeptides using a standard Western blot analysis (Current Protocols in Molecular Biology (Ausubel et al. eds., Green and Wiley and Sons, NY 1993)) or ELISA (enzyme immunoassay) (Immunoassay, A Practical Guide (Chan ed., Academic Press, Inc. 1987)). In another example, antibodies raised to a VEGF-A polypeptide are adsorbed to related polypeptides adhered to insoluble matrix; antibodies that are highly specific to the VEGF-A polypeptide will flow through the matrix under the proper buffer conditions. Screening allows isolation of polyclonal and monoclonal antibodies non-cross-reactive to known, closely related polypeptides (Antibodies: A Laboratory Manual (Harlow and Lane eds., Cold Spring Harbor Laboratory Press 1988); Current Protocols in Immunology (Cooligan et al. eds., National Institutes of Health, John Wiley and Sons, Inc. 1995). Screening and isolation of specific antibodies is well known in the art. See Fundamental Immunology (Paul ed., Raven Press 1993); Getzoff et al., Adv. in Immunol. 43:1-98, 1988; Monoclonal Antibodies: Principles and Practice (Goding ed., Academic Press Ltd. 1996); Benjamin et al., Ann. Rev. Immunol. 2:67-101, 1984.

Native monoclonal antibodies (“mAbs”) can be prepared, for example, by immunizing subject animals (e.g., rats or mice) with a purified immunogenic protein or fragment thereof. In a typical procedure, animals are each given an initial intraperitoneal (IP) injection of the purified protein or fragment, typically in combination with an adjuvant (e.g., Complete Freund's Adjuvant or RIBI Adjuvant (available from Sigma-Aldrich, St. Louis, Mo.)) followed by booster IP injections of the purified protein at, for example, two-week intervals. Seven to ten days after the administration of the third booster injection, the animals are bled and the serum is collected. Additional boosts can be given as necessary. Splenocytes and lymphatic node cells are harvested from high-titer animals and fused to myeloma cells (e.g., mouse SP2/0 or Ag8 cells) using conventional methods. The fusion mixture is then cultured on a feeder layer of thymocytes or cultured with appropriate medium supplements (including commercially available supplements such as Hybridoma Fusion and Cloning Supplement; Roche Diagnostics, Indianapolis, Ind.). About 10 days post-fusion, specific antibody-producing hybridoma pools are identified using standard assays (e.g., ELISA). Positive pools may be analyzed further for their ability to block or reduce the activity of the target protein. Positive pools are cloned by limiting dilution.

In certain aspects, the invention also includes the use of multiple monoclonal antibodies that are specific for different epitopes on a single target molecule. Use of such multiple antibodies in combination can reduce carrier effects seen with single antibodies and may also increase rates of clearance via the Fc receptor and improve ADCC. Two, three, or more monoclonal antibodies can be used in combination.

The amino acid sequence of a native antibody can be varied through the application of recombinant DNA techniques. Thus, antibodies can be redesigned to obtain desired characteristics. Modified antibodies can provide, for example, improved stability and/or therapeutic efficacy relative to its non-modified form. The possible variations are many and range from the changing of just one or a few amino acids to the complete redesign of, for example, the variable or constant region. Changes in the constant region will, in general, be made in order to improve or alter characteristics, such as complement fixation, interaction with membranes, and other effector functions. Typically, changes in the variable region will be made in order to improve the antigen binding characteristics, improve variable region stability, or reduce the risk of immunogenicity. Phage display techniques can also be employed. See, e.g., Huse et al., Science 246:1275-1281, 1989; Ladner et al., U.S. Pat. No. 5,571,698.

For therapeutic antibodies for use in humans, it is usually desirable to humanize non-human regions of an antibody according to known procedures. Methods of making humanized antibodies are disclosed, for example, in U.S. Pat. Nos. 5,530,101; 5,821,337; 5,585,089; 5,693,762; and 6,180,370. Typically, a humanized anti-VEGF-A antibody comprises the complementarity determining regions (CDRs) of a mouse donor immunoglobulin and heavy chain and light chain frameworks of a human acceptor immunoglobulin. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323, 1988).

Non-humanized chimeric antibodies can also be used therapeutically (e.g., in immunosuppressed patients). Accordingly, in some variations, an antibody in accordance with the present invention is a chimeric antibody derived, inter alia, from a non-human anti-VEGF-A antibody. Preferably, a chimeric antibody comprises a variable region derived from a mouse or rat antibody and a constant region derived from a human so that the chimeric antibody has a longer half-life and is less immunogenic when administered to a human subject. Methods for producing chimeric antibodies are known in the art. (See e.g., Morrison, Science 229:1202, 1985; Oi et al., BioTechniques 4:214, 1986; Gillies et al., J. Immunol. Methods 125:191-202, 1989; U.S. Pat. Nos. 5,807,715; 4,816,567; and 4,816,397.)

The present invention also encompasses fully human antibodies such as those derived from peripheral blood mononuclear cells of ovarian, breast, renal, colorectal, lung, endometrial, or brain cancer patients. Such cells may be fused with myeloma cells, for example, to form hybridoma cells producing fully human antibodies against VEGF-A. Human antibodies can also be made in transgenic, non-human animals, commonly mice. See, e.g., Tomizuka et al., U.S. Pat. No. 7,041,870. In general, a nonhuman mammal is made transgenic for a human heavy chain locus and a human light chain locus, and the corresponding endogenous immunoglobulin loci are inactivated.

Antibodies of the present invention may be specified in terms of an epitope or portion of a VEGF-A polypeptide that they recognize or specifically bind. An epitope or polypeptide portion may be specified, e.g., by N-terminal and C-terminal positions of the epitope or other portion of the VEGF-A polypeptide shown in SEQ ID NO:72.

The antibodies of the invention have binding affinities that include a dissociation constant (K_{d}) less than 5×10⁻² M, less than 10⁻² M, less than 5×10⁻³ M, less than 10⁻³ M, less than 5×10⁻⁴ M, less than 10⁻⁴ M, less than 5×10⁻⁵ M, less than 10⁻⁵ M, less than 5×10⁻⁶ M, less than 10⁻⁶ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M.

Antibodies of the present invention further include derivatives that are modified, e.g., by the covalent attachment of any type of molecule to the antibody such that covalent attachment does not prevent the antibody from binding to its epitope. Suitable modifications include, for example, fucosylation, glycosylation, acetylation, pegylation, phosphorylation, and amidation. The antibodies and derivatives thereof may themselves by derivatized by known protecting/blocking groups, proteolytic cleavage, linkage to a cellular ligand or other proteins, and the like. In some embodiments of the invention, at least one heavy chain of the antibody is fucosylated. In particular variations, the fucosylation is N-linked. In some certain preferred embodiments, at least one heavy chain of the antibody comprises a fucosylated, N-linked oligosaccharide.

Antibodies of the present invention may be used alone or as immunoconjugates with a cytotoxic agent. In some embodiments, the agent is a chemotherapeutic agent. In other embodiments, the agent is a radioisotope such as, for example, Lead-212, Bismuth-212, Astatine-211, Iodine-131, Scandium-47, Rhenium-186, Rhenium-188, Yttrium-90, Iodine-123, Iodine-125, Bromine-77, Indium-111, or a fissionable nuclide such as Boron-10 or an Actinide. In yet other embodiments, the agent is a toxin or cytotoxic drug such as, for example, ricin, modified Pseudomonas enterotoxin A, calicheamicin, adriamycin, 5-fluorouracil, an auristatin (e.g., auristatin E), maytansin, or the like. Methods of conjugation of antibodies and antibody fragments to such agents are known in the art.

Antibodies of the present invention include variants having single or multiple amino acid substitutions, deletions, additions, or replacements relative to a reference antibody (e.g., a reference antibody having VL and/or VH sequences as shown in Table 2 or Table 3), such that the variant retains one or more biological properties of the reference antibody (e.g., block the binding VEGF-A to their respective counter-structures (a VEGF-A receptor), block the biological activity of VEGF-A, binding affinity). The skilled person can produce variants having single or multiple amino acid substitutions, deletions, additions, or replacements. These variants may include, for example: (a) variants in which one or more amino acid residues are substituted with conservative or nonconservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety, and the like. Antibodies of the invention may include variants in which amino acid residues from one species are substituted for the corresponding residue in another species, either at the conserved or nonconserved positions. In another embodiment, amino acid residues at nonconserved positions are substituted with conservative or nonconservative residues. The techniques for obtaining these variants, including genetic (suppressions, deletions, mutations, etc.), chemical, and enzymatic techniques, are known to the person having ordinary skill in the art.

Exemplary antibodies that bind to VEGF-A have been identified by screening a phage display library. Methods of screening by phage display are described in detail in standard reference texts, such as Babas, Phage Display: A Laboratory Manual (Cold Spring Harbor Lab Press, 2001) and Lo, Benny K. C., A., Antibody Engineering (2004). Such phage display libraries can be used to display expressed proteins on the surface of a cell or other substance such that the complementary binding entity can be functionally isolated. In one such phage display library, the antibody light-chain variable region and a portion of the heavy-chain variable region are combined with synthetic DNA encoding human antibody sequences, which are then displayed on phage and phagemid libraries as Fab antibody fragments (Dyax® Human Antibody Libraries, Dyax Corp., Cambridge, Mass.). Thus, the variable light and heavy chain fragments of antibodies can be isolated in a Fab format. These variable regions can then be manipulated to generate antibodies, including antigen-binding fragments, such as scFvs, bispecific scFvs, and multispecific, multifunctional antagonists to VEGF-A.

Using this technology, the variable regions of exemplary Fabs have been identified for their characteristics of binding and/or neutralizing VEGF-A in assays described herein. (See Examples, infra.) These variable regions were manipulated to generate various binding entities, including scFvs that bind and/or neutralize VEGF-A. Table 2 below show nucleotide and amino acid SEQ ID NO. designations for anti-VEGF-A antibody clusters identified for their ability to bind and neutralize VEGF-A, while Table 3 list the amino acid residue positions corresponding to the framework and CDR regions of the anti-VEGF-A antibodies listed in Table 2.

TABLE 4
SEQ ID NO. Designations for anti-VEGF-A Antibody Clusters

| | | V_{L} | | V_{H} |
|---|---|---|---|---|
| | V_{L} nucleotide | polypeptide | V_{H} nucleotide | polypeptide |
| Cluster # | SEQ ID NO: | SEQ ID NO: | SEQ ID NO: | SEQ ID NO: |
| 870 | 47 | 48 | 49 | 50 |
| 1039 | 65 | 66 | 67 | 68 |
| 1094 | 53 | 54 | 55 | 56 |

TABLE 5
Amino Acid Residue Positions** Corresponding to Framework and CDR Regions of anti-VEGF-A Antibodies

| | Light | Light | Light | Light | Light | Light | | Heavy | Heavy | Heavy | Heavy | Heavy | Heavy | Heavy |
|---|---|---|---|---|---|---|---|---|---|---|---|---|---|---|
| Cluster | FR1 | CDR1 | FR2 | CDR2 | FR3 | CDR3 | Light FR4 | FR1 | CDR1 | FR2 | CDR2 | FR3 | CDR3 | FR4 |
| # | range | range | range | range | range | range | range | range | range | range | range | range | range | range |
| 870 | 1-22 | 23-35 | 36-50 | 51-57 | 58-89 | 90-100 | 101-110 | 1-30 | 31-35 | 36-49 | 50-66 | 67-98 | 99-102 | 103-113 |
| 1039 | 1-23 | 24-34 | 35-49 | 50-56 | 57-88 | 89-97 | 98-107 | 1-30 | 31-35 | 36-49 | 50-66 | 67-98 | 99-110 | 111-121 |
| 1094 | 1-22 | 23-35 | 36-50 | 51-57 | 58-89 | 90-100 | 101-110 | 1-30 | 31-35 | 36-49 | 50-66 | 67-98 | 99-102 | 103-113 |

**Residue position numbers shown (indicated as a “range” of residue positions of either the variable light (VL) or variable heavy (VH) chain polypeptide) are according to VL or VH polypeptide sequences for the corresponding antibody cluster number, the amino acid SEQ ID NOs: for which are indicated in Table 4.

In some embodiments, an anti-VEGF-A antibody of the present invention comprises one or more CDRs of an anti-VEGF-A antibody listed in Table 2 (boundaries of corresponding CDR regions shown in Table 3, respectively). For example, in certain variations, the antibody comprises a heavy chain CDR (at least one of the HCDR1, HCDR2, and HCDR3 regions) and/or a corresponding light chain CDR (at least one of the LCDR1, LCDR2, and LCDR3 regions) of an antibody listed in Table 2. In typical embodiments, the anti-VEGF-A antibody has two or three heavy chain CDRs and/or two or three light chain CDRs of an antibody listed in Table 2. In some variations, where an anti-VEGF-A antibody has at least one heavy chain CDR an antibody listed in Table 2, the antibody further comprises at least one corresponding light chain CDR.

In particular variations, an anti-VEGF-A antibody includes a heavy and/or light chain variable domain, the heavy or light chain variable domain having (a) a set of three CDRs corresponding to the heavy or light chain CDRs as shown for an antibody listed in Table 2, and (b) a set of four framework regions. For example, an anti-VEGF-A antibody can include a heavy and/or light chain variable domain, where the heavy or light chain variable domain has (a) a set of three CDRs, in which the set of CDRs are from an antibody listed in Table 2, and (b) a set of four framework regions, in which the set of framework regions are identical to or different from the set of framework regions of the same antibody listed in Table 2.

In specific embodiments, an anti-VEGF-A antibody includes a heavy chain variable region and/or light chain variable region that is substantially identical to the heavy and/or light chain variable region(s) of an antibody listed in Table 2.

In some embodiments of an anti-VEGF-A antibody in accordance with the present invention, LCDR1 has the amino acid sequence shown in residues 24-34 of SEQ ID NO:66; LCDR2 has the amino acid sequence shown in residues 50-56 of SEQ ID NO:66; LCDR3 has the amino acid sequence shown in residues 89-97 of SEQ ID NO:66; HCDR1 has the HCDR1 amino acid sequence of antibody c1039 (residues 31-35 of SEQ ID NO:68); HCDR2 has the HCDR2 amino acid sequence of antibody c1039 (residues 50-66 of SEQ ID NO:68); and HCDR3 has an amino acid sequence selected from the group consisting of SEQ ID NOs:68.

In other embodiments of an anti-VEGF-A antibody in accordance with the present invention, LCDR1 has the LCDR1 amino acid sequence of an antibody selected from the group consisting of c870 and c1094 (residues 23-35 of SEQ ID NOS:48 and 54, respectively); LCDR2 has the LCDR2 amino acid sequence of an antibody selected from the group consisting of c870 and c1094 (residues 51-57 of SEQ ID NOS:48 and 54, respectively); LCDR3 has the LCDR3 amino acid sequence of an antibody selected from the group consisting of c870 and c1094 (residues 90-100 of SEQ ID NOS:48 and 54, respectively); HCDR1 has the HCDR1 amino acid sequence of an antibody selected from the group consisting of c870 and c1094 (residues 31-35 of SEQ ID NOS:48 and 54, respectively); HCDR2 has the HCDR2 amino acid sequence of an antibody selected from the group consisting of c870 and c1094 (residues 50-66 of SEQ ID NOS:48 and 54, respectively); and HCDR3 has an amino acid sequence selected from the group consisting of residues 99-102 of SEQ ID NOS:48 and 54, respectively. In specific variations, the anti-VEGF-A antibody has CDRs LCDR1, LCDR2, LCDR3, HCDR1, HCDR2, and HCDR3 of an antibody selected from the group consisting of c870, c1039 and c1094. For example, in particular embodiments, the anti-VEGF-A antibody has the light and heavy chain variable domains (V_{L} and V_{H}) of an antibody selected from the group consisting of c870, c1039 and c1094.

In other embodiments, an anti-VEGF-A antibody in accordance with the present invention comprises a V_{L} domain comprising CDRs LCDR1, LCDR2, and LCDR3 and a V_{H} domain comprising CDRs HCDR1, HCDR2, and HCDR3, wherein said set of V_{L} and V_{H} CDRs has 3 or fewer amino acid substitutions relative to a second set of CDRs, where said second set of CDRs has the LCDR1, LCDR2, LCDR3, HCDR1, HCDR2, and HCDR3 amino acid sequences of an antibody selected from group consisting of c870, c1039 and c1094. In particular variations, the antibody comprises zero, one, or two amino acid substitutions in said set of CDRs.

Epitopes recognized by anti-VEGF-A antibodies of the present invention typically include five or more amino acids of human VEGF-A₁₆₅ (residues 27-191 of SEQ ID NO:72). Preferred epitopes comprise at least one amino acid included within one or more of the following polypeptide regions of VEGF-A: HEVVKFMDVYQRSYCHPIETL (amino acid residues 38-58 of SEQ ID NO:72), EYIFKPSCVPLMRCG (amino acid residues 70-84 of SEQ ID NO:72), EESNITMQIMRIKPHQG (amino acid residues 98-114 of SEQ ID NO:72), and PCGPCSERRKHLF (amino acid residues 142-154). In certain embodiments, the epitope comprises at least two, at least three, at least four, at least five, at least six, or at least seven amino acids from one or more of the VEGF-A polypeptide regions as shown in residues 38-58, 70-84, 98-114, and 142-154 of SEQ ID NO:72. In some variations, such VEGF-A epitopes are epitopes as determined by peptide microarray epitope mapping comprising the use of overlapping VEGF-A peptides (e.g., 13-mer peptides, with, for example, 2 amino acid shifts between each pair of sequential peptides).

In particular variations of an anti-VEGF-A antibody as above, the anti-VEGF-A epitope comprises at least one amino acid included within one or more of the following polypeptide regions of VEGF-A: KFMDVYQRSYC (amino acid residues 42-52 of SEQ ID NO:72), IFKPSCVPLMR (amino acid residues 72-82 of SEQ ID NO:72), IMRIKPHQG (amino acid residues 106-114 of SEQ ID NO:72), and PCGPCSERRKHLF (amino acid residues 142-154). In certain embodiments, the epitope comprises at least two, at least three, at least four, at least five, at least six, or at least seven amino acids from one or more of the VEGF-A polypeptide regions as shown in residues 42-52, 72-82, 106-114, and 142-154 of SEQ ID NO:72.

In some related variations, an anti-VEGF-A antibody in accordance with the present invention binds to an epitope comprising (a) one or more amino acids included within a first polypeptide region of VEGF-A as shown in amino acid residues 38-58 or 42-52 of SEQ ID NO:72 and (b) one or more amino acids included within a second polypeptide region of VEGF-A as shown in amino acid residues 70-84 or 72-82 of SEQ ID NO:72.

In certain embodiments of an anti-VEGF-A antibody binding to an epitope comprising (a) and (b) as above, the epitope does not comprise an amino acid included within a polypeptide region of VEGF-A as shown in residues 90 to 132 of SEQ ID NO:72 (EGLECVPTEESNITMQIMRIKPHQGQHIGEMSFLQHNKCECRP).

In other embodiments of an anti-VEGF-A antibody binding to an epitope comprising (a) and (b) as above, the epitope further comprises (c) one or more amino acids included within a third polypeptide region of VEGF-A as shown in residues 96-114 or 106-114 of SEQ ID NO:72.

In some embodiments of an anti-VEGF-A antibody binding to an epitope comprising (a), (b), and (c) as above, the antibody does not bind to human and mouse VEGF-A with K_{d} values within 10-fold of the other.

In yet other variations of an anti-VEGF-A antibody binding to an epitope comprising (a) and (b) as above, the epitope further comprises (d) one or more amino acids included within a fourth polypeptide region of VEGF-A as shown in residues 142-154 of SEQ ID NO:72.

In certain embodiments, an anti-VEGF-A antibody is an antibody fragment such as, for example, an Fv, Fab, Fab′, F(ab)₂, F(ab′)₂, scFv, or diabody. In some preferred embodiments, an anti-VEGF-A antibody is an scFv. scFv entities that bind VEGF-A can be oriented with the variable light (V_{L}) region either amino terminal to the variable heavy (V_{H}) region or carboxylterminal to it. In some variations, an anti-VEGF-A scFv has the CDRs of an anti-VEGF-A antibody listed in Table X. In particular variations, an anti-VEGF-A scFv has the V_{L} and V_{H} domains of an anti-VEGF-A antibody listed in Table 2. In certain embodiments, the CDRs or the V_{L} and V_{H} domains of an anti-VEGF-A scFv are those of an anti-VEGF-A antibody selected from c870 c1039 and c1094. In specific variations of an anti-VEGF-A scFv, the scFv comprises an amino acid sequence as set forth in SEQ ID NO:70 (c1039 scFv; nucleotide sequence shown in SEQ ID NO:69); SEQ ID NO:44 (c870.1e6 scFv; nucleotide sequence shown in SEQ ID NO:43); or SEQ ID NO:46 (c1094.1 scFv; nucleotide sequence shown in SEQ ID NO:45). Additionally, scFvs may be provided in any of a variety of bispecific antibody formats such as, for example, tandem scFv (tascFv), bi-single chain Fv (biscFv), and whole monoclonal antibody with a single chain Fv (scFv) fused to the carboxyl terminus (biAb) (see infra).

C. Bispecific Antibody/Soluble Receptor Combinations Conjugated Using Fc Proteins

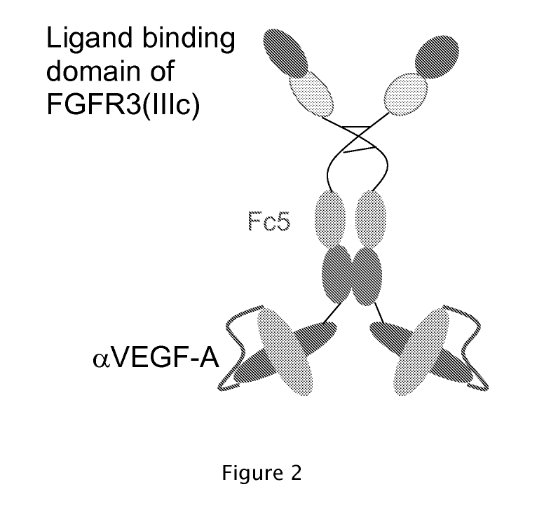

Bispecific binding proteins combine the binding proteins of this invention via the Fc region of an immunoglobulin heavy chain as exemplified in FIG. 2. The Fc-fusion protein comprises the Fc region of an IgG molecule. In a further embodiment, the Fc region is from a human IgG1 molecule. In some embodiments, the immunoglobulin fusion includes the hinge, CH2 and CH3, or the hinge, CH1, CH2 and CH3 regions of an IgG1 molecule.

For the production of immunoglobulin fusions, see also U.S. Pat. No. 5,428,130, U.S. Pat. No. 5,843,725, U.S. Pat. No. 6,018,026, and Chamow et al., TIBTECH, 14: 52-60 (1996).

The simplest and most straightforward Fc-fusion protein design often combines the binding domain(s) of antagonist polypeptides of this invention, via the Fc region of an immunoglobulin heavy chain. In the he Fc-fusion proteins of the present invention, nucleic acid encoding the binding components will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion, or binding characteristics of the Fc-fusion protein.

In a preferred embodiment, the binding domain sequence is fused to the N-terminus of the Fc region of immunoglobulin G1(IgG1). It is possible to fuse the entire heavy chain constant region to the binding domain sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the binding domain amino acid sequence is fused to (a) the hinge region and CH2 and CH3 or (b) the CH1, hinge, CH2 and CH3 domains, of an IgG heavy chain.

For bispecific Fc-fusion proteins, the Fc-fusion proteins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Alternatively, the Fc sequences can be inserted between immunoglobulin heavy chain and light chain sequences, such that an immunoglobulin comprising a chimeric heavy chain is obtained. In this embodiment, the Fc sequences are fused to the 3′ end of an immunoglobulin heavy chain in each arm of an immunoglobulin, either between the hinge and the CH2 domain, or between the CH2 and CH3 domains. Similar constructs have been reported by Hoogenboom et al., Mol. Immunol., 28: 1027-1037 (1991).

Although the presence of an immunoglobulin light chain is not required in the Fc-fusion proteins of the present invention, an immunoglobulin light chain might be present either covalently associated to an binding domain-immunoglobulin heavy chain fusion polypeptide, or directly fused to the binding domain. In the former case, DNA encoding an immunoglobulin light chain is typically coexpressed with the DNA encoding the binding domain-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567.

Fc-fusion proteins are most conveniently constructed by fusing the cDNA sequence encoding the binding domain portion in-frame to an immunoglobulin cDNA sequence. However, fusion to genomic immunoglobulin fragments can also be used (see, e.g. Aruffo et al., Cell, 61: 1303-1313 (1990); and Stamenkovic et al., Cell, 66: 1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the binding domain and the immunoglobulin parts of the Fc-fusion protein are inserted in tandem into a plasmid vector that directs efficient expression in the chosen host cells.

Particular modifications have been made to produce Fc sequences useful for creating Fc fusion molecules for use in the present invention. Specifically, six versions of a modified human IgG1 Fc were generated for creating Fc fusion proteins and are named Fc-488 (SEQ ID NO:76), as well as Fc4 (SEQ ID NO:77), Fc5 (SEQ ID NO:74), Fc6 (SEQ ID NO:78), and Fc7 (SEQ ID NO:79). Fc4, Fc5, and Fc6 contain mutations to reduce effector functions mediated by the Fc by reducing FcγRI binding and complement C1q binding Fc4 contains the same amino acid substitutions that were introduced into Fc-488. Additional amino acid substitutions were introduced to reduce potential Fc mediated effector functions. Specifically, three amino acid substitutions were introduced to reduce FcγRI binding. These are the substitutions at EU index positions 234, 235, and 237. Substitutions at these positions have been shown to reduce binding to FcγRI (Duncan et al., Nature 332:563 (1988)). These amino acid substitutions may also reduce FcγRIIa binding, as well as FcγRIII binding (Sondermann et al., Nature 406:267 (2000); Wines et al., J. Immunol. 164:5313 (2000)).

Several groups have described the relevance of EU index positions 330 and 331 in complement C1q binding and subsequent complement fixation (Canfield and Morrison, J. Exp. Med. 173:1483 (1991); Tao et al., J. Exp. Med. 178:661 (1993)). Amino acid substitutions at these positions were introduced in Fc4 to reduce complement fixation. The C_{H3} domain of Fc4 is identical to that found in the corresponding wild-type polypeptide, except for the stop codon, which was changed from TGA to TAA to eliminate a potential dam methylation site when the cloned DNA is grown in dam plus strains of E. coli.

In Fc5, the arginine residue at EU index position 218 was mutated back to a lysine, because the BglII cloning scheme was not used in fusion proteins containing this particular Fc. The remainder of the Fc5 sequence matches the above description for Fc4.

Fc6 is identical to Fc5 except that the carboxyl terminal lysine codon has been eliminated. The C-terminal lysine of mature immunoglobulins is often removed from mature immunoglobulins post-translationally prior to secretion from B-cells, or removed during serum circulation. Consequently, the C-terminal lysine residue is typically not found on circulating antibodies. As in Fc4 and Fc5 above, the stop codon in the Fc6 sequence was changed to TAA.

Fc7 is identical to the wild-type γ1 Fc except for an amino acid substitution at EU index position 297 located in the C_{H2} domain. EU index position Asn-297 is a site of N-linked carbohydrate attachment. N-linked carbohydrate introduces a potential source of variability in a recombinantly expressed protein due to potential batch-to-batch variations in the carbohydrate structure. In an attempt to eliminate this potential variability, Asn-297 was mutated to a glutamine residue to prevent the attachment of N-linked carbohydrate at that residue position. The carbohydrate at residue 297 is also involved in Fc binding to the FcRIII (Sondermann et al., Nature 406:267 (2000)). Therefore, removal of the carbohydrate should decrease binding of recombinant Fc7 containing fusion proteins to the FcγRs in general. As above, the stop codon in the Fc7 sequence was mutated to TAA.

Leucine zipper forms of these molecules are also contemplated by the invention. “Leucine zipper” is a term in the art used to refer to a leucine rich sequence that enhances, promotes, or drives dimerization ortrimerization of its fusion partner (e.g., the sequence or molecule to which the leucine zipper is fused or linked to). Various leucine zipper polypeptides have been described in the art. See, e.g., Landschulz et al., Science, 240: 1759 (1988); U.S. Pat. No. 5,716,805; WO 94/10308; et al., FEBS Letters, 344: 1991 (1994); Maniatis et al., Nature, 341: 24 (1989). Those skilled in the art will appreciate that a leucine zipper sequence may be fused at either the 5′ or 3′ end of the polypeptide of this invention.

Fusion proteins may generally be prepared using standard techniques, including chemical conjugation. Fusion proteins can also be expressed as recombinant proteins in an expression system by standard techniques. Suitable linkers are further described herein, infra.

A linker can be naturally-occurring, synthetic, or a combination of both. For example, a synthetic linker can be a randomized linker, e.g., both in sequence and size. In one aspect, the randomized linker can comprise a fully randomized sequence, or optionally, the randomized linker can be based on natural linker sequences. The linker can comprise, for example, a non-polypeptide moiety (e.g., a polynucleotide), a polypeptide, or the like.

A linker can be rigid, or alternatively, flexible, or a combination of both. Linker flexibility can be a function of the composition of both the linker and the subunits that the linker interacts with. The linker joins two selected binding entities (e.g., two separate polypeptides or proteins, such as two different antibodies) and maintains the entities as separate and discrete. The linker can allow the separate, discrete domains to cooperate yet maintain separate properties such as multiple separate binding sites for the same target in a multimer or, for example, multiple separate binding sites for different targets in a multimer. In some cases, a disulfide bridge exists between two linked binding entities or between a linker and a binding entity.

Choosing a suitable linker for a specific case where two or more binding entities are to be connected may depend on a variety of parameters including, e.g., the nature of the binding entities, the structure and nature of the target to which the bispecific composition should bind, and/or the stability of the linker (e.g., peptide linker) towards proteolysis and oxidation.

Particularly suitable linker polypeptides predominantly include amino acid residues selected from Glycine (Gly), Serine (Ser), Alanine (Ala), and Threonine (Thr). For example, the peptide linker may contain at least 75% (calculated on the basis of the total number of residues present in the peptide linker), such as at least 80%, at least 85%, or at least 90% of amino acid residues selected from Gly, Ser, Ala, and Thr. The peptide linker may also consist of Gly, Ser, Ala and/or Thr residues only. The linker polypeptide should have a length that is adequate to link two binding entities in such a way that they assume the correct conformation relative to one another so that they retain the desired activity, such as binding to a target molecule as well as other activities that may be associated with such target binding (e.g., agonistic or antagonistic activity for a given biomolecule).

A suitable length for this purpose is, e.g., a length of at least one and typically fewer than about 50 amino acid residues, such as 2-25 amino acid residues, 5-20 amino acid residues, 5-15 amino acid residues, 8-12 amino acid residues or 11 residues. Other suitable polypeptide linker sizes may include, e.g., from about 2 to about 15 amino acids, from about 3 to about 15, from about 4 to about 12, about 10, about 8, or about 6 amino acids. The amino acid residues selected for inclusion in the linker polypeptide should exhibit properties that do not interfere significantly with the activity or function of the polypeptide multimer. Thus, the peptide linker should, on the whole, not exhibit a charge that would be inconsistent with the activity or function of the multimer, or interfere with internal folding, or form bonds or other interactions with amino acid residues in one or more of the domains that would seriously impede the binding of the multimer to the target in question.

The use of naturally occurring as well as artificial peptide linkers to connect polypeptides into novel linked fusion polypeptides is well-known in the art. (See, e.g., Hallewell et al., J. Biol. Chem. 264, 5260-5268, 1989; Alfthan et al., Protein Eng. 8, 725-731, 1995; Robinson and Sauer, Biochemistry 35, 109-116, 1996; Khandekar et al., J. Biol. Chem. 272, 32190-32197, 1997; Fares et al., Endocrinology 139, 2459-2464, 1998; Smallshaw et al., Protein Eng. 12, 623-630, 1999; U.S. Pat. No. 5,856,456.)

One example where the use of peptide linkers is widespread is for production of single-chain antibodies where the variable regions of a light chain (V_{L}) and a heavy chain (V_{H}) are joined through an artificial linker, and a large number of publications exist within this particular field. A widely used peptide linker is a 15 mer consisting of three repeats of a Gly-Gly-Gly-Gly-Ser amino acid sequence ((Gly₄Ser)₃) (SEQ ID NO:73). Other linkers have been used, and phage display technology, as well as selective infective phage technology, has been used to diversify and select appropriate linker sequences (Tang et al., J. Biol. Chem. 271, 15682-15686, 1996; Hennecke et al., Protein Eng. 11, 405-410, 1998). Peptide linkers have been used to connect individual chains in hetero- and homo-dimeric proteins such as the T-cell receptor, the lambda Cro repressor, the P22 phage Arc repressor, IL-12, TSH, FSH, IL-5, and interferon-γ. Peptide linkers have also been used to create fusion polypeptides. Various linkers have been used, and, in the case of the Arc repressor, phage display has been used to optimize the linker length and composition for increased stability of the single-chain protein (see Robinson and Sauer, Proc. Natl. Acad. Sci. USA 95, 5929-5934, 1998).

Still another way of obtaining a suitable linker is by optimizing a simple linker (e.g., (Gly₄Ser)ₙ) through random mutagenesis.

As discussed above, it is generally preferred that the peptide linker possess at least some flexibility. Accordingly, in some variations, the peptide linker contains 1-25 glycine residues, 5-20 glycine residues, 5-15 glycine residues, or 8-12 glycine residues. Particularly suitable peptide linkers typically contain at least 50% glycine residues, such as at least 75% glycine residues. In some embodiments, a peptide linker comprises glycine residues only. In certain variations, the peptide linker comprises other residues in addition to the glycine. Preferred residues in addition to glycine include Ser, Ala, and Thr, particularly Ser.

In some cases, it may be desirable or necessary to provide some rigidity into the peptide linker. This may be accomplished by including proline residues in the amino acid sequence of the peptide linker. Thus, in another embodiment, a peptide linker comprises at least one proline residue in the amino acid sequence of the peptide linker. For example, a peptide linker can have an amino acid sequence wherein at least 25% (e.g., at least 50% or at least 75%) of the amino acid residues are proline residues. In one particular embodiment of the invention, the peptide linker comprises proline residues only.

In some embodiments, a peptide linker is modified in such a way that an amino acid residue comprising an attachment group for a non-polypeptide moiety is introduced. Examples of such amino acid residues may be a cysteine or a lysine residue (to which the non-polypeptide moiety is then subsequently attached). Another alternative is to include an amino acid sequence having an in vivo N-glycosylation site (thereby attaching a sugar moiety (in vivo) to the peptide linker). An additional option is to genetically incorporate non-natural amino acids using evolved tRNAs and tRNA synthetases (see, e.g., U.S. Patent Application Publication 2003/0082575) into a polypeptide binding entity or peptide linker. For example, insertion of keto-tyrosine allows for site-specific coupling to an expressed polypeptide.

In certain variations, a peptide linker comprises at least one cysteine residue, such as one cysteine residue. For example, in some embodiments, a peptide linker comprises at least one cysteine residue and amino acid residues selected from the group consisting of Gly, Ser, Ala, and Thr. In some such embodiments, a peptide linker comprises glycine residues and cysteine residues, such as glycine residues and cysteine residues only. Typically, only one cysteine residue will be included per peptide linker. One example of a specific peptide linker comprising a cysteine residue includes a peptide linker having the amino acid sequence Glyₙ-Cys-Glyₘ, wherein n and m are each integers from 1-12, e.g., from 3-9, from 4-8, or from 4-7.

As previously noted, the present invention comprise bispecific binding proteins comprising a bispecific antibody/soluble receptor combination of an FGFR and an anti-VEGF-A antibody. In some such embodiments, the FGFR and anti-VEGF-A antibodies are covalently linked (e.g., via a peptide linker) to form a bispecific binding protein. In some variations, the bispecific binding protein comprises an immunoglobulin heavy chain constant region such as, for example, an Fc fragment. Particularly suitable Fc fragments include, for example, Fc fragments comprising an Fc region modified to reduce or eliminate one or more effector functions (e.g., Fc5, having the amino acid sequence shown in SEQ ID NO:74).

For example, in some embodiments, a VEGF-A antibody/soluble FGF receptor bispecific binding protein that reduces the activity of both VEGF-A and FGF in accordance with the present invention comprises a binding region of an anti-VEGF-A antibody moiety as described herein and a FGF binding moiety of an FGFR3 as described herein. In certain embodiments the FGF binding moiety is an FGFR3III_{c} as described herein. In certain embodiments the FGF binding moiety is an FGF receptor moiety, and can be FGFR3, and in particular is FGFR3_{IIIc} as described herein. In certain embodiments, a bispecific antibody/soluble receptor protein comprises an FGF receptor moiety that is an FGFR3 selected from the group consisting of FGFR3_{IIIc}(23-375) as shown in SEQ ID NO:13, FGFR3_{IIIc}(23-375)(S249W) as shown in SEQ ID NO:2, FGFR3_{IIIc}(143-375) as shown in SEQ ID NO:19, FGFR3_{IIIc}(143-375)(S249W), as shown in SEQ ID NO:10, FGFR3_{IIIc}(23-375)(P250R) as shown in SEQ ID NO:15, and FGFR3_{IIIc}(143-375)(P250R) as shown in SEQ ID NO:22; in combination with a VEGF-A antibody moiety selected from the group consisting of c870.1e6 scFV as shown in SEQ ID NO:44, c1094.1 scFV as shown in SEQ ID NO:46, c870 scFV as shown in SEQ ID NO:52, and c1039 scFV as shown in SEQ ID NO:70. In other embodiments, a bispecific antibody/soluble receptor combination comprises an FGF binding moiety that is an FGFR3 selected from the group consisting of FGFR3_{IIIc}(23-375) as shown in SEQ ID NO:13, FGFR3_{IIIc}(23-375)(S249W) as shown in SEQ ID NO:2, FGFR3_{IIIc}(143-375) as shown in SEQ ID NO:19, FGFR3_{IIIc}(143-375)(S249W), as shown in SEQ ID NO:10, FGFR3_{IIIc}(23-375)(P250R) as shown in SEQ ID NO:15, FGFR3_{IIIc}(143-375)(P250R) as shown in SEQ ID NO:22, and VEGF-A binding moiety selected from the group consisting of a c870 VL as shown in SEQ ID NO:48 and VH as shown in SEQ ID NO:50, a c1094 VL as shown in SEQ ID NO:54 and VH as shown in SEQ ID NO:56, and a 1039 VL as shown in SEQ ID NO:66 and VH as shown in SEQ ID NO:68.

In other embodiments, the bispecific binding protein of the present invention embodies an FGFR3 moiety and VEGF-A antibody moiety selected from the group consisting of FGFR3(143-375)(S249W)Fc5 c1094.1 pZMP31 (SEQ ID NO:58); FGFR3(23-375)(S249W)Fc5 c1094.1 pZMP31 (SEQ ID NO:60); FGFR3(143-375)(S249W)Fc5 c870e6 pZMP31 (SEQ ID NO:62); and FGFR3(23-375)(S249W)Fc5 c870e6 pZMP31 (SEQ ID NO:64).

In other embodiments the FGF binding moiety is FGFR2. In certain embodiments the FGFR2 comprises FGFR2_{IIIc}. In certain embodiments, a bispecific antibody/soluble receptor combinations comprises an FGF binding moiety that is an FGFR2 selected from the group consisting of FGFR2_{IIIc}(22-377) as shown in SEQ ID NO:24, FGFR2_{IIIc}(22-377)(S252W) as shown in SEQ ID NO:29, FGFR2_{IIIc}(22-377)(P253R) as shown in SEQ ID NO:33, FGFR2_{IIIc}(145-377), as shown in SEQ ID NO:37, FGFR2_{IIIc}(145-377)(S252W) as shown in SEQ ID NO:40, and FGFR2_{IIIc}(145-377)(P253R) as shown in SEQ ID NO:42; and VEGF-A binding moiety selected from the group consisting of c870.1e6 scFV as shown in SEQ ID NO:44, c1094.1 scFV as shown in SEQ ID NO:46, c870 scFV as shown in SEQ ID NO:52, and c1039 scFV as shown in SEQ ID NO:70. In other embodiments, a bispecific antibody/soluble receptor combination comprises an FGF binding moiety that is an FGFR2 selected from the group consisting of FGFR_{IIIc}(22-377) as shown in SEQ ID NO:24, FGFR_{IIIc}(22-377)(S252W) as shown in SEQ ID NO:29, FGFR_{IIIc}(22-377)(P253R) as shown in SEQ ID NO:33, FGFR_{IIIc}(145-377), as shown in SEQ ID NO:37, FGFR_{IIIc}(145-377)(S252W) as shown in SEQ ID NO:40, and FGFR_{IIIc}(145-377)(P253R) as shown in SEQ ID NO:42; and VEGF-A binding moiety selected from the group consisting a c870 VL as shown in SEQ ID NO:48 and VH as shown in SEQ ID NO:50, a c1094 VL as shown in SEQ ID NO:54 and VH as shown in SEQ ID NO:56, and a 1039 VL as shown in SEQ ID NO:66 and VH as shown in SEQ ID NO:68.

### III. Nucleic Acids, Host Cells, and Methods for Producing VEGF-A and FGF Bispecific Antibody/Soluble Receptor Combination Proteins

The soluble FGFR polypeptides of the invention can be prepared by expressing a DNA encoding the extracellular domain or portions thereof. For example, a polynucleotide sequence that encodes for a polypeptide which contains residues 36-388 of SEQ ID NO:13 can be used to prepare FGFR3_{IIIc}. An N-terminally truncated FGFR3_{IIIc} can be using a polynucleotide encoding residues 36-268 of SEQ ID NO:19. To prepare FGFR2_{IIIc} a polynucleotide encoding residues 36-391 of SEQ ID NO:24 can be used. In another example, a polynucleotide sequence that encodes for a polypeptide which contains residues 36-268 of SEQ ID NO:37 can be used to prepare an N-terminally truncated FGFR2_{IIIc}. It is preferred that the extracellular domain polypeptides be prepared in a form substantially free of transmembrane and intracellular polypeptide segments. To direct the export of the receptor domain from the host cell, the receptor DNA is linked to a second DNA segment encoding a secretory peptide, such as the receptor's native signal sequence. Other signal sequences that could be used include tPA signal sequence (described in the Examples below), CD33 signal sequence or human growth hormone signal sequence. To facilitate purification of the secreted receptor domain, a C-terminal extension, such as a poly-histidine tag, substance P, Flag™ peptide (Hopp et al., Biotechnology 6:1204-1210, 1988; available from Eastman Kodak Co., New Haven, Conn.) or another polypeptide or protein for which an antibody or other specific binding agent is available, can be fused to the receptor polypeptide.

The invention also includes nucleic acids encoding the heavy chain and/or light chain of the antibodies of the invention. Nucleic acids of the invention include nucleic acids having a region that is substantially identical to a V_{L}- and/or V_{H}-encoding polynucleotide as listed in Table 2, supra. Nucleic acids of the invention also include complementary nucleic acids. In some instances, the sequences will be fully complementary (no mismatches) when aligned. In other instances, there may be up to about a 20% mismatch in the sequences. In some embodiments of the invention are provided nucleic acids encoding both a heavy chain and a light chain of an antibody of the invention. The nucleic acid sequences provided herein can be exploited using codon optimization, degenerate sequence, silent mutations, and other DNA techniques to optimize expression in a particular host, and the present invention encompasses such sequence modifications.

Thus, in some aspects, the present invention provides one or more polynucleotide(s) (e.g., DNA or RNA) that encode an FGFR and/or VEGF-A antibody as described herein. In some variations, a polynucleotide of the present invention encodes a VEGF-A antibody/soluble FGF receptor bispecific binding protein that binds and reduces the activity of both FGF and VEGF-A. Those skilled in the art will readily recognize that, in view of the degeneracy of the genetic code, considerable sequence variation is possible among these polynucleotide molecules.

Nucleic acids of the present invention can be cloned into a vector, such as a plasmid, cosmid, bacmid, phage, artificial chromosome (BAC, YAC) or virus, into which another genetic sequence or element (either DNA or RNA) may be inserted so as to bring about the replication of the attached sequence or element. In some embodiments, the expression vector contains a constitutively active promoter segment (such as but not limited to CMV, SV40, Elongation Factor or LTR sequences) or an inducible promoter sequence such as the steroid inducible pIND vector (Invitrogen), where the expression of the nucleic acid can be regulated. Expression vectors of the invention may further comprise regulatory sequences, for example, an internal ribosomal entry site. The expression vector can be introduced into a cell by, for example, transfection.

Accordingly, proteins for use within the present invention can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook et al., Molecular Cloning: A Laboratory Manual (2nd ed., Cold Spring Harbor Laboratory Press, Cold Spring Harbor, N.Y., 1989), and Ausubel et al., supra.

In general, a DNA sequence encoding a protein of interest is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a recombinant protein into the secretory pathway of a host cell, a secretory signal sequence (also known as a leader sequence, prepro sequence or pre sequence) is provided in the expression vector. The secretory signal sequence may be that of the native form of the recombinant protein, or may be derived from another secreted protein (e.g., t-PA; see U.S. Pat. No. 5,641,655) or synthesized de novo. The secretory signal sequence is operably linked to the protein-encoding DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830). In particular variations, a secretory signal sequence for use in accordance with the present invention has an amino acid sequence selected from the group consisting of residues 1-35 of SEQ ID NOS:2, 10, 13, 15, 19, 22, 24, 29, 33, 37, 40, 42, 58, 60, 62, and 64.

Cultured mammalian cells are suitable hosts for production of recombinant proteins for use within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Examples of suitable mammalian host cells include African green monkey kidney cells (Vero; ATCC CRL 1587), human embryonic kidney cells (293-HEK; ATCC CRL 1573), baby hamster kidney cells (BHK-21, BHK-570; ATCC CRL 8544, ATCC CRL 10314), canine kidney cells (MDCK; ATCC CCL 34), Chinese hamster ovary cells (CHO-K1; ATCC CCL61; CHO DG44; CHO DXB11 (Hyclone, Logan, Utah); see also, e.g., Chasin et al., Som. Cell. Molec. Genet. 12:555, 1986)), rat pituitary cells (GH1; ATCC CCL82), HeLa S3 cells (ATCC CCL2.2), rat hepatoma cells (H-4-II-E; ATCC CRL 1548) SV40-transformed monkey kidney cells (COS-1; ATCC CRL 1650) and murine embryonic cells (NIH-3T3; ATCC CRL 1658). Additional suitable cell lines are known in the art and available from public depositories such as the American Type Culture Collection, Manassas, Va. Strong transcription promoters can be used, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants.” Cells that have been cultured in the presence of the selective agent and are able to pass the gene of interest to their progeny are referred to as “stable transfectants.” Exemplary selectable markers include a gene encoding resistance to the antibiotic neomycin, which allows selection to be carried out in the presence of a neomycin-type drug, such as G-418 or the like; the gpt gene for xanthine-guanine phosphoribosyl transferase, which permits host cell growth in the presence of mycophenolic acid/xanthine; and markers that provide resistance to zeocin, bleomycin, blastocidin, and hygromycin (see, e.g., Gatignol et al., Mol. Gen. Genet. 207:342, 1987; Drocourt et al., Nucl. Acids Res. 18:4009, 1990). Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g., hygromycin resistance, multi-drug resistance, puromycin acetyltransferase) can also be used.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463.

Insect cells can be infected with recombinant baculovirus, commonly derived from Autographa californica nuclear polyhedrosis virus (AcNPV). See King and Possee, The Baculovirus Expression System: A Laboratory Guide (Chapman & Hall, London); O'Reilly et al., Baculovirus Expression Vectors: A Laboratory Manual (Oxford University Press., New York 1994); and Baculovirus Expression Protocols. Methods in Molecular Biology (Richardson ed., Humana Press, Totowa, N.J., 1995). Recombinant baculovirus can also be produced through the use of a transposon-based system described by Luckow et al. (J. Virol. 67:4566-4579, 1993). This system, which utilizes transfer vectors, is commercially available in kit form (BAC-TO-BAC kit; Life Technologies, Gaithersburg, Md.). The transfer vector (e.g., PFASTBAC1; Life Technologies) contains a Tn7 transposon to move the DNA encoding the protein of interest into a baculovirus genome maintained in E. coli as a large plasmid called a “bacmid.” See Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing a protein-encoding DNA sequence is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses the protein or interest is subsequently produced. Recombinant viral stocks are made by methods commonly used in the art.

For protein production, the recombinant virus is used to infect host cells, typically a cell line derived from the fall armyworm, Spodoptera frugiperda (e.g., Sf9 or Sf21 cells) or Trichoplusia ni (e.g., HIGH FIVE cells; Invitrogen, Carlsbad, Calif.). See generally Glick and Pasternak, Molecular Biotechnology, Principles & Applications of Recombinant DNA (ASM Press, Washington, D.C., 1994). See also U.S. Pat. No. 5,300,435. Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally described in available laboratory manuals (see, e.g., King and Possee, supra; O'Reilly et al., supra; Richardson, supra).

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936; and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii, and Candida maltosa are known in the art. See, e.g., Gleeson et al., J. Gen. Microbiol. 132:3459-3465, 1986; Cregg, U.S. Pat. No. 4,882,279; and Raymond et al., Yeast 14:11-23, 1998. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808; 5,736,383; 5,854,039; and 5,888,768.

Prokaryotic host cells, including strains of the bacteria Escherichia coli, Bacillus, and other genera are also useful host cells within the present invention. Techniques for transforming these hosts and expressing foreign DNA sequences cloned therein are well known in the art (see, e.g., Sambrook et al., supra). When expressing a recombinant protein in bacteria such as E. coli, the protein may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured protein can then be refolded and dimerized by diluting the denaturant, such as by dialysis against a solution of urea and a combination of reduced and oxidized glutathione, followed by dialysis against a buffered saline solution. In the alternative, the protein may be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody or heparin-Sepharose column. Secreted proteins can be recovered from the periplasmic space in a soluble and functional form by disrupting the cells (by, for example, sonication or osmotic shock) to release the contents of the periplasmic space and recovering the protein, thereby obviating the need for denaturation and refolding. Antibodies, including single-chain antibodies, can be produced in bacterial host cells according to known methods. See, e.g., Bird et al., Science 242:423-426, 1988; Huston et al., Proc. Natl. Acad. Sci. USA 85:5879-5883, 1988; and Pantoliano et al., Biochem. 30:10117-10125, 1991.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

Bispecific binding proteins comprising VEGF-A antibody/soluble FGF receptor bispecific binding proteins are purified by conventional protein purification methods, typically by a combination of chromatographic techniques. See generally Affinity Chromatography: Principles &Methods (Pharmacia LKB Biotechnology, Uppsala, Sweden, 1988); Scopes, Protein Purification: Principles and Practice (Springer-Verlag, New York 1994). Proteins comprising an immunoglobulin heavy chain polypeptide can be purified by affinity chromatography on immobilized protein A. Additional purification steps, such as gel filtration, can be used to obtain the desired level of purity or to provide for desalting, buffer exchange, and the like.

Antibodies can be purified from cell culture media by known methods, such as affinity chromatography using conventional columns and other equipment. In a typical procedure, conditioned medium is harvested and may be stored at 4° C. for up to five days. To avoid contamination, a bacteriostatic agent (e.g., sodium azide) is generally added. The pH of the medium is lowered (typically to Ph ˜5.5), such as by the addition of glacial acetic acid dropwise. The lower pH provides for optimal capture of IgG via a protein G resin. The protein G column size is determined based on the volume of the conditioned medium. The packed column is neutralized with a suitable buffer, such as 35 mM NaPO₄, 120 mM NaCl pH 7.2. The medium is then passed over the neutralized protein g resin at a flow rate determined by both the volume of the medium and of the column size. The flowthrough is retained for possible additional passes over the column. The resin with the captured antibody is then washed into the neutralizing buffer. The column is eluted into fractions using an acidic elution buffer, such as 0.1M glycine, pH 2.7 or equivalent. Each fraction is neutralized, such as with 2M tris, pH 8.0 at a 1:20 ratio tris:glycine. Protein containing fractions (e.g., based on A₂₈₀) are pooled. The pooled fractions are buffer exchanged into a suitable buffer, such as 35 mM NaPO₄, 120 mM NaCl pH 7.2 using a desalting column. Concentration is determined by A₂₈₀ using an extinction coefficient of 1.44. Endotoxin levels may be determined by LAL assay. Purified protein may be stored frozen, typically at −80° C.

Cells expressing functional VEGF-A antibody/soluble FGF receptor bispecific binding proteins are used within screening assays. A variety of suitable assays are known in the art. These assays are based on the detection of a biological response in a target cell. One such assay is a cell proliferation assay. Cells are cultured in the presence or absence of a test compound, and cell proliferation is detected by, for example, measuring incorporation of tritiated thymidine or by colorimetric assay based on the metabolic breakdown of 3-(4,5-dimethylthiazol-2-yl)-2,5-diphenyl tetrazolium bromide (MTT) (Mosman, J. Immunol. Meth. 65: 55-63, 1983). An alternative assay format uses cells that are further engineered to express a reporter gene. The reporter gene is linked to a promoter element that is responsive to the receptor-linked pathway, and the assay detects activation of transcription of the reporter gene. A preferred promoter element in this regard is a serum response element, or SRE. (See, e.g., Shaw et al., Cell 56:563-572, 1989.) A preferred such reporter gene is a luciferase gene. (See de Wet et al., Mol. Cell. Biol. 7:725, 1987.) Expression of the luciferase gene is detected by luminescence using methods known in the art. (See, e.g., Baumgartner et al., J. Biol. Chem. 269:29094-29101, 1994; Schenborn and Goiffin, Promega Notes 41:11, 1993.) Luciferase activity assay kits are commercially available from, for example, Promega Corp., Madison, Wis. Target cell lines of this type can be used to screen libraries of chemicals, cell-conditioned culture media, fungal broths, soil samples, water samples, and the like. For example, a bank of cell-conditioned media samples can be assayed on a target cell to identify cells that produce ligand. Positive cells are then used to produce a cDNA library in a mammalian expression vector, which is divided into pools, transfected into host cells, and expressed. Media samples from the transfected cells are then assayed, with subsequent division of pools, re-transfection, subculturing, and re-assay of positive cells to isolate a cloned cDNA encoding the ligand.

### IV. Methods of Treatment

A. General

In another aspect, the present invention provides methods of inhibiting angiogenesis, particularly methods for treatment of diseases or disorders associated with angiogenesis. Generally, such methods include administering to a subject a bispecific binding protein comprising a bispecific antibody/soluble receptor combination in an amount effective to inhibit angiogenesis. More particularly, for therapeutic use, the bispecific binding protein is administered to a subject suffering from, or at an elevated risk of developing, a disease or disorder characterized by increased angiogenesis (a “neovascular disorder”). Neovascular disorders amenable to treatment in accordance with the present invention include, for example, cancers characterized by solid tumor growth (e.g., pancreatic cancer, renal cell carcinoma (RCC), colorectal cancer, non-small cell lung cancer (NSCLC), glioblastoma, and gastrointestinal stromal tumor (GIST)) as well as various neovascular ocular disorders (e.g., age-related macular degeneration, diabetic retinopathy, iris neovascularization, and neovascular glaucoma). Other neovascular disorders amenable to treatment in accordance with the present invention include, for example, rheumatoid arthritis, psoriasis, atherosclerosis, chronic inflammation, lung inflammation, preeclampsia, pericardial effusion (such as that associated with pericarditis), and pleural effusion.

In each of the embodiments of the treatment methods described herein, the bispecific binding protein comprising a bispecific binding protein comprising a VEGF-A antibody/soluble FGF receptor bispecific binding protein is delivered in a manner consistent with conventional methodologies associated with management of the disease or disorder for which treatment is sought. In accordance with the disclosure herein, an effective amount of the antagonists is administered to a subject in need of such treatment for a time and under conditions sufficient to prevent or treat the disease or disorder.

Subjects for administration of bispecific binding proteins as described herein include patients at high risk for developing a particular disease or disorder associated with angiogenesis as well as patients presenting with an existing neovascular disorder. In certain embodiments, the subject has been diagnosed as having the disease or disorder for which treatment is sought. Further, subjects can be monitored during the course of treatment for any change in the disease or disorder (e.g., for an increase or decrease in clinical symptoms of the disease or disorder).

In prophylactic applications, pharmaceutical compositions or medicants are administered to a patient susceptible to, or otherwise at risk of, a particular disease in an amount sufficient to eliminate or reduce the risk or delay the outset of the disease. In therapeutic applications, compositions or medicants are administered to a patient suspected of, or already suffering from such a disease in an amount sufficient to cure, or at least partially arrest, the symptoms of the disease and its complications. An amount adequate to accomplish this is referred to as a therapeutically- or pharmaceutically-effective dose or amount. In both prophylactic and therapeutic regimes, agents are usually administered in several dosages until a sufficient response (e.g., inhibition of inappropriate angiogenesis activity) has been achieved. Typically, the response is monitored and repeated dosages are given if the desired response starts to fade.

To identify subject patients for treatment according to the methods of the invention, accepted screening methods may be employed to determine risk factors associated with specific neovascular disorders or to determine the status of an existing disorder identified in a subject. Such methods can include, for example, determining whether an individual has relatives who have been diagnosed with a particular disease. Screening methods can also include, for example, conventional work-ups to determine familial status for a particular disease known to have a heritable component. For example, various cancers are also known to have certain inheritable components. Inheritable components of cancers include, for example, mutations in multiple genes that are transforming (e.g., Ras, Raf, EGFR, cMet, and others), the presence or absence of certain HLA and killer inhibitory receptor (KIR) molecules, or mechanisms by which cancer cells are able to modulate immune suppression of cells like NK cells and T cells, either directly or indirectly (see, e.g., Ljunggren and Malmberg, Nature Rev. Immunol. 7:329-339, 2007; Boyton and Altmann, Clin. Exp. Immunol. 149:1-8, 2007). Toward this end, nucleotide probes can be routinely employed to identify individuals carrying genetic markers associated with a particular disease of interest. In addition, a wide variety of immunological methods are known in the art that are useful to identify markers for specific diseases. For example, various ELISA immunoassay methods are available and well-known in the art that employ monoclonal antibody probes to detect antigens associated with specific tumors. Screening may be implemented as indicated by known patient symptomology, age factors, related risk factors, etc. These methods allow the clinician to routinely select patients in need of the methods described herein for treatment. In accordance with these methods, inhibition of angiogenesis may be implemented as an independent treatment program or as a follow-up, adjunct, or coordinate treatment regimen to other treatments.

For administration, the bispecific binding protein comprising a VEGF-A antibody/soluble FGF receptor bispecific binding protein is formulated as a pharmaceutical composition. A pharmaceutical composition comprising a bispecific VEGF-A antibody/FGFR soluble receptor combination can be formulated according to known methods to prepare pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. (See, e.g., Gennaro (ed.), Remington's Pharmaceutical Sciences (Mack Publishing Company, 19th ed. 1995).) Formulations may further include one or more excipients, preservatives, solubilizers, buffering agents, albumin to prevent protein loss on vial surfaces, etc. Monospecific antagonists can be individually formulated or provided in a combined formulation.

A pharmaceutical composition comprising a bispecific binding protein comprising a VEGF-A antibody/soluble FGF receptor bispecific binding protein is administered to a subject in an effective amount. According to the methods of the present invention, an antagonist may be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For pharmaceutical use for treatment of neovascular ocular disorders, the bispecific binding proteins are typically formulated for intravitreal injection according to conventional methods. For prevention and treatment purposes, an antagonist may be administered to a subject in a single bolus delivery, via continuous delivery (e.g., continuous transdermal delivery) over an extended time period, or in a repeated administration protocol (e.g., on an hourly, daily, or weekly basis).

A “therapeutically effective amount” of a composition is that amount that produces a statistically significant effect, such as a statistically significant reduction in disease progression or a statistically significant improvement in organ function. The exact dose will be determined by the clinician according to accepted standards, taking into account the nature and severity of the condition to be treated, patient traits, etc. Determination of dose is within the level of ordinary skill in the art.

Determination of effective dosages in this context is typically based on animal model studies followed up by human clinical trials and is guided by determining effective dosages and administration protocols that significantly reduce the occurrence or severity of the subject disease or disorder in model subjects. Effective doses of the compositions of the present invention vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, i.e., to optimize safety and efficacy. Accordingly, a therapeutically or prophylactically effective amount is also one in which any undesired collateral effects are outweighed by beneficial effects of inhibiting angiogenesis. For administration of a bispecific binding protein comprising a VEGF-A antibody/soluble FGF receptor bispecific binding protein, a dosage typically ranges from about 0.1 μg to 100 mg/kg or 1 μg/kg to about 50 mg/kg, and more usually 10 μg to 5 mg/kg of the subject's body weight. In more specific embodiments, an effective amount of the agent is between about 1 μg/kg and about 20 mg/kg, between about 10 μg/kg and about 10 mg/kg, or between about 0.1 mg/kg and about 5 mg/kg. Dosages within this range can be achieved by single or multiple administrations, including, e.g., multiple administrations per day or daily, weekly, bi-weekly, or monthly administrations. For example, in certain variations, a regimen consists of an initial administration followed by multiple, subsequent administrations at weekly or bi-weekly intervals. Another regimen consists of an initial administration followed by multiple, subsequent administrations at monthly or bi-monthly intervals. Alternatively, administrations can be on an irregular basis as indicated by monitoring of NK cell activity and/or clinical symptoms of the disease or disorder.

Dosage of the pharmaceutical composition may be varied by the attending clinician to maintain a desired concentration at a target site. For example, if an intravenous mode of delivery is selected, local concentration of the agent in the bloodstream at the target tissue may be between about 1-50 nanomoles of the composition per liter, sometimes between about 1.0 nanomole per liter and 10, 15, or 25 nanomoles per liter depending on the subject's status and projected measured response. Higher or lower concentrations may be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of 10 nanomolar.

A pharmaceutical composition comprising bispecific binding proteins comprising a VEGF-A antibody/soluble FGF receptor bispecific binding protein can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants. (See, e.g., Bremer et al., Pharm. Biotechnol. 10:239, 1997; Ranade, “Implants in Drug Delivery,” in Drug Delivery Systems 95-123 (Ranade and Hollinger, eds., CRC Press 1995); Bremer et al., “Protein Delivery with Infusion Pumps,” in Protein Delivery: Physical Systems 239-254 (Sanders and Hendren, eds., Plenum Press 1997); Yewey et al., “Delivery of Proteins from a Controlled Release Injectable Implant,” in Protein Delivery: Physical Systems 93-117 (Sanders and Hendren, eds., Plenum Press 1997).) Other solid forms include creams, pastes, other topological applications, and the like.

Liposomes provide one means to deliver therapeutic polypeptides to a subject, e.g., intravenously, intraperitoneally, intrathecally, intramuscularly, subcutaneously, or via oral administration, inhalation, or intranasal administration. Liposomes are microscopic vesicles that consist of one or more lipid bilayers surrounding aqueous compartments. (See, generally, Bakker-Woudenberg et al., Eur. J. Clin. Microbiol. Infect. Dis. 12 (Suppl. 1):S61, 1993; Kim, Drugs 46:618, 1993; Ranade, “Site-Specific Drug Delivery Using Liposomes as Carriers,” in Drug Delivery Systems 3-24 (Ranade and Hollinger, eds., CRC Press 1995).) Liposomes are similar in composition to cellular membranes and as a result, liposomes can be administered safely and are biodegradable. Depending on the method of preparation, liposomes may be unilamellar or multilamellar, and liposomes can vary in size with diameters ranging from 0.02 μm to greater than 10 μm. A variety of agents can be encapsulated in liposomes: hydrophobic agents partition in the bilayers and hydrophilic agents partition within the inner aqueous space(s). (See, e.g., Machy et al., Liposomes In Cell Biology And Pharmacology (John Libbey 1987); Ostro et al., American J. Hosp. Pharm. 46:1576, 1989.) Moreover, it is possible to control the therapeutic availability of the encapsulated agent by varying liposome size, the number of bilayers, lipid composition, as well as the charge and surface characteristics of the liposomes.

Liposomes can adsorb to virtually any type of cell and then slowly release the encapsulated agent. Alternatively, an absorbed liposome may be endocytosed by cells that are phagocytic. Endocytosis is followed by intralysosomal degradation of liposomal lipids and release of the encapsulated agents (see Scherphof et al., Ann. N.Y. Acad. Sci. 446:368, 1985). After intravenous administration, small liposomes (0.1 to 1.0 μm) are typically taken up by cells of the reticuloendothelial system, located principally in the liver and spleen, whereas liposomes larger than 3.0 μm are deposited in the lung. This preferential uptake of smaller liposomes by the cells of the reticuloendothelial system has been used to deliver chemotherapeutic agents to macrophages and to tumors of the liver.

The reticuloendothelial system can be circumvented by several methods including saturation with large doses of liposome particles, or selective macrophage inactivation by pharmacological means (see Claassen et al., Biochim. Biophys. Acta 802:428, 1984). In addition, incorporation of glycolipid- or polyethelene glycol-derivatized phospholipids into liposome membranes has been shown to result in a significantly reduced uptake by the reticuloendothelial system (see Allen et al., Biochim. Biophys. Acta 1068:133, 1991; Allen et al., Biochim. Biophys. Acta 1150:9, 1993).

Liposomes can also be prepared to target particular cells or organs by varying phospholipid composition or by inserting receptors or counter-receptors into the liposomes. For example, liposomes, prepared with a high content of a nonionic surfactant, have been used to target the liver. (See, e.g., Japanese Patent 04-244,018 to Hayakawa et al.; Kato et al., Biol. Pharm. Bull. 16:960, 1993.) These formulations were prepared by mixing soybean phosphatidylcholine, α-tocopherol, and ethoxylated hydrogenated castor oil (HCO-60) in methanol, concentrating the mixture under vacuum, and then reconstituting the mixture with water. A liposomal formulation of dipalmitoylphosphatidylcholine (DPPC) with a soybean-derived sterylglucoside mixture (SG) and cholesterol (Ch) has also been shown to target the liver. (See Shimizu et al., Biol. Pharm. Bull. 20:881, 1997.)

Alternatively, various targeting counter-receptors can be bound to the surface of the liposome, such as antibodies, antibody fragments, carbohydrates, vitamins, and transport proteins. For example, for targeting to the liver, liposomes can be modified with branched type galactosyllipid derivatives to target asialoglycoprotein (galactose) receptors, which are exclusively expressed on the surface of liver cells. (See Kato and Sugiyama, Crit. Rev. Ther. Drug Carrier Syst. 14:287, 1997; Murahashi et al., Biol. Pharm. Bull. 20:259, 1997.) In a more general approach to tissue targeting, target cells are prelabeled with biotinylated antibodies specific for a counter-receptor expressed by the target cell. (See Harasym et al., Adv. Drug Deliv. Rev. 32:99, 1998.) After plasma elimination of free antibody, streptavidin-conjugated liposomes are administered. In another approach, targeting antibodies are directly attached to liposomes. (See Harasym et al., supra.)

Polypeptides and antibodies can be encapsulated within liposomes using standard techniques of protein microencapsulation. (See, e.g., Anderson et al., Infect. Immun. 31:1099, 1981; Anderson et al., Cancer Res. 50:1853, 1990; Cohen et al., Biochim. Biophys. Acta 1063:95, 1991; Alving et al. “Preparation and Use of Liposomes in Immunological Studies,” in Liposome Technology (Vol. III) 317 (Gregoriadis, ed., CRC Press, 2nd ed. 1993); Wassef et al., Meth. Enzymol. 149:124, 1987.) As noted above, therapeutically useful liposomes may contain a variety of components. For example, liposomes may comprise lipid derivatives of poly(ethylene glycol). (See Allen et al., Biochim. Biophys. Acta 1150:9, 1993.)

Degradable polymer micro spheres have been designed to maintain high systemic levels of therapeutic proteins. Micro spheres are prepared from degradable polymers such as poly(lactide-co-glycolide) (PLG), polyanhydrides, poly (ortho esters), nonbiodegradable ethylvinyl acetate polymers, in which proteins are entrapped in the polymer. (See, e.g., Gombotz and Pettit, Bioconjugate Chem. 6:332, 1995; Ranade, “Role of Polymers in Drug Delivery,” in Drug Delivery Systems 51-93 (Ranade and Hollinger, eds., CRC Press 1995); Roskos and Maskiewicz, “Degradable Controlled Release Systems Useful for Protein Delivery,” in Protein Delivery: Physical Systems 45-92 (Sanders and Hendren, eds., Plenum Press 1997); Bartus et al., Science 281:1161, 1998; Putney and Burke, Nature Biotechnology 16:153, 1998; Putney, Curr. Opin. Chem. Biol. 2:548, 1998.) Polyethylene glycol (PEG)-coated nanospheres can also provide carriers for intravenous administration of therapeutic proteins. (See, e.g., Gref et al., Pharm. Biotechnol. 10:167, 1997.)

Other dosage forms can be devised by those skilled in the art, as shown by, e.g., Ansel and Popovich, Pharmaceutical Dosage Forms and Drug Delivery Systems (Lea & Febiger, 5th ed. 1990); Gennaro (ed.), Remington's Pharmaceutical Sciences (Mack Publishing Company, 19th ed. 1995), and Ranade and Hollinger, Drug Delivery Systems (CRC Press 1996).

Pharmaceutical compositions as described herein may also be used in the context of combination therapy. The term “combination therapy” is used herein to denote that a subject is administered at least one therapeutically effective dose of a bispecific binding protein comprising a VEGF-A antibody/soluble FGF receptor bispecific binding protein and another therapeutic agent. For example, in the context of cancer immunotherapy, compositions comprising a bispecific binding protein comprising a VEGF-A antibody/soluble FGF receptor bispecific binding protein can be used as an angiogenesis inhibition agent in combination with chemotherapy or radiation. A bispecific binding protein comprising a VEGF-A antibody/soluble FGF receptor bispecific binding protein can work in synergy with conventional types of chemotherapy or radiation. The bispecific binding proteins can further reduce tumor burden and allow more efficient killing by the chemotherapeutic.

Compositions of the present invention demonstrating angiogenesis inhibiting activity can also be used in combination with immunomodulatory compounds including various cytokines and co-stimulatory/inhibitory molecules. These could include, but are not limited to, the use of cytokines that stimulate anti-cancer immune responses. For instance, the combined use of IL-2 and IL-12 shows beneficial effects in T-cell lymphoma, squamous cell carcinoma, and lung cancer. (See Zaki et al., J. Invest. Dermatol. 118:366-71, 2002; Li et al., Arch. Otolaryngol. Head Neck Surg. 127:1319-24, 2001; Hiraki et al., Lung Cancer 35:329-33, 2002.) In addition, VEGF-A antibody/soluble FGF receptor bispecific binding proteins could be combined with reagents that co-stimulate various cell surface molecules found on immune-based effector cells, such as the activation of CD137. (See Wilcox et al., J. Clin. Invest. 109:651-9, 2002) or inhibition of CTLA4 (Chambers et al., Ann. Rev. Immunol. 19:565-94, 2001). Alternatively, a bispecific binding protein comprising a VEGF-A antibody/soluble FGF receptor bispecific binding protein could be used with reagents that induce tumor cell apoptosis by interacting with TRAIL-related receptors. (See, e.g., Takeda et al., J. Exp. Med. 195:161-9, 2002; Srivastava, Neoplasia 3:535-46, 2001.) Such reagents include TRAIL ligand, TRAIL ligand-Ig fusions, anti-TRAIL antibodies, and the like.

In other variations, a bispecific binding protein comprising a VEGF-A antibody/soluble FGF receptor bispecific binding protein is used in combination with a monoclonal antibody therapy that does not specifically target angiogenesis. Such combination therapy is particularly useful for treatment of cancer, in which the use of monoclonal antibodies, particularly antibodies directed against tumor-expressed antigens, is becoming a standard practice for many tumors including breast cell carcinoma (trastuzumab or HERCEPTIN®) and colon carcinoma (cetuximab or ERBITUX®).

Pharmaceutical compositions may be supplied as a kit comprising a container that comprises a therapeutic compositions as described herein. A therapeutic composition can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a therapeutic composition. Such a kit may further comprise written information on indications and usage of the pharmaceutical composition.

B. Cancer Treatment

1. Types of Cancer

Cancers amenable to treatment in accordance with the present invention include cancers characterized by the presence of solid tumors. As previously discussed, the quantity of blood vessels in a tumor tissue is a strong negative prognostic indicator for cancers involving solid tumor formation, (see, e.g., Weidner et al., (1992), supra; Weidner et al., (1993), supra; Li et al., supra; Foss et al., supra), and both the VEGF and FGF family of signaling molecules appear to play key roles in the development of new blood vessels associated with solid tumors. Table 4 below lists some cancers characterized by solid tumor formation, organized predominantly by target tissues.

TABLE 4
Exemplary Cancers Involving Solid Tumor Formation

| | |
|---|---|
| 1. | Head and Neck cancer |

| | |
|---|---|
| a. | Brain |
| b. | Oral cavity |
| c. | Orophyarynx |
| d. | Nasopharynx |
| e. | Hypopharynx |
| f. | Nasal cavities and paranasal sinuses |
| g. | Larynx |
| h. | Lip |

| | |
|---|---|
| 2. | Lung cancers |

| | |
|---|---|
| a. | Non-small cell carcinoma |
| b. | Small cell carcinoma |

| | |
|---|---|
| 3. | Gastrointestinal Tract cancers |

| | |
|---|---|
| a. | Colorectal cancer |
| b. | Gastric cancer |
| c. | Esophageal cancer |
| d. | Anal cancer |
| e. | Extrahepatic Bile Duct cancer |
| f. | Cancer of the Ampulla of Vater |
| g. | Gastrointestinal Stromal Tumor (GIST) |

| | |
|---|---|
| 4. | Liver cancer |

| | |
|---|---|
| a. | Liver Cell Adenoma |
| b. | Hepatocellular Carcinoma |

| | |
|---|---|
| 5. | Breast cancer |
| 6. | Gynecologic cancer |

| | |
|---|---|
| a. | Cervical cancer |
| b. | Ovarian cancer |
| c. | Vaginal cancer |
| d. | Vulvar cancer |
| e. | Gestational Trophoblastic Neoplasia |
| f. | Uterine cancer |

| | |
|---|---|
| 7. | Urinary Tract cancer |

| | |
|---|---|
| a. | Renal cancer carcinoma |
| b. | Prostate cancer |
| c. | Urinary Bladder cancer |
| d. | Penile cancer |
| e. | Urethral cancer |

| | |
|---|---|
| 8. | Urinary Bladder cancer |
| 9. | Neurological Tumors |

| | |
|---|---|
| a. | Astrocytoma and glioblastoma |
| b. | Primary CNS lymphoma |
| c. | Medulloblastoma |
| d. | Germ Cell tumors |
| e. | Retinoblastoma |

| | |
|---|---|
| 10. | Endocrine Neoplasms |

| | |
|---|---|
| a. | Thyroid cancer |
| b. | Pancreatic cancer |

| | |
|---|---|
| 1) | Islet Cell tumors |

| | |
|---|---|
| a) | Insulinomas |
| b) | Glucagonomas |

| | |
|---|---|
| c. | Pheochromocytoma |
| d. | Adrenal carcinoma |
| e. | Carcinoid tumors |
| f. | Parathyroid carcinoma |
| g. | Pineal gland neoplasms |

| | |
|---|---|
| 11. | Skin cancers |

| | |
|---|---|
| a. | Malignant melanoma |
| b. | Squamous Cell carcinoma |
| c. | Basal Cell carcinoma |
| d. | Kaposi's Sarcoma |

| | |
|---|---|
| 12. | Bone cancers |

| | |
|---|---|
| a. | Osteoblastoma |
| b. | Osteochondroma |
| c. | Osteosarcoma |

| | |
|---|---|
| 13. | Connective Tissue neoplasms |

| | |
|---|---|
| a. | Chondroblastoma |
| b. | Chondroma |

| | |
|---|---|
| 14. | Childhood Cancers |

| | |
|---|---|
| a. | Brain cancers |
| b. | Neuroblastoma |
| c. | Wilm's Tumor (nephroblastoma) |
| d. | Phabdomyosarcoma |
| e. | Retinoblastoma |

| | |
|---|---|
| 15. | Immunotherapeutically sensitive cancers |

| | |
|---|---|
| a. | melanoma |
| b. | kidney cancer |
| c. | breast cancer |
| d. | prostate cancer |
| e. | colorectal cancer |
| f. | cervical cancer |
| g. | ovarian cancer |
| h. | lung cancer |

Accordingly, in certain embodiments, a bispecific binding protein comprising a VEGF-A antibody/soluble FGF receptor bispecific binding protein as described herein is used to treat a cancer characterized by the presence of a solid tumor, such as, e.g., any of the cancers listed in Table 4. For example, in some embodiments, the cancer to be treated in accordance with the present invention is selected from the following: a cancer of the head and neck (e.g., a cancer of the oral cavity, orophyarynx, nasopharynx, hypopharynx, nasal cavity or paranasal sinuses, larynx, lip, or salivary gland); a lung cancer (e.g., non-small cell lung cancer, small cell carcinoma, or mesothelimia); a gastrointestinal tract cancer (e.g., colorectal cancer, gastric cancer, esophageal cancer, or anal cancer); gastrointestinal stromal tumor (GIST); pancreatic adenocarcinoma; pancreatic acinar cell carcinoma; a cancer of the small intestine; a cancer of the liver or biliary tree (e.g., liver cell adenoma, hepatocellular carcinoma, hemangiosarcoma, extrahepatic or intrahepatic cholangiosarcoma, cancer of the ampulla of vater, or gallbladder cancer); a breast cancer (e.g., metastatic breast cancer or inflammatory breast cancer); a gynecologic cancer (e.g., cervical cancer, ovarian cancer, fallopian tube cancer, peritoneal carcinoma, vaginal cancer, vulvar cancer, gestational trophoblastic neoplasia, or uterine cancer, including endometrial cancer or uterine sarcoma); a cancer of the urinary tract (e.g., prostate cancer; bladder cancer; penile cancer; urethral cancer, or kidney cancer such as, for example, renal cell carcinoma or transitional cell carcinoma, including renal pelvis and ureter); testicular cancer; a cancer of the central nervous system (CNS) such as an intracranial tumor (e.g., astrocytoma, anaplastic astrocytoma, glioblastoma, oligodendroglioma, anaplastic oligodendroglioma, ependymoma, primary CNS lymphoma, medulloblastoma, germ cell tumor, pineal gland neoplasm, meningioma, pituitary tumor, tumor of the nerve sheath (e.g., schwannoma), chordoma, craniopharyngioma, a chloroid plexus tumor (e.g., chloroid plexus carcinoma); or other intracranial tumor of neuronal or glial origin) or a tumor of the spinal cord (e.g., schwannoma, meningioma); an endocrine neoplasm (e.g., thyroid cancer such as, for example, thyroid carcinoma, medullary cancer, or thyroid lymphoma; a pancreatic endocrine tumor such as, for example, an insulinoma or glucagonoma; an adrenal carcinoma such as, for example, pheochromocytoma; a carcinoid tumor; or a parathyroid carcinoma); a skin cancer (e.g., squamous cell carcinoma; basal cell carcinoma; Kaposi's sarcoma; or a malignant melanoma such as, for example, an intraocular melanoma); a bone cancer (e.g., a bone sarcoma such as, for example, osteosarcoma, osteochondroma, or Ewing's sarcoma); multiple myeloma; a chloroma; a soft tissue sarcoma (e.g., a fibrous tumor or fibrohistiocytic tumor); a tumor of the smooth muscle or skeletal muscle; a blood or lymph vessel perivascular tumor (e.g., Kaposi's sarcoma); a synovial tumor; a mesothelial tumor; a neural tumor; a paraganglionic tumor; an extraskeletal cartilaginous or osseous tumor; and a pluripotential mesenchymal tumor.

In some variations, the cancer to be treated is a childhood cancer such as, for example, brain cancer, neuroblastoma, Wilm's tumor (nephroblastoma), rhabdomyosarcoma, retinoblastoma, or hepatoblastoma.

In other variations, the cancer is an immunotherapeutically sensitive cancer such as, for example, melanoma, kidney cancer, breast cancer, prostate cancer, colorectal cancer, cervical cancer, ovarian cancer, or lung cancer.

Some of the cancers listed above, including some of the relevant animal models for evaluating the effects of VEGF-A antibody/soluble FGF receptor bispecific binding protein on tumor responses, are discussed in further detail below.

a. Prostate Cancer

Prostate cancer is abnormal growth within a gland in the male reproductive system found below the bladder and in front of the rectum. Almost all prostate cancers arise from the secretory glandular cells in the prostate so are therefore prostatic adenocarcinomas. In the United States, cancer of the prostate is a common malignant cancer in men, second only to lung cancer. Carcinoma of the prostate is predominantly a tumor of older men, which frequently responds to treatment when widespread and may be cured when localized. It is estimated that 17% of men will be diagnosed with prostate cancer in their lifetime. The tumors generally originate as small and well-defined lesions, and can often present as multiple primary tumors (Villers et al. 1992). Progression is both local and distant, typically to seminal vesicles, ejaculatory ducts and pelvic lymph nodes and at more advanced stages bone, liver and lungs. Once metastasis has occurred the rate of cancer cell proliferation accelerates

The effects of bispecific binding compositions comprising a soluble FGF receptor and an anti-VEGF-A antibody on tumor response can be evaluated in the mouse models that are available for prostate cancer (reviewed by Ahmad et al., Expert Rev Mol Med, 10:e16 (2008)) and as provided in Example 12.

b. Melanoma

Superficial spreading melanoma is the most common type of melanoma. About 7 out of 10 (70%) are this type. They occur mostly in middle-aged people. The most common place in women is on the legs, while in men it is more common on the trunk, particularly the back. They tend to start by spreading out across the surface of the skin: this is known as the radial growth phase. If the melanoma is removed at this stage there is a very high chance of cure. If the melanoma is not removed, it will start to grow down deeper into the layers of the skin. There is then a risk that it will spread in the bloodstream or lymph system to other parts of the body. Nodular melanoma occurs most often on the chest or back. It is most commonly found in middle-aged people. It tends to grow deeper into the skin quite quickly if it is not removed. This type of melanoma is often raised above the rest of the skin surface and feels like a bump. It may be very dark brown-black or black. Lentigo maligna melanoma is most commonly found on the face, particularly in older people. It grows slowly and may take several years to develop. Acral melanoma is usually found on the palms of the hands, soles of the feet or around the toenails. Other very rare types of melanoma of the skin include amelanotic melanoma (in which the melanoma loses its pigment and appears as a white area) and desmoplastic melanoma (which contains fibrous scar tissue). Malignant melanoma can start in parts of the body other than the skin but this is very rare. The parts of the body that may be affected are the eye, the mouth, under the fingernails (known as subungual melanoma) the vulval or vaginal tissues, or internally.

Most melanomas start with a change in the appearance of normal skin. This can look like an abnormal new mole. Less than a third develop in existing moles. It can be difficult to tell the difference between a mole and a melanoma, but the following checklist can be used to help. It is known as the ABCD list. Asymmetry—Ordinary moles are usually symmetrical in shape. Melanomas are likely to be irregular or asymmetrical. Border—Moles usually have a well-defined regular border. Melanomas are more likely to have an irregular border with jagged edges. Colour—Moles are usually a uniform brown. Melanomas tend to have more than one colour. They may be varying shades of brown mixed with black, red, pink, white or a bluish tint. Diameter—Moles are normally no bigger than the blunt end of a pencil (about 6 mm across). Melanomas are usually more than 7 mm in diameter. Normal moles can be raised up from the skin and/or may be hairy. Itching, crusting or bleeding may also occur in melanomas—these are less common signs but should not be ignored (cancerbacup internet website). The effects of a bispecific binding protein comprising a VEGF-A antibody/soluble FGF receptor bispecific binding protein on tumor response can be evaluated in a murine melanoma model similar to that described in Hermans et al., Cancer Res. 63:8408-13, 2003; Ramont et al., Exp. Cell Res. 29:1-10, 2003; Safwat et al., J. Exp. Ther. Oncol. 3:161-8, 2003; and Fidler, Nat New Biol. 242:148-9, 1973.

c. Renal Cell Carcinoma

Renal cell carcinoma, a form of kidney cancer that involves cancerous changes in the cells of the renal tubule, is the most common type of kidney cancer in adults. Why the cells become cancerous is not known. A history of smoking greatly increases the risk for developing renal cell carcinoma. Some people may also have inherited an increased risk to develop renal cell carcinoma, and a family history of kidney cancer increases the risk. People with von Hippel-Lindau disease, a hereditary disease that affects the capillaries of the brain, commonly also develop renal cell carcinoma. Kidney disorders that require dialysis for treatment also increase the risk for developing renal cell carcinoma. The first symptom is usually blood in the urine. Sometimes both kidneys are involved. The cancer metastasizes or spreads easily, most often to the lungs and other organs, and about one-third of patients have metastasis at the time of diagnosis (Medline Plus Medical Encyclopedia Internet website). The effects of a bispecific binding protein comprising a VEGF-A antibody/soluble FGF receptor bispecific binding protein on tumor response can be evaluated in a murine renal cell carcinoma model similar to that described in Sayers et al., Cancer Res. 50:5414-20, 1990; Salup et al., Immunol. 138:641-7, 1987; and Luan et al., Transplantation 73:1565-72, 2002.

d. Cervical Cancer

The cervix is the neck of the uterus that opens into the vagina. Cervical cancer, also called cervical carcinoma, develops from abnormal cells on the surface of the cervix. Cervical cancer is one of the most common cancers affecting women. Cervical cancer is usually preceded by dysplasia, precancerous changes in the cells on the surface of the cervix. These abnormal cells can progress to invasive cancer. Once the cancer appears it can progress through four stages. The stages are defined by the extent of spread of the cancer. The more the cancer has spread, the more extensive the treatment is likely to be. There are 2 main types of cervical cancer: (1) Squamous type (epidermoid cancer): This is the most common type, accounting for about 80% to 85% of cervical cancers. This cancer may be caused by sexually transmitted diseases. One such sexual disease is the human papillomavirus, which causes venereal warts. The cancerous tumor grows on and into the cervix. This cancer generally starts on the surface of the cervix and may be diagnosed at an early stage by a Pap smear. (2) Adenocarcinoma: This type of cervical cancer develops from the tissue in the cervical glands in the canal of the cervix. Early cervical cancer usually causes no symptoms. The cancer is usually detected by a Pap smear and pelvic exam. This is why you should start having Pap smears and pelvic exams as soon as you become sexually active. Healthy young women who have never been sexually active should have their first annual pelvic exam by age 18. Later stages of cervical cancer cause abnormal vaginal bleeding or a bloodstained discharge at unexpected times, such as between menstrual periods, after intercourse, or after menopause. Abnormal vaginal discharge may be cloudy or bloody or may contain mucus with a bad odor. Advanced stages of the cancer may cause pain (University of Michigan Health System Internet website). The effects of a bispecific binding protein comprising a VEGF-A antibody/soluble FGF receptor bispecific binding protein on tumor response can be evaluated in a murine cervical cancer model similar to that described in Ahn et al., Hum. Gene Ther. 14:1389-99, 2003; Hussain et al., Oncology 49:237-40, 1992; and Sengupta et al., Oncology 48:258-61, 1991.

e. Head and Neck Tumors

Most cancers of the head and neck are of a type called carcinoma (in particular squamous cell carcinoma). Carcinomas of the head and neck start in the cells that form the lining of the mouth, nose, throat or ear, or the surface layer covering the tongue. However, cancers of the head and neck can develop from other types of cells. Lymphoma develops from the cells of the lymphatic system. Sarcoma develops from the supportive cells which make up muscles, cartilage or blood vessels. Melanoma starts from cells called melanocytes, which give colour to the eyes and skin. The symptoms of a head and neck cancer will depend on where it is—for example, cancer of the tongue may cause some slurring of speech. The most common symptoms are an ulcer or sore area in the head or neck that does not heal within a few weeks; difficulty in swallowing, or pain when chewing or swallowing; trouble with breathing or speaking, such as persistent noisy breathing, slurred speech or a hoarse voice; a numb feeling in the mouth; a persistent blocked nose, or nose bleeds; persistent earache, ringing in the ear, or difficulty in hearing; a swelling or lump in the mouth or neck; pain in the face or upper jaw; in people who smoke or chew tobacco, pre-cancerous changes can occur in the lining of the mouth, or on the tongue. These can appear as persistent white patches (leukoplakia) or red patches (erythroplakia). They are usually painless but can sometimes be sore and may bleed (Cancerbacup Internet website). The effects of a bispecific binding protein comprising a VEGF-A antibody/soluble FGF receptor bispecific binding protein on tumor response can be evaluated in a murine head and neck tumor model similar to that described in Kuriakose et al., Head Neck 22:57-63, 2000; Cao et al., Clin. Cancer Res. 5:1925-34, 1999; Hier et al., Laryngoscope 105:1077-80, 1995; Braakhuis et al., Cancer Res. 51:211-4, 1991; Baker, Laryngoscope 95:43-56, 1985; and Dong et al., Cancer Gene Ther. 10:96-104, 2003.

f. Brain Cancer

Tumors that begin in brain tissue are known as primary tumors of the brain. Primary brain tumors are named according to the type of cells or the part of the brain in which they begin. The most common primary brain tumors are gliomas. They begin in glial cells. There are many types of gliomas. (1) Astrocytoma—The tumor arises from star-shaped glial cells called astrocytes. In adults, astrocytomas most often arise in the cerebrum. In children, they occur in the brain stem, the cerebrum, and the cerebellum. A grade III astrocytoma is sometimes called an anaplastic astrocytoma. A grade IV astrocytoma is usually called a glioblastoma multiforme. (2) Brain stem glioma—The tumor occurs in the lowest part of the brain. Brain stem gliomas most often are diagnosed in young children and middle-aged adults. (3) Ependymoma—The tumor arises from cells that line the ventricles or the central canal of the spinal cord. They are most commonly found in children and young adults. (4) Oligodendroglioma—This rare tumor arises from cells that make the fatty substance that covers and protects nerves. These tumors usually occur in the cerebrum. They grow slowly and usually do not spread into surrounding brain tissue. They are most common in middle-aged adults. The symptoms of brain tumors depend on tumor size, type, and location. Symptoms may be caused when a tumor presses on a nerve or damages a certain area of the brain. They also may be caused when the brain swells or fluid builds up within the skull. These are the most common symptoms of brain tumors: Headaches (usually worse in the morning); Nausea or vomiting; Changes in speech, vision, or hearing; Problems balancing or walking; Changes in mood, personality, or ability to concentrate; Problems with memory; Muscle jerking or twitching (seizures or convulsions); and Numbness or tingling in the arms or legs (National Cancer Institute's Internet website). The effects of a bispecific binding protein comprising a VEGF-A antibody/soluble FGF receptor bispecific binding protein on tumor response can be evaluated in a glioma animal model similar to that described in Schueneman et al., Cancer Res. 63:4009-16, 2003; Martinet et al., Eur. J. Surg. Oncol. 29:351-7, 2003; Bello et al., Clin. Cancer Res. 8:3539-48, 2002; Ishikawa et al., Cancer Sci. 95:98-103, 2004; Degen et al., J. Neurosurg. 99:893-8, 2003; Engelhard et al., Neurosurgery 48:616-24, 2001; Watanabe et al., Neurol. Res. 24:485-90, 2002; and Lumniczky et al., Cancer Gene Ther. 9:44-52, 2002.

g. Thyroid Cancer

Papillary and follicular thyroid cancers account for 80 to 90 percent of all thyroid cancers. Both types begin in the follicular cells of the thyroid. Most papillary and follicular thyroid cancers tend to grow slowly. If they are detected early, most can be treated successfully. Medullary thyroid cancer accounts for 5 to 10 percent of thyroid cancer cases. It arises in C cells, not follicular cells. Medullary thyroid cancer is easier to control if it is found and treated before it spreads to other parts of the body. Anaplastic thyroid cancer is the least common type of thyroid cancer (only 1 to 2 percent of cases). It arises in the follicular cells. The cancer cells are highly abnormal and difficult to recognize. This type of cancer is usually very hard to control because the cancer cells tend to grow and spread very quickly. Early thyroid cancer often does not cause symptoms. But as the cancer grows, symptoms may include: A lump, or nodule, in the front of the neck near the Adam's apple; Hoarseness or difficulty speaking in a normal voice; Swollen lymph nodes, especially in the neck; Difficulty swallowing or breathing; or Pain in the throat or neck (National Cancer Institute's Internet website). The effects of a bispecific binding protein comprising a VEGF-A antibody/soluble FGF receptor bispecific binding protein on tumor response can be evaluated in a murine or rat thyroid tumor model similar to that described in Quidville et al., Endocrinology 145:2561-71, 2004 (mouse model); Cranston et al., Cancer Res. 63:4777-80, 2003 (mouse model); Zhang et al., Clin Endocrinol (Oxf). 52:687-94, 2000 (rat model); and Zhang et al., Endocrinology 140:2152-8, 1999 (rat model).

h. Liver Cancer

There are two different types of primary liver cancer. The most common kind is called hepatoma or hepatocellular carcinoma (HCC), and arises from the main cells of the liver (the hepatocytes). This type is usually confined to the liver, although occasionally it spreads to other organs. It occurs mostly in people with a liver disease called cirrhosis. There is also a rarer sub-type of hepatoma called Fibrolamellar hepatoma, which may occur in younger people and is not related to previous liver disease. The other type of primary liver cancer is called cholangiocarcinoma or bile duct cancer, because it starts in the cells lining the bile ducts. Most people who develop hepatoma usually also have a condition called cirrhosis of the liver. This is a fine scarring throughout the liver which is due to a variety of causes including infection and heavy alcohol drinking over a long period of time. However, only a small proportion of people who have cirrhosis of the liver develop primary liver cancer. Infection with either the hepatitis B or hepatitis C virus can lead to liver cancer, and can also be the cause of cirrhosis, which increases the risk of developing hepatoma. People who have a rare condition called haemochromatosis, which causes excess deposits of iron in the body, have a higher chance of developing hepatoma. Thus, the bispecific binding protein comprising a VEGF-A antibody/soluble FGF receptor bispecific binding protein of the present invention may be used to treat, prevent, inhibit the progression of, delay the onset of, and/or reduce the severity or inhibit at least one of the conditions or symptoms associated with hepatocellular carcinoma. The hepatocellular carcinoma may or may not be associated with an hepatitis (e.g., hepatitis A, hepatitis B, hepatitis C and hepatitis D) infection.

The effects of a bispecific binding protein comprising a VEGF-A antibody/soluble FGF receptor bispecific binding protein on tumor response can be evaluated in a hepatocellular carcinoma transgenic mouse model, which includes the overexpression of transforming growth factor-α (TFG-α) alone (Jhappan et al., Cell, 61:1137-1146, 1990; Sandgren et al., Mol. Cell. Biol., 13:320-330, 1993; Sandgren et al., Oncogene 4:715-724, 1989; and Lee et al., Cancer Res. 52:5162:5170, 1992) or in combination with c-myc (Murakami et al., Cancer Res., 53:1719-1723, 1993), mutated H-ras (Saitoh et al., Oncogene 5:1195-2000, 1990), hepatitis B viral genes encoding HbsAg and HBx (Toshkov et al., Hepatology 20:1162-1172, 1994; Koike et al., Hepatology 19:810-819, 1994), SV40 large T antigen (Sepulveda et al., Cancer Res. 49:6108-6117, 1989; Schirmacher et al., Am. J. Pathol., 139:231-241, 1991) and FGF19 (Nicholes et al., American Journal of Pathology, 160:2295-2307, 2002).

i. Lung Cancer

The effects of a bispecific binding protein comprising a VEGF-A antibody/soluble FGF receptor bispecific binding protein on tumor response can be evaluated in a human small/non-small cell lung carcinoma xenograft model. Briefly, human tumors are grafted into immunodeficient mice and these mice are treated with a bispecific binding protein comprising a VEGF-A antibody/soluble FGF receptor bispecific binding protein alone or in combination with other agents which can be used to demonstrate the efficacy of the treatment by evaluating tumor growth (Nemati et al., Clin Cancer Res. 6:2075-86, 2000; and Hu et al., Clin. Cancer Res. 10:7662-70, 2004).

2. Endpoints and Anti-Tumor Activity for Solid Tumors

While each protocol may define tumor response assessments differently, the RECIST (Response evaluation Criteria in solid tumors) criteria is currently considered to be the recommended guidelines for assessment of tumor response by the National Cancer Institute (see Therasse et al., J. Natl. Cancer Inst. 92:205-216, 2000). According to the RECIST criteria tumor response means a reduction or elimination of all measurable lesions or metastases. Disease is generally considered measurable if it comprises lesions that can be accurately measured in at least one dimension as ≧20 mm with conventional techniques or ≧10 mm with spiral CT scan with clearly defined margins by medical photograph or X-ray, computerized axial tomography (CT), magnetic resonance imaging (MRI), or clinical examination (if lesions are superficial). Non-measurable disease means the disease comprises of lesions <20 mm with conventional techniques or <10 mm with spiral CT scan, and truly non-measurable lesions (too small to accurately measure). Non-measurable disease includes pleural effusions, ascites, and disease documented by indirect evidence.

The criteria for objective status are required for protocols to assess solid tumor response. Representative criteria include the following: (1) Complete Response (CR), defined as complete disappearance of all measurable disease; no new lesions; no disease related symptoms; no evidence of non-measurable disease; (2) Partial Response (PR) defined as 30% decrease in the sum of the longest diameter of target lesions (3) Progressive Disease (PD), defined as 20% increase in the sum of the longest diameter of target lesions or appearance of any new lesion; (4) Stable or No Response, defined as not qualifying for CR, PR, or Progressive Disease. (See Therasse et al., supra.)

Additional endpoints that are accepted within the oncology art include overall survival (OS), disease-free survival (DFS), objective response rate (ORR), time to progression (TTP), and progression-free survival (PFS) (see Guidance for Industry: Clinical Trial Endpoints for the Approval of Cancer Drugs and Biologics, April 2005, Center for Drug Evaluation and Research, FDA, Rockville, Md.)

3. Combination Cancer Therapy

As previously discussed, in certain embodiments, a bispecific VEGF-A antibody/FGFR soluble receptor combination is used in combination with a second agent for treatment of a neovascular disorder. When used for treating cancer, antagonists of the present invention may be used in combination with conventional cancer therapies such as, e.g., surgery, radiotherapy, chemotherapy, or combinations thereof. In certain aspects, other therapeutic agents useful for combination cancer therapy with a bispecific binding protein comprising a VEGF-A antibody/soluble FGF receptor bispecific binding protein include other anti-angiogenic agents. In some other aspects, other therapeutic agents useful for combination therapy with a bispecific binding protein comprising a VEGF-A antibody/soluble FGF receptor bispecific binding protein include an antagonist of other factors that are involved in tumor growth such as, for example, EGFR, ErbB2 (Her2), ErbB3, ErbB4, or TNF. In some aspects, a bispecific binding protein comprising a VEGF-A antibody/soluble FGF receptor bispecific binding protein is co-administered with a cytokine (e.g., a cytokine that stimulates an immune response against a tumor). Exemplary combination therapies particularly amenable for treatment of cancer are described in further detail below.

a. Antibodies Targeting Tumor-Associated Antigens in Combination with Bispecific Binding Proteins Comprising Bispecific Antibody/Soluble Receptor Binding Proteins

Antibody therapy has been particularly successful in cancer treatment because certain tumors either display unique antigens, lineage-specific antigens, or antigens present in excess amounts relative to normal cells. One of the mechanisms associated with the anti-tumor activity of monoclonal antibody therapy is antibody dependent cellular cytotoxicity (ADCC). In ADCC, monoclonal antibodies bind to a target cell (e.g., cancer cell) and specific effector cells expressing receptors for the monoclonal antibody (e.g., NK cells, monocytes, granulocytes) bind the monoclonal antibody/target cell complex resulting in target cell death. In certain variations of the present invention, a bispecific binding protein comprising a VEGF-A antibody/soluble FGF receptor bispecific binding protein is co-administered with a monoclonal antibody against a tumor-associated antigen. The dose and schedule of the MAbs is based on pharmacokinetic and toxicokinetic properties ascribed to the specific antibody co-administered, and should optimize these effects, while minimizing any toxicity that may be associated with administration of a bispecific binding protein comprising a VEGF-A antibody/soluble FGF receptor bispecific binding protein.

Combination therapy with a bispecific binding protein comprising a VEGF-A antibody/soluble FGF receptor bispecific binding protein and a monoclonal antibody against a tumor-associated antigen may be indicated when a first line treatment has failed and may be considered as a second line treatment. The present invention also provides using the combination as a first line treatment in patient populations that are newly diagnosed and have not been previously treated with anticancer agents (“de novo patients”) and patients that have not previously received any monoclonal antibody therapy (“naïve patients”).

A bispecific binding protein is also useful in combination therapy with monoclonal antibodies against tumor-associated antigens in the absence of any direct antibody-mediated ADCC of tumor cells. For example, antibodies that block an inhibitory signal in the immune system can lead to augmented immune responses. Examples include (1) antibodies against molecules of the B7R family that have inhibitory function such as, cytotoxic T lymphocyte-associated antigen 4 (CTLA-4), programmed death-1 (PD-1), B and T lymphocyte attenuator (BTLA); (2) antibodies against inhibitory cytokines like IL-10, TGFβ; and (3) antibodies that deplete or inhibit functions of suppressive cells like anti-CD25 or CTLA-4. For example, anti-CTLA4 MAbs in both mice and humans are thought to either suppress function of immune-suppressive regulatory T cells (Tregs) or inhibit the inhibitory signal transmitted through binding of CTLA-4 on T cells to B7-1 or B7-2 molecules on APCs or tumor cells.

Table 8 is a non-exclusive list of monoclonal antibodies approved or being tested for which combination therapy with a bispecific binding protein comprising a VEGF-A antibody/soluble FGF receptor bispecific binding protein is possible.

TABLE 8
Monoclonal Antibody Therapies for Use in Combination with VEGF-A
antibody/soluble FGF receptor bispecific binding proteins

| | | Clinical | |
|---|---|---|---|
| Target | Drug Name | Indication | Company |
| TRAIL-R1 | HGS-ETR1 | cancers | HGS |
| TRAIL-R2 | HGS-ETR2 | solid tumors | HGS |
| CD40 | SGN40 | MM | Seattle Genetics |
| HER2 | Herceptin | Breast cancer | Genentech |
| EGF-R | ABX-EGF | CRC, NSCLC, | Abgenix |
| | | RCC | |
| EGF-R | EMD72000 | solid tumors | Merck |
| EGF-R | MDX-214 | EGF-R-positive | Medarex |
| | | tumors | |
| EGF-R | Erbitux | CRC | Imclone |
| α5β3 integrin | Vitaxin | psoriasis, prostate | AME/Lilly |
| | | cancer | |
| CD152 | CTLA-4 | cancers | Medarex |
| CD49e | Integrin α5 | cancers | Protein Design |
| | | | Labs |
| MUC18 | ABX-MA1 | melanoma | |
| (TIM-like) | | | |
| TAG-72 Mucin | Anatumomab | cancers | |
| CD3 | Ecromeximab | melanoma | Kyowa Hakko |
| CD64 (Fc GR1) | AntiCD64 | cancers | Medarex |
| CEA | CEA-Cide | cancers | Immunomedics |
| EpCAM | Panorex | colorectal cancer | Centocor |
| Lewis-Y-Ag | SGN15 | cancers | Seattle Genetics |

b. A Bispecific Binding Protein Comprising a VEGF-A Antibody/Soluble FGF Receptor Bispecific Binding Protein

In some embodiments, a bispecific binding protein comprising a VEGF-A antibody/soluble FGF receptor bispecific binding protein as described herein is used in combination with a tyrosine kinase inhibitor. Tyrosine kinases are enzymes that catalyze the transfer of the γ phosphate group from the adenosine triphosphate to target proteins. Tyrosine kinases can be classified as receptor and nonreceptor protein tyrosine kinases. They play an essential role in diverse normal cellular processes, including activation through growth receptors and affect proliferation, survival and growth of various cell types. Additionally, they are thought to promote tumor cell proliferation, induce anti-apoptotic effects and promote angiogenesis and metastasis. In addition to activation through growth factors, protein kinase activation through somatic mutation is a common mechanism of tumorigenesis. Some of the mutations identified are in B-Raf kinase, FLt3 kinase, BCR-ABL kinase, c-KIT kinase, epidermal growth factor (EGFR) and PDGFR pathways. The Her2, VEGFR and c-Met are other significant receptor tyrosine kinase (RTK) pathways implicated in cancer progression and tumorigenesis. Because a large number of cellular processes are initiated by tyrosine kinases, they have been identified as key targets for inhibitors.

Tyrosine kinase inhibitors (TKIs) are small molecules that act inside the cell, competing with adenosine triphosphate (ATP) for binding to the catalytic tyrosine kinase domain of both receptor and non-receptor tyrosine kinases. This competitive binding blocks initiation of downstream signaling leading to effector functions associated with these signaling events like growth, survival, and angiogenesis. Using a structure and computational approach, a number of compounds from numerous medicinal chemistry combinatorial libraries was identified that inhibit tyrosine kinases.

Most TKIs are thought to inhibit growth of tumors through direct inhibition of the tumor cell or through inhibition of angiogenesis. Moreover, certain TKIs affect signaling through the VEGF family receptors, including sorafenib and sunitinib. In some cases TKIs have been shown to activate functions of dendritic cells and other innate immune cells, like NK cells. This has been recently reported in animal models for imatinib. Imatinib is a TKI that has shown to enhance killer activity by dendritic cells and NK cells (for review, see Smyth et al., NEJM 354:2282, 2006).

BAY 43-9006 (sorafenib, Nexavar®) and SU11248 (sunitinib, Sutent®) are two such TKIs that have been recently approved for use in metastatic renal cell carcinoma (RCC). A number of other TKIs are in late and early stage development for treatment of various types of cancer. Other TKIs include, but are not limited to: Imatinib mesylate (Gleevec®, Novartis); Gefitinib (Iressa®, AstraZeneca); Erlotinib hydrochloride (Tarceva®, Genentech); Vandetanib (Zactima®, AstraZeneca), Tipifarnib (Zarnestra®, Janssen-Cilag); Dasatinib (Sprycel®, Bristol Myers Squibb); Lonafarnib (Sarasar®, Schering Plough); Vatalanib succinate (Novartis, Schering AG); Lapatinib (Tykerb®, GlaxoSmithKline); Nilotinib (Novartis); Lestaurtinib (Cephalon); Pazopanib hydrochloride (GlaxoSmithKline); Axitinib (Pfizer); Canertinib dihydrochloride (Pfizer); Pelitinib (National Cancer Institute, Wyeth); Tandutinib (Millennium); Bosutinib (Wyeth); Semaxanib (Sugen, Taiho); AZD-2171 (AstraZeneca); VX-680 (Merck, Vertex); EXEL-0999 (Exelixis); ARRY-142886 (Array BioPharma, AstraZeneca); PD-0325901 (Pfizer); AMG-706 (Amgen); BIBF-1120 (Boehringer Ingelheim); SU-6668 (Taiho); CP-547632 (OSI); (AEE-788 (Novartis); BMS-582664 (Bristol-Myers Squibb); JNK-401 (Celgene); R-788 (Rigel); AZD-1152 HQPA (AstraZeneca); NM-3 (Genzyme Oncology); CP-868596 (Pfizer); BMS-599626 (Bristol-Myers Squibb); PTC-299 (PTC Therapeutics); ABT-869 (Abbott); EXEL-2880 (Exelixis); AG-024322 (Pfizer); XL-820 (Exelixis); OSI-930 (OSI); XL-184 (Exelixis); KRN-951 (Kirin Brewery); CP-724714 (OSI); E-7080 (Eisai); HKI-272 (Wyeth); CHIR-258 (Chiron); ZK-304709 (Schering AG); EXEL-7647 (Exelixis); BAY-57-9352 (Bayer); BIBW-2992 (Boehringer Ingelheim); AV-412 (AVEO); YN-968D1 (Advenchen Laboratories); Midostaurin (Novartis); Perifosine (AEterna Zentaris, Keryx, National Cancer Institute); AG-024322 (Pfizer); AZD-1152 (AstraZeneca); ON-01910Na (Onconova); and AZD-0530 (AstraZeneca).

c. Chemotherapy Combinations

In certain embodiments, a bispecific binding protein comprising a VEGF-A antibody/soluble FGF receptor is administered in combination with one or more chemotherapeutic agents. Chemotherapeutic agents have different modes of actions, for example, by influencing either DNA or RNA and interfering with cell cycle replication. Examples of chemotherapeutic agents that act at the DNA level or on the RNA level are anti-metabolites (such as Azathioprine, Cytarabine, Fludarabine phosphate, Fludarabine, Gemcitabine, cytarabine, Cladribine, capecitabine 6-mercaptopurine, 6-thioguanine, methotrexate, 5-fluoroouracil and hyroxyurea); alkylating agents (such as Melphalan, Busulfan, Cis-platin, Carboplatin, Cyclophosphamide, Ifosphamide, Dacarabazine, Procarbazine, Chlorambucil, Thiotepa, Lomustine, Temozolamide); anti-mitotic agents (such as Vinorelbine, Vincristine, Vinblastine, Docetaxel, Paclitaxel); topoisomerase inhibitors (such as Doxorubincin, Amsacrine, Irinotecan, Daunorubicin, Epirubicin, Mitomycin, Mitoxantrone, Idarubicin, Teniposide, Etoposide, Topotecan); antibiotics (such as actinomycin and bleomycin); asparaginase; anthracyclines or taxanes.

d. Radiotherapy Combinations

In some variations, a bispecific binding protein comprising a VEGF-A antibody/soluble FGF receptor bispecific binding protein is administered in combination with radiotherapy. Certain tumors can be treated with radiation or radiopharmaceuticals. Radiation therapy is generally used to treat unresectable or inoperable tumors and/or tumor metastases. Radiotherapy is typically delivered in three ways. External beam irradiation is administered at distance from the body and includes gamma rays (⁶⁰Co)) and X-rays. Brachytherapy uses sources, for example ⁶⁰Co, ¹³⁷Cs, ¹⁹²Ir, or ¹²⁵I, with or in contact with a target tissue.

e. Hormonal Agent Combinations

In some embodiments, a bispecific binding protein comprising a VEGF-A antibody/soluble FGF receptor bispecific binding protein is administered in combination with a hormone or anti-hormone. Certain cancers are associated with hormonal dependency and include, for example, ovarian cancer, breast cancer, and prostate cancer. Hormonal-dependent cancer treatment may comprise use of anti-androgen or anti-estrogen compounds. Hormones and anti-hormones used in cancer therapy include Estramustine phosphate, Polyestradiol phosphate, Estradiol, Anastrozole, Exemestane, Letrozole, Tamoxifen, Megestrol acetate, Medroxyprogesterone acetate, Octreotide, Cyproterone acetate, Bicaltumide, Flutamide, Tritorelin, Leuprorelin, Buserelin and Goserelin.

The invention is further illustrated by the following non-limiting examples.

### Example 1

### Panning for Antibodies that Bind VEGF-A

Antibodies that bind to VEGF-A were identified by screening the Dyax Fab 310 phage library (Dyax Corp., Cambridge, Mass.). The chosen method for selection and screening of the phage-antibody libraries utilized polystyrene immunotubes (NUNC, Denmark) coated with antigen (VEGF-A₁₆₅, R&D Systems). The antibodies were isolated by increasing the stringency after a few rounds of selection. The first generation of antibodies was in the Fab format. The soluble Fab antibodies were generated by MluI (#R0198S, New England Biolabs, Beverly, Mass.) enzyme digestion to remove the geneIII stump from M13 phage. The same strategy of selection, screening, and solubilizing was applied for antibodies in the scFv format.

### Example 2

### Identification of VEGF-A-binding Fab Clones

Fab clones binding VEGF-A were identified by a plate based binding assay. Costar (#9018) 96-well plates were coated with 50 μl VEGF-A (R&D Systems) or PDGF-D (SEQ ID NO:80) homodimer at 0.6 μg/ml in 0.1M NaHCO₃, pH 9.6 overnight at 4° C. The next day, plates were washed three times with 0.1% Tween-20/PBS (PBST). Each well was filled with 100 μl of 2% milk (#170-6404, Bio-Rad)/PBST for one hour at RT for blocking. Assay plates were then washed three times with PBST. Each well was filled with 25 ul of 2% milk/PBST, followed by the addition of 25 ul of Fab supernatant. Wells were then mixed and incubated for one hour at RT. Plates were washed three times with PBST. For Fab detection, 50 ul of (1:4000) anti-Human Fab specific pAb-HRP (#31482, Pierce) in 2% milk/PBST was added to each well for one hour at RT. Plates were then washed three times with PBST. 50 ul of TMB (TMBW-1000-01, BioFX Laboratories) was added to each well to develop for 15 min, followed by the addition of 50 ul of stop buffer (STPR-1000-01, BioFX Laboratories) to quench the reaction. Plates were then read at 450 nm on a plate reader.

### Example 3

### Conversion of VEGF-A-Binding sFab into ScFv

Lambda, kappa, and heavy chain variable regions were amplified from a pool of round 2, Arm A and Arm B VEGF-A-panned Fab Dyax phage DNA in a 3 step process using primers directed against framework sequences for each subtype. The first round PCR amplifies each of the variable framework regions and adds appropriate overhangs to facilitate round 2 PCR reactions. Round 2 PCR reactions add appropriate gly/ser linker sequences to the ends of the proper round 1 PCR products and round 3 PCR reactions overlap the variable light chain lambda, variable light chain kappa, and variable heavy chain products to create scFv products in both LH and HL orientations, which were then cloned into ApaLI/NotI-digested PIMD21 phage display vector.

### Example 4

### Identification sFabs and scFvs that Inhibit VEGF Binding to sVEGFR2

VEGF-A Fab and scFv clones were screened by a plate-based neutralization assay. Costar (#9018) 96-well plates were coated with 100 μl of anti-human IgG Fcγ-specific antibody (#109-005-098, Jackson Immunology) at 1 mg/ml in 0.1M NaHCO₃, pH 9.6 overnight at 4° C. The next day, plates were washed three times with 400 ul 0.1% Tween-20/PBS (PBST). Each well was filled with 100 μl of 1% BSA (#A3059-100G, SIGMA)/PBST for one hour at room temperature (RT) for blocking. Plates were washed three times with PBST. 100 μl of VEGFR2-Fc (SEQ ID NO:81) at 0.2 μg/ml in 1% BSA/PBST was added to each well for one hour at room temperature. Concurrently, in a separate 96 well plate (Costar 3357), 65 μl of Fab or scFv supernatant was added to 65 μl of biotinylated VEGF-A in 1% BSA/PBST at 20 ng/ml for 1 hr at room temperature. Blocked assay plates were washed three times with PBST. Each well was filled with 100 μl of supernatant/biotinylated VEGF-A complex for 1 hr at room temperature. Plates were washed three times with PBST. 100 ml of (1:4000) Streptavidin-HRP (#21124, Pierce) in 1% BSA/PBST was added to each well for one hour at room temperature. Plates were then washed three times with PBST. 100 μl of TMB (TMBW-1000-01, BioFX Laboratories) was added to each well to develop for 20 minutes, followed by the addition of 100 μl of stop buffer (STPR-1000-01, BioFX Laboratories) to quench the reaction. Plates were then read at 450 nm on a plate reader.

### Example 5

### Measurement of Dissociation Rate Constants of Interaction of Human VEGF-A Antagonists with Human VEGF-A Via Surface Plasmon Resonance (Biacore)

Human VEGF-A antagonists were evaluated for their binding affinity to human VEGF-A.

VEGF-A according to their dissociation rate constants using surface plasmon resonance. Dissociation rate constants were measured for the interaction of VEGF-A antagonists with VEGF-A via surface plasmon resonance. The dissociation rate constant (k_{d} (s₋₁)) is a value that reflects the stability of this complex. It is independent of the concentration and therefore suitable for screening and ranking samples with unknown concentrations.

Materials and Methods: A series of experiments were completed to measure the binding affinity of VEGF-A antagonists to VEGF-A. Binding kinetics and affinity studies were performed on a Biacore T-100™ system (GE Healthcare, Piscataway, N.J.). Methods were programmed using Biacore T100™ Control Software, v 1.1.1. Human VEGF-A was covalently immobilized on a CM5 sensor chip using amine coupling chemistry (EDC:NHS) to a density of approximately 200 RU. VEGF-A was immobilized only to the active flow cell. After the immobilization procedure, remaining active sites on the flow cell were blocked with ethanolamine. Non-specifically bound protein was removed by washing with 50 mM NaOH. A reference cell was activated and then blocked with ethanolamine.

The VEGF-A antagonist supernatants (selected from a Dyax phage library screening) were diluted 1:3 in running buffer, injected over the surface and allowed to specifically bind to VEGF-A on a sensor chip with an association time of 5 minutes and dissociation time of 5 minutes. Duplicate injections of 100 nM VEGFR-2-Fc5 and 100 nM anti-VEGF-A monoclonal antibody (Avastin™ Genentech) were used as positive controls. Kinetic binding studies were performed using a flow rate of 30 ul/min. All binding experiments were performed at 25° C. in running buffer of 10 mM HEPES, 150 mM NaCl, 3 mM EDTA 0.05% Surfactant P20, 1 mg/ml bovine serum albumin, pH 7.4. Buffer injections were also performed to allow for subtraction of instrument noise and drift. Between cycles, the flow cell was washed with 10 mM Glycine, pH 1.5 to remove bound VEGF-A antagonists from the surface.

Data was compiled using Biacore T100™ Evaluation software (version 1.1.1). Data was processed by subtracting reference flow cell and blank injections. Baseline stability was assessed to ensure that the regeneration step provided a consistent binding surface throughout the sequence of injections. Since the starting concentrations of the VEGF-A antagonists were unknown, resulting binding curves were globally fit to a 1:1 dissociation binding model to calculate the dissociation rate constants (k_{d} (s₋₁)).

Results: Dissociation rate analysis of VEGF-A antagonists to human VEGF-A was determined Resulting binding curves fit well to a 1:1 dissociation model. The starting concentrations of the VEGF-A antagonists were unknown, therefore only dissociation rate constants (k_{d} (s₋₁)) were reported since k_{d} is independent of concentration. Calculated dissociation rate constants were ranked from slowest to fastest. Under these assay conditions, the VEGF-A antagonists display a large range of dissociation rate constants (1.E₋₅-2.E₋₂ (s₋₁)) for their interaction to VEGF-A (see Table 5). For comparison, the k_{d} of VEGFR-2-Fc5-VEGF-A interaction was approximately 2.E₋₄ s₋₁ and anti-VEGF-A monoclonal antibody Avastin™ Fab-VEGF-A interaction was approximately 8.E₋₅ s₋₁.

TABLE 5
Dissociation Rate Constants of Interaction of VEGF-A
Antagonists with VEGF-A scFvs and controls

| | Off rate [kd (s-1)] |
|---|---|
| Avastin ™ | 1.E 04 |
| Lucentis ™ | 2.E 05 |
| Avastin ™ Fab | 8.E 05 |
| c1094.1_1 | 4.E 04 |
| c1039.1_1 | 2.E 04 |
| c870.1_1 | 2.E 04 |

### Example 6

### Purification of Proteins Derived from E. Coli Periplasmic Fraction

scFv, tandem scFv, and sFab proteins were expressed in the periplasmic space of E. coli cells. Scale of ferment ranged from 25 mL shake flask cultures to 2 L batch fed systems. E. coli cells were spun down using a centrifuge into a pellet. Wet cell pellet was completely re-suspended in periplasting buffer [0.2M Tris, 20% (w/v) sucrose, Complete EDTA-free protease inhibitor cocktail (Roche) pH 7.5] at a ratio of 2 mL per gram of wet cell weight. Lysozyme, an enzyme that facilitates the degradation of the cell wall may or may not be included in the procedure. To determine whether or not to use lysozyme, 500 uL of re-suspended pellet was transferred to an eppendorf tube and 30 U of Ready-Lyse lysozyme (Epicentre) per uL of periplasting buffer used was added and the suspension incubated at room temperature for 5 minutes. After the incubation, the solution was checked for increased viscosity by inversion. If the solution clings to the wall of the tube, then premature cell lysis may be occurring, and the lysozyme is not included in the preparative solution. If the solution does not cling to the tube wall, then the lysozyme is included in the preparative solution. If using lysozyme, 30 U of Ready-Lyse lysozyme (Epicentre) per uL of periplasting buffer used was added and the suspension incubated at room temperature for 4-6 minutes. Ice cold water was added at a ratio of 3 mL per gram of original wet cell pellet weight and the solution incubated for at least 10 minutes but no longer than 30 minutes. The remaining spheroplasts were pelleted via centrifugation at 15,000×g (or 10,000-20,000 RPM, whichever is faster) for at least 15 minutes, but no longer than 45 minutes, at room temperature. The supernatant containing the periplasmic fraction was poured into a new vessel and adjusted to 25 mM Imidazole, 500 mM NaCl using weighed out solid. This solution was filtered through a 0.22 um filter prior to purification using a bottle top filter (Nalgene).

### Immobilized Metal Affinity Chromatography (IMAC) Capture

Traditionally, a 5 mL HisTrap HP column (GE Healthcare) was used for the IMAC step, however, the column size can be scaled up or down depending on the amount of scFv target in the periplasmic fraction as determined by an analytical IMAC-SEC assay. Binding capacity of this IMAC resin has been shown to be at least 20 mg/mL of packed bed. If using columns larger than 10 mL in size, Waters Glass Columns (Millipore) with a 2 and 5 cm internal diameter were preferred. Using an appropriate chromatography station (Akta Explorer using UNICORN software 4.1 and higher [GE Healthcare] or BioCAD Sprint, 700E, or Vision using Perfusion Chromatography software version 3.00 or higher [Applied Biosystems]), the IMAC column was equilibrated in 50 mM NaPO₄, 500 mM NaCl, 25 mM Imidazole pH 7.5 and the periplasmic fraction loaded over it at no faster than 190 cm/hr until depleted. Column was washed with equilibration buffer until monitors at UV A254 nm and UV A280 nm are baseline stable for at least 2 CV at a flow rate not to exceed 190 cm/hr. Bound protein was eluted competitively using 50 mM NaPO₄, 500 mM NaCl, 400 mM Imidazole, pH 7.5 at no faster than 190 cm/hr. Elution fractions were assessed for protein content via UV @A280 nm, analytical size exclusion chromatography, and SDS-PAGE.

### Other Chormatographic Techniques

Purity of the IMAC pool was assessed by SDS-PAGE gel and analytical size exclusion chromatography (SEC). If the pool was not amenable to final clean up via SEC, other chromatographic techniques were employed to further purify the target scFv protein from residual host cell contaminants and aggregates. These conventional techniques included anion exchange, cation exchange, and hydrophobic interaction. Other affinity based approaches were also used, including, but not limited to: utilization of the c-terminal myc tag via anti-myc resin or ligand based affinity approaches using the appropriate ligand covalently coupled to a rigid bead. The utility of these other techniques were determined on a protein to protein basis.

Size Exclusion Chromatography (SEC)

Amount of protein as assessed by UV @A280 nm and analytical SEC method determined the size of gel filtration column used: <1 mg=10/300 Superdex 200 GL column, 1-10 mg=16/60 Superdex 200, >10 mg=26/60 Superdex 200 (All GE Healthcare). IMAC elution pool was concentrated using 10 kD MWCO Ultracel centrifugal concentrator (Millipore) with the final concentrate volume being no more than 3% of the volume of gel filtration column used. Concentrate was injected onto column and the protein eluted isocratically at a flow rate not to exceed 76 cm/hr and no slower than 34 cm/hr. Elution fractions were analyzed by SDS-PAGE, and the appropriate pool made.

### Endotoxin Removal

Final product specifications regarding endotoxin levels were determined by status of a particular cluster. SEC pool was concentrated to >0.25 mg/mL as determined by UV @A280 nm using a 10 kD MWCO Ultracel centrifugal concentrator (Millipore). A Mustang E 0.22 um filter (PALL) was pre-wetted with SEC mobile phase buffer and the SEC concentrate filtered through it via manual syringe delivery system at a flow rate of ˜1 mL/min. Final filtered product was assayed for endotoxin using PTS EndoSafe system (Charles River), concentration via UV @A280 nm, and aliquoted for storage.

### Example 7

### Identification of Neutralizing Anti-human VEGF scFvs Using the 293/KDR/KZ136/c22 VEGF-A-induced Cell-Based Luciferase Assay

To screen candidate molecules (scFv's) for their ability to neutralize the activity of human VEGF-A, a cell-based luciferase assay was performed. 293/KDR/KZ136/c22 cells were plated at a seeding density of 10,000 cells per well in 96-well opaque white tissue-culture treated plates (Costar #3917) in 100 μl complete medium (DMEM, 10% fetal bovine serum (FBS), 1× Sodium Pyruvate, 1× GlutaMax (Invitrogen)) and incubated 48 hours in a 37° C. humidified 5% CO₂ incubator. After 48 hours, complete medium was removed by vacuum aspiration and replaced with 100 μl serum-free medium (DMEM, 1× Sodium Pyruvate, 1× GlutaMax (Invitrogen)) and incubated overnight.

The following day, candidate VEGF-A neutralizing molecules (scFv's, Fabs), positive controls (bevacizumab (anti-VEGF-A monoclonal antibody, Genentech), ranibizumab (anti-VEGF-A affinity-matured Fab, Genentech), and bevacizumab Fab generated in-house) were serially diluted from 200 nM down to 12 pM at 1:5 dilutions along with a non-neutralizer (medium only) in serumfree medium. To these, and equal volume of VEGF-A₁₆₅ was added at 0.54 nM for a final concentration of 0.26 nM VEGF-A and 100 nM to 6 pM neutralizing molecule or positive control. These were incubated for 60 minutes at 37° C. Following incubation, medium was aspirated off the serum-starved cells and 100 μl of the above complexes were added and incubated at 37° C. for 4 hours.

Following 4 hour incubation, a luciferase assay was performed using the Luciferase Assay System (Promega, E1501) according to the manufacturer's instructions. Briefly, medium was aspirated and 25 μl 1× is Buffer (Promega, E153A) was added to each well. Plates were incubated for 20-30 minutes at RT to equilibrate. Luciferase activity was measured using a microplate luminometer (Berthold Technologies), 40 μl substrate injection, 1 second integration time. Data was analyzed using analytical software (Spotfire) and IC₅₀ values were calculated for each candidate and control.

The act of VEGF-A₁₆₅ binding to its receptor, VEGF-R2 (KDR/Flk-1), induces a signaling cascade that activates STAT (signal transducer and activator of transcription) and/or SRE (serum-response element) which drives transcription of the luciferase reporter gene. A decrease in luciferase activity indicates that this VEGF-A-mediated signaling is being neutralized.

Results: scFvs listed in Table 6 below were screened in the luciferase assay for neutralizing VEGF-induced activity. Significant inhibition was demonstrated with a number of scFvs screened (reported as IC50 values in Table 6). IC50 values are indicated as nM concentration of scFv needed to neutralize VEGF-activity by 50%. Bevacizumab (Avastin™), Lucentis™, and Avastin™ Fab (generated in-house) were used as controls for activity.

TABLE 6
Activity in Cell-based Luciferase Assay scFvs and controls

| | IC50 (nM) |
|---|---|
| Avastin ™ | 0.1-0.4 |
| Lucentis ™ | 0.1-0.5 |
| Avastin ™ Fab | 2.9-6.45 |
| c1094.1_1 | 0.95 |
| c870.1_1 | 0.16-0.3 |
| c1039.1_1 | 0.07 |

### Example 8

### Proliferation Assay to Determine Neutralizing Activity of VEGFA scFvs on Human VEGF-A-Stimulated HUVEC Cells

To screen for a neutralizing VEGF-A scFv that had a moderate affinity for VEGF-A, a 3H-thymidine assay was run. Recombinant human VEGF-A165 was used as a positive control at 2.6 nM. DMEM-F12 (1:1) media with 1× insulin-transferrin-selenium (serum-free media, SFM; Invitrogen, Carlsbad, Calif.) was used as a negative control. Human VEGF-A scFv was serially diluted in SFM at 500 nM, 50 nM, 5 nM, 0.5 nM, 0.05 nM, 0.005 nM, and 0.0005 nM. Human umbilical vein endothelial cells (HUVEC) were plated into 96-well flat-bottom plates in a volume of 100 μL at a density of 900-1000 cells per well. The HUVEC cells were plated for 2 days in complete EGM-2 MV media (Lonza, Walkersville, Md.) at 37° C., 5% CO₂. The cells were serum-starved with SFM for 24 h, stimulated for 24 h with 2.6 nM with or without the serially diluted VEGF-A scFv, and pulsed for 24 h with 1 μCi per well of 3H-thymidine, which is incorporated into proliferating cells (all at 37° C., 5% CO₂). The cells were harvested and counted using Topcount instrument (Hewlett Packard).

Results: A large number of scFvs screened in the assay showed potent neutralization of human VEGF-induced HUVEC proliferation as seen by low nM IC50 values as shown in Table 7.

TABLE 7
Anti-VEGF-A scFv Neutralizing Activity in HUVEC Proliferation Assay

| scFvs and controls | IC50 (nM) |
|---|---|
| Avastin ™ | 0.3-0.6 |
| Lucentis ™ | 0.3-0.8 |
| Avastin ™ Fab | 14-17 |
| c1094.1_1 | 1.00 |
| c870.1_1 | 0.8-2 |
| c1039.1_1 | 0.7 |
| c870e6 | 1.6-4 |

### Example 9

### Epitope Binning of VEGF-A Antagonists

Epitope binning experiments were performed to determine which VEGF-A antagonists are capable of binding simultaneously to human VEGF-A. VEGF-A antagonists that compete for the same, or an overlapping, binding site (epitope) on the antigen are not able to bind simultaneously and are functionally grouped into a single family or “epitope bin.” VEGF-A antagonists that do not compete for the same binding site on the antigen are able to bind simultaneously and are grouped into separate families or epitope bins. Experiments were performed using a Biacore T100™ instrument. Biacore is only one of a variety of assay formats that are routinely used to assign panels of antibody fragments and monoclonal antibodies to epitope bins. Many references (e.g., The Epitope Mapping Protocols, Methods in Molecular Biology, Volume 6,6 Glenn E. Morris ed.) describe alternative methods that can be used to “bin” the antibody fragments and which would be expected to provide comparable data regarding the binding characteristics of the VEGF-A antagonists to human VEGF-A. Epitope binning experiments were performed with soluble, native human VEGF-A as the antigen.

Materials and Methods: Two separate epitope binning experiments were performed on a BIACORE T100™ system (GE Healthcare, Piscataway, N.J.). In both experiments, the primary VEGF-A antagonists were covalently immobilized on a CM5 sensor chip using amine coupling chemistry (EDC:NHS) to a density of approximately 800-1000 RU. After the immobilization procedure, remaining active sites on the flow cell were blocked with ethanolamine. Non-specifically bound protein was removed by washing with 50 mM NaOH. The reference cell was also activated and then blocked with ethanolamine without the VEGF-A antagonist.

In the first set of experiments, secondary VEGF-A antagonists and the VEGF-A antigen were diluted to 100 nM. VEGF-A antigen was injected and allowed to specifically bind to a VEGF-A antagonist immobilized on the sensor chip. VEGF-A is a dimer, therefore there are two potential binding sites for every VEGF-A antagonist. To ensure all binding sites were occupied, the primary VEGF-A antagonist that was previously immobilized was injected over VEGF-A. Following this step, secondary VEGF-A antagonist was injected to observe simultaneous binding to VEGF-A.

In a second set of binning experiments, primary VEGF-A antagonists were again covalently immobilized to separate flow cells of a BIACORE CM5 sensor chip. In this experiment however, 10 nM VEGF-A antigen was premixed with 1 mM of the secondary VEGF-A antagonists, then injected over the immobilized primary VEGF-A antagonist in a competition format. All binding experiments were performed at 25° C. in a buffer of 10 mM HEPES, 150 mM NaCl, 3 mM EDTA 0.05% Surfactant P20, 1 mg/ml bovine serum albumin, pH 7.4. Buffer injections were also performed to allow for subtraction of instrument noise and drift. Between cycles, capture surface was regenerated after each injection cycle via 60 second injection of 10 mM Glycine, pH 1.5 at 50 ul/min. This removed the bound VEGF-A from the surface. Data was compiled using Biacore T100™ Evaluation software (version 1.1.1).

Both sets of experimental results were interpreted as follows. If the secondary VEGF-A antagonist was not capable of binding to VEGF-A antigen simultaneously with the primary antagonist, it was functionally grouped into a single family or epitope bin. However, if the secondary VEGF-A antagonist was capable of binding the antigen simultaneously with the primary antagonist by showing an increase in mass on the surface of the chip it was grouped into a separate family or epitope bin. Each VEGF-A antagonist was tested against itself as a negative control to establish the level of the background (no-binding) signal.

Results: The purified VEGF-A antagonists were assigned into epitope bins using the binding data from the two set of experiments described above. The signal (RU, response units) reported by the BIACORE™ is directly correlated to the mass on the sensor chip surface. Once the level of background signal (RU) associated with the negative controls was established (the same VEGF-A antagonist used as both the primary and secondary antagonists), the binning results were reported as either positive or negative binding. Positive binding indicates that two different VEGF-A antagonists are capable of binding the antigen simultaneously. Negative binding indicates that two different VEGF-A antagonists are not capable of binding the antigen simultaneously.

The differential between positive and negative response values in these experiments was used to assign the VEGF-A antagonists into three families or epitope bins (see Table 8). The first epitope bin is represented by VEGF-A antagonist produced by clone c636. A second epitope bin is represented by VEGF-A antagonists c868, c1039, and c1081. Of note, when c636 was the first to interact with VEGF-A, both c868 and c1039 showed simultaneous binding. When either c868 or c1039 interacted with VEGF-A first, c636 did not show any binding, therefore c868 and c1039 are overlapping the c636 epitope. In addition, VEGF-A antagonist c870 overlapped bin #1 and bin #2. A third epitope bin is represented by VEGF-A antagonist c820 and the positive control VEGF-A antibody (mouse anti VEGF-A monoclonal antibody, R&D Systems). Both of these VEGF-A antagonists showed simultaneous binding in the presence of all the other VEGF-A antagonists. All of the antagonists tested in the binning experiments were shown to neutralize VEGF-A mitogenic activity to some degree.

TABLE 8
Epitope Bin Assignments for Neutralizing VEGF-A antagonists

| Epitope Bin # | VEGF-Aantagonists |
|---|---|
| Bin#1: | c636 |
| Bin#2: | c868, c1039, c1081 |
| Bin#1/2: | c870 |
| Bin#3: | c820 |

### Example 10

### Measurement of Binding Affinities of Human VEGF-A Antagonists to VEGF-A Via Surface Plasmon Resonance (Biacore)

Monovalent human VEGF-A antagonists produced by clones c870 and c1039 were evaluated for their binding affinities to human VEGF-A using surface plasmon resonance. Affinity Determination Kinetic rate constants and equilibrium dissociation constants were measured for the interaction of VEGF-A antagonists with the VEGF-A via surface plasmon resonance. The association rate constant (kₐ (M₋₁s₋₁)) is a value that reflects the rate of the antigen-antagonist complex formation. The dissociation rate constant (k_{d} (s₋₁)) is a value that reflects the stability of this complex. By dividing the association rate constant by the dissociation rate constant (kₐ/k_{d}) the equilibrium association constant (K_{A} (M₋₁)) is obtained. By dividing the dissociation rate constant by the association rate constant (k_{d}/kₐ) the equilibrium dissociation constant (K_{D} (M)) is obtained. This value describes the binding affinity of the interaction. Interactions with the same K_{D} can have widely variable association and dissociation rate constants. Consequently, measuring both the kₐ and k_{d} helps to more uniquely describe the affinity of the interaction.

Materials and Methods: A series of experiments were completed to measure the binding affinities of purified VEGF-A antagonists produced by clones c870 and c1039. Binding kinetics and affinity studies were performed on a Biacore T100™ system (GE Healthcare, Piscataway, N.J.). Methods were programmed using Biacore T100™ Control Software, v 1.1.1. The VEGF-A antagonists were produced with His₆/Myc epitope tags. Affinity analyses was performed by capturing VEGF-A antagonists using anti-His₆/Myc antibodies immobilized on a CM5 chip. Anti-His₆ and anti-Myc antibodies were mixed in 1:1 molar ratio and covalently immobilized to a CM5 sensor chip using amine coupling chemistry to a density of approximately 7500RU. 10 nM of VEGF-A antagonists were injected on separate flow cells at 10 ul/min for 1 minute, followed with a 1 minute stabilization period. Serial 1:3 dilutions of VEGF-A from 33.3 nM-0.14 nM were injected over this surface and allowed to specifically bind to VEGF-A antagonist captured on the sensor chip. Duplicate injections of each VEGF-A concentration were performed with an association time of 5 minutes and dissociation time of 10 minutes. Kinetic binding studies were performed with a flow rate of 30 μL/min. All binding experiments were performed at 25° C. in a buffer of 10 mM HEPES, 500 mM NaCl, 3 mM EDTA 0.05% Surfactant P20, 0.1 mg/ml bovine serum albumin, pH 7.4. Between cycles, the flow cell was washed with 50 mM H3PO₄ to regenerate the surface. This wash step removed the captured VEGF-A antagonist from the immobilized antibody surface, and allowed for the subsequent binding of the next sample.

Data was compiled using Biacore T100™ Evaluation software (version 1.1.1). Data was processed by subtracting reference flow cell and blank injections. Baseline stability was assessed to ensure that the regeneration step provided a consistent binding surface throughout the sequence of injections. Duplicate injection curves were checked for reproducibility. Since VEGF-A antigen forms dimers, the resulting binding curves were globally fitted to the bivalent analyte interaction model.

Results: Two VEGFA antagonists were characterized for their binding affinity for VEGF-A (results summarized in Table 9). Association rate constants (kₐ (M₋₁s₋₁)) and dissociation rate constants (k_{d} (s₋₁)) were measured for these human VEGF-A antagonists. K_{D} and K_{A} were calculated from the kₐ and k_{d} values. The data fit well to the bivalent analyte model. This model measures two values for both kₐ (kₐ₁ and kₐ₂) and for k_{d} (k_{d1} and k_{d2}). The first set of values (kₐ₁ and k_{d1}) describes the monovalent kinetics of the interaction which are reported in Table 9. The affinity reported for these samples was derived from these values, and is designated K_{D1}. K_{D} and K_{A} were calculated from the kₐ and k_{d} values. All three VEGF-A antagonists showed similar affinity to VEGF-A antigen (K_{D}=0.7-1.0E−9M) and these results were consistent in two independent run.

TABLE 9
Characterization of VEGF-A Antagonist Binding Affinity for VEGF-A

| ID # | ka (M-1s-1) | kd (s-1) | KD (M) | KA (M-1) |
|---|---|---|---|---|
| c868 | 5.E+5 | 6.E−4 | 1.E−9 | 8.E+8 |
| c870 | 2.E+5 | 2.E−4 | 1.E−9 | 1.E+9 |
| c1039 | 3.E+5 | 2.E−4 | 6.E−10 | 2.E+9 |

### Example 11

### Epitope Mapping of Anti-VEGF-A Antibodies

Monoclonal human VEGF-A antibodies produced by clone c870 and c1039 were evaluated for their peptide binding to human VEGF-A using the JPT VEGF-A RepliTope™ slides.

Material and Methods: Each JPT slide consisted of 3 replicates of the following array. Each array consisted of successive, overlapping 13aa fragments of VEGF-A (spots 1-78), followed by successive, overlapping 20aa fragments of VEGF-A (spots 85-115). In addition, control spots of each test antibody and mouse and human IgG flanked top, bottom, and sides of each array. A series of experiments were completed to determine the binding ability of scFvs c870 and c1039 against the synthetic linear peptides of human VEGF-A protein. The anti-human VEGF-A scFvs were labeled with His/Myc epitope tags. A solution of 10-100 μg/ml of the antibodies were applied to the peptide slides. Anti-His and/or anti-Myc antibodies were then applied to the slides. Signals were amplified with the Biotinylated Tyramide according to the method specified by the kit (Renaissance® TSA™ Biotin System, PerkinElmer, #NEL700A). The bound antibodies were visualized using a streptavidin alkaline phosphatase and a DAKO Permanent Red dye.

Data was compiled using a home-made microscope slide scanner consists of a Nikon Eclipse TE2000 U Inverted microscope, an ASI MS-2000 motorized stage, a Photometrics Cascade II 512 camera and a X-cite 120 fluorescent illumination system. The signal intensity was analyzed using the MetaMorph v7.1 imaging software.

Results: The positions and sequences of the binding peptides are shown in Table 10 below. The numbers indicate the percentage signal intensity of the peptide respect to overall signal intensities.

TABLE 10

| Peptide | Peptide | Functional | A2131/ | A2128/ |
|---|---|---|---|---|
| ID | Sequence | unit | c1039 | c870 |
| 7 | HEVVKFMDVYQRS | α1 | 3% | 4% |
| 8 | VVKFMDVYQRSYS | α1 | 6% | 14% |
| 9 | KFMDVYQRSYSHP | α1 | 7% | 17% |
| 10 | MDVYQRSYSHPIE | α1 | 5% | |
| 11 | VYQRSYSHPIETL | α1 | 7% | 6% |
| 18 | DIFQEYPDEIEYI | α2 | 3% | |
| 19 | FQEYPDEIEYIFK | α2 | 5% | |
| 20 | EYPDEIEYIFKPS | β2 | 3% | |
| 23 | EYIFKPSSVPLMR | α2-β2 | 9% | 32% |
| 24 | IFKPSSVPLMRSG | α2-β2 | 7% | 25% |
| 34 | LESVPTEESNITM | β4-β5 | | |
| 36 | PTEESNITMQIMR | β4-β5 | | |
| 37 | EESNITMQIMRIK | β5 | 4% | |
| 38 | SNITMQIMRIKPH | β5 | | |
| 39 | ITMQIMRIKPHQG | β5 | 3% | 1% |
| 41 | IMRIKPHQGQHIG | β5-β6 | | |
| 59 | PSGPSSERRKHLF | post β7 * | 13% | |
| 60 | GPSSERRKHLFVQ | post β7 * | 5% | |
| 75 | ARQLELNERTSRS | post β7 * | 7% | |
| 76 | QLELNERTSRSDK | post β7 * | 4% | |
| 77 | ELNERTSRSDKPR | post β7 * | 4% | |
| 78 | LNERTSRSDKPRR | post β7 * | 4% | |

* The region “post β7” is the heparin binding domain of the VEGF molecule.

All antibodies showed a specific binding site around α2-β2 region. The data indicated that the testing antibodies could be classified into two categories: antibody c870 preferred the c-terminal side of the VEGF, while antibody c1039 had the stronger binding towards the n-terminal side of the protein. Antibody c870 had the fewest binding peptides while antibody c1039 had the most dispersed binding pattern The top two binding sites from each antibody are listed in the table below.

TABLE 11

| Binding | | |
|---|---|---|
| ranks | A2131/c1039 | A2128/c870 |
| 1 | post β7 | α2-β2 |
| 2 | α2-β2 | α1 |

### Example 12

### Testing Cross-Reactivity of VEGF-a-Binding ScFvs and Bispecific Antibodies Against Murine VEGF-A Using the VEGFR2Phosphorylation Assay

To screen candidate molecules (scFvs, Fabs, and bispecifics) for their ability to neutralize the activity of murine VEGF-A, a cell-based luminex assay that measures VEGFR2 (KDR/Flk-1) phosphorylation was performed. Since mVEGF-A₁₆₄ will cross-react to human VEGFR2, a human VEGFR2-based reporter system can be utilized. 293/KDR/KZ136/c22 cells were plated at a density of 20,000 cellsper well in 100 μl complete medium (DMEM, 10% fetal bovine serum (FBS), 1× Sodium Pyruvate, 1× GlutaMax (Invitrogen)) in clear 96-well tissue culture plates and allowed to attach overnight. The following day, complete medium was removed by vacuum aspiration and replaced with 100 μl serumfree medium (DMEM, 1× Sodium Pyruvate, 1× GlutaMax). Cells were incubated overnight.

The following day, candidate VEGF-A neutralizing molecules (scFvs, Fabs) were serially diluted from 200 nM down to 12 pM at 1:5 dilutions along with a non-neutralizer (medium only) in serum-free medium. VEGFR2-Fc was used as a positive control for neutralization. To these, and equal volume of mVEGF-A₁₆₄ (493-MV-005, R&D Systems) was added at 0.54 nM for a final concentration of 0.26 nM VEGF-A and 100 nM to 6 pM neutralizing molecule or positive control. These were incubated for 60 minutes at 37° C.

Following incubation, medium was removed from serum-starved cells by vacuum aspiration and replaced with 100 μl of above complexes. Cells were incubated for 10 minutes at 37° C. Following incubation, medium was removed by vacuum aspiration and cells were gently washed with 100 μl ice-cold phosphate-buffered saline (PBS, Invitrogen). PBS was removed by vacuum aspiration and cells were lysed in 25 μl NP-40 lysis buffer (Invitrogen Cat.#FNN0021) containing 1 mM PMSF (Sigma, P-2714 in DMSO) and 1 Complete Mini tablet per 10 mL (Roche, 11836153001). Lysates were incubated for 20 minutes at 4° C. on a platform shaker and centrifuged at 3000 rpm for 10 min at 4° C. to clear lysates. Lysates were transferred to a fresh 96-well microtiter plate and placed at −20° C. until assay.

For the VEGFR2 phosphorylation luminex assay, the Intracellular Protein Buffer Reagent Kit (Invitrogen LHB0002) and VEGFR2 [pY1059] Antibody Bead Kit (Invitrogen LHO0601) was used according to manufacturer's instructions. Lysates were thawed and mixed 1:5 with 80 μl Assay Diluent. Wells of a luminex vacuum filtration plated were pre-wetted with 200 μl Working Wash Solution. Diluted beads were added at 25 μl per well and washed 2× with 200 μl Working Wash Solution. Following washing, 50 μl of diluted lysate, and 50 μl of diluted detector antibody was added to each well and plates were covered in foil and incubated for 3 hours at room temperature (RT) on a platform shaker at 500 rpm. Following incubation, beads were washed 2× with 200 μl Working Wash Solution and then 100 μl of diluted Anti-Rabbit IgG-RPE was added to each well and plates were covered in foil and incubated for 30 minutes at RT on a platform shaker at 500 rpm. Following incubation, beads were washed 3× with 200 μl Working Wash Solution, and resuspended in 125 μl Working Wash Solution. Beads were resuspended for 30 seconds on a platform shaker at 500 rpm and read in Luminex-100 instrument (BioRad). Data was analyzed using analytical software (Spotfire) and IC₅₀ values were calculated for each candidate and control.

Results: The act of mVEGF-A₁₆₄ binding to human receptor, VEGF-R2 (KDR/Flk-1), induces phosphorylation of the receptor. This luminex-based assay binds total VEGF-R2 to a fluorescently labeled bead conjugated to an anti-VEGFR2 antibody. A secondary antibody detecting phosphorylation at [pY1059] is used to detect how much VEGFR2 has been phosphorylated. As shown in Table 12 below, a number of scFvs that neutralized human VEGF-A activity also inhibited mouse VEGF activity in this assay. Bispecific antibodies that contained these same scFvs also neutralized mouse VEGF-A activity.

TABLE 12
Neutrlaization of Mouse VEGF-A Activity by VEGF-A-specific
scFvs and Bispecific Antibodies

| scFv | IC₅₀ (nM) |
|---|---|
| c870 | 0.16 |
| c1039 | no activity |
| c1094 | 0.55 |

### Example 13

### Proliferation Assay to Determine Neutralizing Activity of scFvs on Mouse VEGFA (VEGF-A₁₆₄)-Stimulated HUVEC Cells

To screen for mouse VEGF-A neutralizing scFvs, a ₃H-thymidine assay was run. Recombinant mouse VEGF-A₁₆₄ was used as a positive control at 2.6 nM. DMEM-F12 (1:1) media with 1× insulin-transferrin-selenium (serum-free media, SFM; Invitrogen, Carlsbad, Calif.) was used as a negative control. scFv molecules were serially diluted in SFM at 500 nM, 50 nM, 5 nM, 0.5 nM, 0.05 nM, 0.005 nM, and 0.0005 nM. Human umbilical vein endothelial cells (HUVEC) were plated into 96-well flat bottom plates in a volume of 100 μL at a density of 900-1000 cells per well. The HUVEC cells were plated for 2 days in complete EGM-2 MV media (Lonza, Walkersville, Md.) at 37° C., 5% CO₂. The cells were serum-starved with SFM for 24 h, stimulated for 24 h with 2.6 nM with or without the serially diluted VEGF-A scFv, and pulsed for 24 h with 1 μCi per well of 3H thymidine, which is incorporated into proliferating cells (all at 37° C., 5% CO₂). The cells were harvested and counted using Topcount instrument (Hewlett Packard).

Results: A large number of scFvs screened in the assay showed potent neutralization of mouse VEGF-induced HUVEC proliferation, as seen by low nM IC₅₀ values shown in the Table 13 below.

TABLE 13
Neutrlaization of Mouse VEGF-A-induced HUVEC proliferation
by VEGF-A-specific scFvs

| scFv | IC₅₀ (nM) |
|---|---|
| c870 | 0.36 |
| c1094 | 2.84 |
| c1039 | no activity |

### Example 14

### Construction of Soluble FGFR3IIIc C-term Fc5 Expression Plasmids to Express the 1^{st}, 2^{nd} and 3rd Extracellular Ig like domains or a Truncated Form Including the 2^{nd} and 3rd Extracellular Ig like Domains

A series of expression constructs containing the first, second and third extracellular Ig like domains of Human FGFR3IIIc or a truncated form with the second and third extracellular Ig like domains of Human FGFR3IIIc were generated. These Human FGFR3IIIc sequence spans were fused with a downstream C-terminal Fc5 sequence. Constructs in this series included the sequence spans mentioned above and a point mutation that yielded a Tryptophan residue at amino acid residue 262 of SEQ ID NO:2 and residue 142 of SEQ ID NO:10. (The 249 position of the mutation is in reference to the native FGFR3IIIc Amino Acid sequence) instead of a Serine at this position. This mutation is noted as S249W. These constructs were generated via PCR and homologous recombination using DNA fragments encoding the FGFR3IIIc domains noted above, Fc5 fragment and the expression vector pZMP31.

To generate the full length soluble Human FGFR3IIIc (S249W) Fc5 construct designated MPET construct #1917 (SEQ ID NOS:1 and 2), three PCR fragments were generated and together introduced into the pZMP31 vector via yeast recombination. The first fragment represented a 5′ overlap with an optimized TPA leader in the pZMP31 vector sequence followed by sequence of Human FGFR3IIIc encoding a S249W point mutation and a 3′ overlap with downstream Human FGFR3IIIc sequence. For this fragment, the PCR amplification reactions used the 5′ oligonucleotides zc62552 ((SEQ ID NO:3) (Forward primer to generate a PCR frag using FGFR3 IIIc as template. FGFR3IIIc starts at E36 of SEQ ID NO:2)). Fragment to be cloned into pZMP31 utilizing the opTPA leader sequence (residues 1-35 of SEQ ID NO:2). The PCR was run with the 3′ oligonucleotides zc62557 (SEQ ID NO:4) (Reverse primer to generate a PCR frag using FGFR3 IIIc as template. Fragment will generate a S249W mutation. To be used with a Forward primer nested at 5′ end of Rec sequence), and utilized clonetrack ID #102551 Human FGFR3IIIc as template.

The second fragment represented a 5′ overlap with upstream Human FGFR3IIIc sequence followed by sequence of Human FGFR3IIIc encoding a S249W point mutation and a 3′ overlap with Fc5 sequence (residues 389-620 of SEQ ID NO:2). For this fragment, the PCR amplification reactions used the 5′ oligonucleotides zc62556 (SEQ ID NO:82) (forward primer to generate a PCR frag using FGFR3 IIIc as template. Fragment will generate a S249W mutation. To be used with a reverse primer nested at 3′ end of sol. Rec sequence). The PCR was run with the 3′ oligonucleotides zc62553 (SEQ ID NO:6): (Reverse primer to generate a PCR frag using FGFR3 IIIc as template. Seq of sol. FGFR3 IIIc to G 375. Fragment will have overlapping Fc5 sequence), and utilized clonetrack ID #102551 Human FGFR3IIIc as template.

The third fragment contained Fc5 sequence and represented a 5′ overlap with Human FGFR3IIIc and a 3′ overlap the pZMP31 vector sequence. For this fragment, the PCR amplification reactions used the 5′ oligonucleotides zc62554 (SEQ ID NO:7). (Forward primer to generate a PCR frag using Fc5 as template. Fragment will have overlapping Seq of sol. FGFR3 IIIc to G 375). The PCR was run with the 3′ oligonucleotides zc62555 (SEQ ID NO:8). (Reverse primer to generate a PCR frag using Fc5 as template. Fragment will have overlapping seq of pZMP31), and utilized MPET construct #1699, IL17REFc5 as template.

The PCR amplification reaction conditions to generate the three fragments noted above were as follows: 1 cycle, 95° C., 5 minutes; 25 cycles, 95° C., 30 seconds, followed by 55° C., 30 seconds, followed by 68° C., 1 minute 30 seconds; 1 cycle, 72° C., 7 minutes. The PCR reaction mixtures were run on a 1% agarose gel and the DNA fragments corresponding to the expected size is were extracted from the gel using a QIAquick™ Gel Extraction Kit (Qiagen, Cat. No. 28704).

The plasmid pZMP31 is a mammalian expression vector containing an expression cassette having the chimeric CMV enhancer/MPSV promoter, Fse1, Nar1 and a BglII site for linearization prior to yeast recombination, an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae.

The plasmid pZMP31 was digested with BglII prior to recombination in yeast with the following gel extracted PCR fragments mentioned above. Fifty μl of competent yeast (S. cerevisiae) cells were combined with 3 μl of each PCR fragment insert DNA and apx. 50 ng of BglII digested pZMP31 vector. The mix was transferred to a 0.2 cm electroporation cuvette. The yeast/DNA mixture was electropulsed using power supply (BioRad Laboratories, Hercules, Calif.) settings of 0.75 kV (5 kV/cm), ∞ ohms, and 25 μF. Three hundred μl of 1.2 M sorbitol was added to the cuvette, and the yeast was plated in 75 μl and 200 μl aliquots onto two URA-DS plates and incubated at 30° C. After about 72 hours, the Ura⁺ yeast transformants from a single plate were resuspended in 100 ul of yeast lysis buffer (In house: 0.1M NaCL, 0.0062M Tris HCL, 0.0038M Tris Base, 0.001M EDTA, 2% (v/v) polysorbate 20, 1% (w/v) SDS) and 100 ul of Qiagen MiniPrep kit buffer P1 containing 10 U Zymolyase/100 ul. This mixture was then incubated at 37 Deg C. for apx 15 min and the rest of the Qiagen miniprep kit protocol was followed according to manufactures instructions.

Transformation of electrocompetent E. coli host cells (DH12S) was performed using 4 μl of the yeast DNA preparation and 50 μl of E. coli cells. The cells were electropulsed at 1.75 kV, 25 μF, and 400 ohms. Following electroporation, 0.5 ml LB was added and then the cells were plated in 10 μl and 30 μl aliquots on two LB AMP plates (LB broth (Lennox), 1.8% Bacto™ Agar (Difco), 100 mg/L Ampicillin).

The inserts DNA clones were subjected to sequence analysis. One clone containing the correct sequence is selected. Large-scale plasmid DNA is isolated using a commercially available kit (QIAGEN Plasmid Mega Kit, Qiagen, Valencia, Calif.) according to manufacturer's instructions.

The same process was used to prepare the truncated soluble Human FGFR3IIIc (S249W) Fc5 construct, designated MPET construct #1920 (SEQ ID NOS:9 and 10). The first fragment represented a 5′ overlap with an optimized TPA leader in the pZMP31 vector sequence followed by sequence of Human FGFR3IIIc encoding a S249W point mutation and a 3′ overlap with downstream Human FGFR3IIIc sequence. For this fragment, the PCR amplification reactions used the 5′ oligonucleotides zc62560 ((SEQ ID NO:11) (Forward primer to generate a PCR frag using FGFR3 IIIc as template. Fragment will generate a Ig D2 D3 form. Sequence of the truncated FGFR3 IIIc starts at D156 of SEQ ID NO:2, upstream of Ig D2. Fragment to be cloned into pZMP31 utilizing the opTPA leader seq.)). The PCR was run with the 3′ oligonucleotides zc62557 (SEQ ID NO:4) (Reverse primer to generate a PCR frag using FGFR3 IIIc as template. Fragment will generate a S249W mutation. To be used with a Forward primer nested at 5′ end of Rec sequence)), and utilized clonetrack ID #102551 Human FGFR3IIIc as template. This fragment was generated utilizing the same PCR thermocycles noted earlier and introduced into the BglII digested pzMP31 vector along with the second and third fragments noted above to generate a truncated soluble Human FGFR3IIIc (S249W) Fc5 construct.

Mega prep DNA was prepared for each plasmid using a Qiagen Plasmid Mega Kit (Qiagen, Valencia, Calif.). For the full length soluble Human FGFR3IIIc (S249W) Fc5, and the Truncated soluble Human FGFR3IIIc (S249W) Fc5 constructs, designated MPET construct #1917 (SEQ ID NOS: 1 and 2) and #1920 (SEQ ID NOS:9 and 10) respectively, 200 μg of each of the expression constructs mega prep plasmid were digested with apx 240 units of BstB1 restriction enzyme at 37° C. for 2 hours, washed with phenol/chloroform/isoamyl alcohol, followed by a wash with chloroform/isoamyl, then precipitated overnight with ethanol, and centrifuged in a 1.5 mL microfuge tube. The supernatants were decanted and the pellets were washed with 1 mL of 70% ethanol and allowed to incubate for 5 minutes at room temperature. The tubes were spun in a microfuge for 10 minutes at 14,000 RPM and the supernatants were decanted off the pellets. In the sterile environment on the tissue culture hood, the pellets were allowed to dry in the open air for apx. 5 min, then resuspended in 0.4 mls of 37° C., pre-warmed CHO cell tissue culture medium and allowed to incubate at 37° C. for 10 minutes. While the DNA pellets were being solubilized, for each vector to be electroporated, approximately 9×10⁶ CHO cells were pelleted and resuspended in 0.4 mls of CHO cell tissue culture medium and combined with the resuspended plasmid DNA for a final volume of 800 ul. The DNA/cell mixtures were placed in a 0.4 cm gap cuvette and electroporated using the following parameters; 950 μF, high capacitance, at 300 V. For each plasmid electroporation set, the contents of the cuvettes were then removed, pooled, and diluted to 25 mLs with CHO cell tissue culture medium and placed in a 125 mL shake flask. The flask was placed in an incubator on a shaker at 37° C., 5% CO₂ with shaking at 120 RPM.

The CHO cells were subjected to nutrient selection and amplification to 500 nM Methotrexate (MTX). The selected CHO lines were designated MECL 1334 (solFGFR3IIIc(23_{—}375)(S249W)Fc5) and MECL 1337 (solFGFR3IIIc(143_{—}375)(S249W)Fc5).

To test for expression, cultures were set up using passage 3 post-electroporation pools. Cells were centrifuged and resuspended in fresh media without selection in a 50 ml volume at 0.5e6 c/ml and allowed to proceed as previously described for 96 hrs. Protein expression was confirmed by Western blot.

Large scale spinner flasks were initiated to generate protein for purification. Two hundred ml seed cultures were started on passage 6, post-transfected CHO cell pools of MECL 1334 and MECL 1337 using ZM2 medium (SAFC Biosciences Ex-CELL catalog #68041) with the addition of 5 mM L-glutamine (from 200 mM L-glutamine, Gibco catalog #25030-081), 1 mM sodium pyruvate (from 100 mM Sodium Pyruvate, Gibco catalog #11360-070) and 500 nM methotrexate. The flasks were cultured @ 37 deg. C., 120 rpm and 6% CO2. After 6 days, each CHO pool flask was seeded into a 3 L spinner flask to attain a 1 L working volume at 0.5e6c/ml using ZM2 medium (SAFC Biosciences Ex-CELL catalog #68041) with the addition of 5 mM L-glutamine (from 200 mM L-glutamine, Gibco catalog #25030-081), 1 mM sodium pyruvate (from 100 mM Sodium Pyruvate, Gibco catalog #11360-070) without selection. The spinner flasks were cultured at 37° C., 95 rpm and 6% CO2. After approximately 24 hrs post seed, an additional 0.5 L of media was added to each spinner to achieve a final volume of 1.5 L and the cultures were allowed to continue. After apx. Eight days post seed, the conditioned medium was harvested, 0.2 μM filtered and submitted for protein purification.

Similar methods were used to generate additional constructs to the FGFR3 Fc5 field including both soluble Full length and truncated versions without point mutations or with a mutation in the sequence such that the Proline residue in position 263 of SEQ ID NO:15 and position 143 of SEQ ID NO:22 is changed to a Arginine noted as P250R (The 250 position of the mutation is in reference to the native FGFR3IIIc Amino Acid sequence). The oligonucleotides and resulting constructs are shown below with the corresponding sequence identifiers.

TABLE 14
pZMP31solFGFR3IIIc(23_375)(S249W)Fc5,
Construct #1917 (SEQ ID NOS: 1 and 2)

| | | |
|---|---|---|
| zc62552 | CAGGAAATCCATGCCGAGTTGAGACGCTTCC | SEQ ID |
| | GTAGAGAGTCCTTGGGGACGGAGCAGCGC | NO: 3 |
| zc62557 | CGCCTGCAGGATGGGCCGGTGCGGCCAGCGC | SEQ ID |
| | TCCAGCACGTCCAGCGTGT | NO: 4 |
| zc62556 | TACACGCTGGACGTGCTGGAGCGCTGGCCGCA | SEQ ID |
| | CCGGCCCATCCTGCAGGCG | NO: 5 |
| zc62553 | TGGGCATGTGTGAGTTTTGTCTGAAGATTTGGG | SEQ ID |
| | CTCGCCTGCATACACACTGCCCGCCTC | NO: 6 |
| zc62554 | GTGGAGGCTGACGAGGCGGGCAGTGTGTATGCA | SEQ ID |
| | GGCGAGCCCAAATCTTCAGACAAAACT | NO: 7 |
| zc62555 | CAACCCCAGAGCTGTTTTAAggcgcgccTCTAG | SEQ ID |
| | ATTATTTACCCGGAGACAGGGAGAGGCT | NO: 8 |

TABLE 15
pZMP31solFGFR3IIIc(143_375)(S249W)Fc5, Construct
#1920 (SEQ ID NOS: 9 and 10)

| | | |
|---|---|---|
| zc62560 | CAGGAAATCCATGCCGAGTTGAGACGCT | SEQ ID |
| | TCCGTAGAGACACAGGTGTGGACACAGGGGCC | NO: 11 |
| zc62557 | CGCCTGCAGGATGGGCCGGTGCGGCCAG | SEQ ID |
| | CGCTCCAGCACGTCCAGCGTGT | NO: 4 |
| zc62556 | TACACGCTGGACGTGCTGGAGCGCTGGCC | SEQ ID |
| | GCACCGGCCCATCCTGCAGGCG | NO: 5 |
| zc62553 | TGGGCATGTGTGAGTTTTGTCTGAAGATTT | SEQ ID |
| | GGGCTCGCCTGCATACACACTGCCCGCCTC | NO: 6 |
| zc62554 | GTGGAGGCTGACGAGGCGGGCAGTGTGTAT | SEQ ID |
| | GCAGGCGAGCCCAAATCTTCAGACAAAACT | NO: 7 |
| zc62555 | CAACCCCAGAGCTGTTTTAAggcgcgccTCTAG | SEQ ID |
| | ATTATTTACCCGGAGACAGGGAGAGGCT | NO: 8 |

TABLE 16
pZMP31solFGFR3IIIc(23_375)Fc5, Construct #1916
(SEQ ID NOS: 12 and 13)

| | | |
|---|---|---|
| zc62552 | CAGGAAATCCATGCCGAGTTGAGACGCT | SEQ ID |
| | TCCGTAGAGAGTCCTTGGGGACGGAGCAGCGC | NO: 3 |
| zc62553 | TGGGCATGTGTGAGTTTTGTCTGAAGATTT | SEQ ID |
| | GGGCTCGCCTGCATACACACTGCCCGCCTC | NO: 6 |
| zc62554 | GTGGAGGCTGACGAGGCGGGCAGTGTGTAT | SEQ ID |
| | GCAGGCGAGCCCAAATCTTCAGACAAAACT | NO: 7 |
| zc62555 | CAACCCCAGAGCTGTTTTAAggcgcgccTCTAG | SEQ ID |
| | ATTATTTACCCGGAGACAGGGAGAGGCT | NO: 8 |

TABLE 17
pZMP31solFGFR3IIIc(23_375)(P250R)Fc5, Construct
#1918 (SEQ ID NOS: 14 and 15)

| | | |
|---|---|---|
| zc62552 | CAGGAAATCCATGCCGAGTTGAGACGCT | SEQ ID |
| | TCCGTAGAGAGTCCTTGGGGACGGAGCAGCGC | NO: 3 |
| zc62553 | TGGGCATGTGTGAGTTTTGTCTGAAGATTT | SEQ ID |
| | GGGCTCGCCTGCATACACACTGCCCGCCTC | NO: 6 |
| zc62554 | GTGGAGGCTGACGAGGCGGGCAGTGTGTAT | SEQ ID |
| | GCAGGCGAGCCCAAATCTTCAGACAAAACT | NO: 7 |
| zc62555 | CAACCCCAGAGCTGTTTTAAggcgcgccTCTAG | SEQ ID |
| | ATTATTTACCCGGAGACAGGGAGAGGCT | NO: 8 |
| zc62558 | CCCCGCCTGCAGGATGGGCCGGTGCCGGGAG | SEQ ID |
| | CGCTCCAGCACGTCCAGCGT | NO: 16 |
| zc62559 | ACGCTGGACGTGCTGGAGCGCTCCCGGCACCG | SEQ ID |
| | GCCCATCCTGCAGGCGGGG | NO: 17 |

TABLE 18
pZMP31solFGFR3IIIc(143_375)Fc5, Construct #1919
(SEQ ID NOS: 18 and 19)

| | | |
|---|---|---|
| zc62553 | TGGGCATGTGTGAGTTTTGTCTGAAGATTT | SEQ ID |
| | GGGCTCGCCTGCATACACACTGCCCGCCTC | NO: 6 |
| zc62554 | GTGGAGGCTGACGAGGCGGGCAGTGTGTAT | SEQ ID |
| | GCAGGCGAGCCCAAATCTTCAGACAAAACT | NO: 7 |
| zc62555 | CAACCCCAGAGCTGTTTTAAggcgcgccTCTAG | SEQ ID |
| | ATTATTTACCCGGAGACAGGGAGAGGCT | NO: 8 |
| zc62560 | CAGGAAATCCATGCCGAGTTGAGACGCTTCCG | SEQ ID |
| | TAGAGACACAGGTGTGGACACAGGGGCC | NO: 20 |

TABLE 19
pZMP31solFGFR3IIIc(143_375)(P250R)Fc5, Construct
#1921 (SEQ ID NOS: 21 and 22)

| | | |
|---|---|---|
| zc62553 | TGGGCATGTGTGAGTTTTGTCTGAAGATTT | SEQ ID |
| | GGGCTCGCCTGCATACACACTGCCCGCCTC | NO: 6 |
| zc62554 | GTGGAGGCTGACGAGGCGGGCAGTGTGTAT | SEQ ID |
| | GCAGGCGAGCCCAAATCTTCAGACAAAACT | NO: 7 |
| zc62555 | CAACCCCAGAGCTGTTTTAAggcgcgccTCTAG | SEQ ID |
| | ATTATTTACCCGGAGACAGGGAGAGGCT | NO: 8 |
| zc62558 | CCCCGCCTGCAGGATGGGCCGGTGCCGGGAG | SEQ ID |
| | CGCTCCAGCACGTCCAGCGT | NO: 16 |
| zc62559 | ACGCTGGACGTGCTGGAGCGCTCCCGGCACCG | SEQ ID |
| | GCCCATCCTGCAGGCGGGG | NO: 17 |
| zc62560 | CAGGAAATCCATGCCGAGTTGAGACGCTTCCGTA | SEQ ID |
| | GAGACACAGGTGTGGACACAGGGGCC | NO: 20 |

Similar methods were used to generate a field of constructs for soluble forms of FGFR2alphaIIIc Fc5 including both soluble Full length and truncated versions without point mutations or with mutations in the sequence such that the Serine residue in position 266 of SEQ ID NO:29 and position 143 of SEQ ID NO:40 is changed to a Tryptophan noted as S252W or Proline residue in position 267 of SEQ ID NO:33 and position 144 of SEQ ID NO:42 is changed to a Arginine noted as P253R (The positions of the mutations is in reference to the native FGFR2alphaIIIc Amino Acid sequence).

TABLE 20
pZMP31solFGFR2alphaIIIc(22_377)Fc5, Construct
#1945 (SEQ ID NOS: 23 and 24)

| | | |
|---|---|---|
| zc62932 | CAGGAAATCCATGCCGAGTTGAGACGCTTC | SEQ ID |
| | CGTAGACGGCCCTCCTTCAGTTTAGTTGAG | NO: 25 |
| zc62933 | TGGGCATGTGTGAGTTTTGTCTGAAGATTTGG | SEQ ID |
| | GCTCCTCCAGGTAGTCTGGGGAAGCTGT | NO: 26 |
| zc62934 | GAAAAGGAGATTACAGCTTCCCCAGACTACCTG | SEQ ID |
| | GAGGAGCCCAAATCTTCAGACAAAAC | NO: 27 |
| zc62555 | CAACCCCAGAGCTGTTTTAAggcgcgccTCTAG | SEQ ID |
| | ATTATTTACCCGGAGACAGGGAGAGGCT | NO: 8 |

TABLE 21
pZMP31solFGFR2alphaIIIc(22_377)(S252W)Fc5,
Construct #1946 (SEQ ID NOS: 28 and 29)

| | | |
|---|---|---|
| zc62932 | CAGGAAATCCATGCCGAGTTGAGACGCTTC | SEQ ID |
| | CGTAGACGGCCCTCCTTCAGTTTAGTTGAG | NO: 25 |
| zc62933 | TGGGCATGTGTGAGTTTTGTCTGAAGATTTGG | SEQ ID |
| | GCTCCTCCAGGTAGTCTGGGGAAGCTGT | NO: 26 |
| zc62934 | GAAAAGGAGATTACAGCTTCCCCAGACTACCTG | SEQ ID |
| | GAGGAGCCCAAATCTTCAGACAAAAC | NO: 27 |
| zc62555 | CAACCCCAGAGCTGTTTTAAggcgcgccTCTAG | SEQ ID |
| | ATTATTTACCCGGAGACAGGGAGAGGCT | NO: 8 |
| zc62937 | GGCTTGGAGGATGGGCCGGTGAGGCCATCGC | SEQ ID |
| | TCCACAACATCCAGGTGGTA | NO: 30 |
| zc62936 | TACCACCTGGATGTTGTGGAGCGATGGCCTCA | SEQ ID |
| | CCGGCCCATCCTCCAAGCC | NO: 31 |

TABLE 22
pZMP31solFGFR2alphaIIIc(22_377)(P253R)Fc5,
Construct #1947 (SEQ ID NOS: 32 and 33)

| | | |
|---|---|---|
| zc62932 | CAGGAAATCCATGCCGAGTTGAGACGCTTC | SEQ ID |
| | CGTAGACGGCCCTCCTTCAGTTTAGTTGAG | NO: 25 |
| zc62933 | TGGGCATGTGTGAGTTTTGTCTGAAGATTTGG | SEQ ID |
| | GCTCCTCCAGGTAGTCTGGGGAAGCTGT | NO: 26 |
| zc62934 | GAAAAGGAGATTACAGCTTCCCCAGACTACCTG | SEQ ID |
| | GAGGAGCCCAAATCTTCAGACAAAAC | NO: 27 |
| zc62939 | TCCGGCTTGGAGGATGGGCCGGTGACGCGATCG | SEQ ID |
| | CTCCACAACATCCAGGTG | NO: 34 |
| zc62938 | CACCTGGATGTTGTGGAGCGATCGCGTCACCGG | SEQ ID |
| | CCCATCCTCCAAGCCGGA | NO: 35 |
| zc62555 | CAACCCCAGAGCTGTTTTAAggcgcgccTCTAG | SEQ ID |
| | ATTATTTACCCGGAGACAGGGAGAGGCT | NO: 8 |

TABLE 23
pZMP31solFGFR2alphaIIIc(145_377)Fc5, Construct
#1948 (SEQ ID NOS: 36 and 37)

| | | |
|---|---|---|
| zc62935 | CAGGAAATCCATGCCGAGTTGAGACGCTTCC | SEQ ID |
| | GTAGAAGTGAGAACAGTAACAACAAGAGA | NO: 38 |
| zc62933 | TGGGCATGTGTGAGTTTTGTCTGAAGATTTGG | SEQ ID |
| | GCTCCTCCAGGTAGTCTGGGGAAGCTGT | NO: 26 |
| zc62934 | GAAAAGGAGATTACAGCTTCCCCAGACTACCTG | SEQ ID |
| | GAGGAGCCCAAATCTTCAGACAAAAC | NO: 27 |
| zc62555 | CAACCCCAGAGCTGTTTTAAggcgcgccTCTAG | SEQ ID |
| | ATTATTTACCCGGAGACAGGGAGAGGCT | NO: 8 |

TABLE 24
pZMP31solFGFR2alphaIIIc(145_377)(S252W)Fc5,
Construct #1949 (SEQ ID NOS: 39 and 40

| | | |
|---|---|---|
| zc62935 | CAGGAAATCCATGCCGAGTTGAGACGCTTCCGTA | SEQ ID |
| | GAAGTGAGAACAGTAACAACAAGAGA | NO: 38 |
| zc62937 | GGCTTGGAGGATGGGCCGGTGAGGCCATCGC | SEQ ID |
| | TCCACAACATCCAGGTGGTA | NO: 30 |
| zc62936 | TACCACCTGGATGTTGTGGAGCGATGGCCTCA | SEQ ID |
| | CCGGCCCATCCTCCAAGCC | NO: 31 |
| zc62934 | GAAAAGGAGATTACAGCTTCCCCAGACTACCTG | SEQ ID |
| | GAGGAGCCCAAATCTTCAGACAAAAC | NO: 27 |
| zc62555 | CAACCCCAGAGCTGTTTTAAggcgcgccTCTAG | SEQ ID |
| | ATTATTTACCCGGAGACAGGGAGAGGCT | NO: 8 |

TABLE 25
pZMP31solFGFR2alphaIIIc(145_377)(P253R)Fc5,
Construct #1950 (SEQ ID NOS: 41 and 42)

| | | |
|---|---|---|
| zc62935 | CAGGAAATCCATGCCGAGTTGAGACGCTTCCG | SEQ ID |
| | TAGAAGTGAGAACAGTAACAACAAGAGA | NO: 38 |
| zc62933 | TGGGCATGTGTGAGTTTTGTCTGAAGATTTGG | SEQ ID |
| | GCTCCTCCAGGTAGTCTGGGGAAGCTGT | NO: 26 |
| zc62934 | GAAAAGGAGATTACAGCTTCCCCAGACTACCTG | SEQ ID |
| | GAGGAGCCCAAATCTTCAGACAAAAC | NO: 27 |
| zc62939 | TCCGGCTTGGAGGATGGGCCGGTGACGCGATCGC | SEQ ID |
| | TCCACAACATCCAGGTG | NO: 34 |
| zc62938 | CACCTGGATGTTGTGGAGCGATCGCGTCACCGGCCC | SEQ ID |
| | ATCCTCCAAGCCGGA | NO: 35 |
| zc62555 | CAACCCCAGAGCTGTTTTAAggcgcgccTCTAG | SEQ ID |
| | ATTATTTACCCGGAGACAGGGAGAGGCT | NO: 8 |

### Example 15

### Construction of Multi-specific, Soluble FGFR3IIIc Fc5 VEGFA scFv Expression Plasmids

A series of expression constructs containing the first, second and third extracellular Ig like domains of Human FGFR3_{III}c or a truncated form with the second and third extracellular Ig like domains of Human FGFR3_{III}c were generated. These Human FGFR3_{III}c sequence spans were fused with a downstream C-terminal Fc5, a linker and downstream, c-terminal scFv sequences specific for binding to VEGF-A. Constructs in this series included the Human FGFR3_{III}c sequence spans mentioned above and a point mutation that yielded a Tryptophan residue at amino acid position 162 of SEQ ID NO:64 and 142 of SEQ ID NO:62 instead of a Serine at this position. This mutation is noted as S249W. (The position of the mutation is in reference to the native FGFR3_{III}c Amino Acid sequence). These constructs were generated via PCR and homologous recombination using DNA fragments encoding the FGFR3IIIc domains with Fc5 sequence and the expression vector pZMP31 which contained the VEGF-A scFv sequences 870e6 and 1094.1.

To generate the full length soluble Human FGFR3_{III}c (S249W) Fc5 c870e6 and full length soluble Human FGFR3_{III}c (S249W) Fc5 c1094.1 constructs, a PCR fragment was generated and introduced into the pZMP31 based vectors which contained the linker and downstream VEGFA scFv sequences 870e6 or 1094.1 (designated MVC 709-SEQ ID NO:43 and 44; and MVC 710-SEQ ID NO:45 and 46) via yeast recombination. The PCR fragment represented a 5′ overlap with an optimized TPA leader in the pZMP31 based vector sequences followed by sequence of Human FGFR3_{III}c encoding a S249W point mutation, Fc5 sequence and a 3′ overlap with a linker sequence. For this fragment, the PCR amplification reactions used the 5′ oligonucleotides zc62552 (SEQ ID NO: 3) (Forward primer to generate a PCR frag using FGFR3_{III}c as template. Seq of FGFR3_{III}c starts at E23 upstream of Ig D1. Fragment to be cloned into pZMP31 utilizing the opTPA leader seq). The PCR was run with the 3′ oligonucleotides zc60566 (SEQ ID NO:82) and utilized MPET construct #1917 (SEQ ID NO:1) as template.

The PCR amplification reaction conditions to generate the three fragments noted above were as follows: 1 cycle, 95° C., 5 minutes; 25 cycles, 95° C., 30 seconds, followed by 55° C., 30 seconds, followed by 68° C., 2 minutes; 1 cycle, 72° C., 7 minutes. The PCR reaction mixtures were run on a 1% agarose gel and the DNA fragments corresponding to the expected size is were extracted from the gel using a QIAquick™ Gel Extraction Kit (Qiagen, Cat. No. 28704).

The plasmids MVC 709 and MVC 710 are pZMP31 based mammalian expression vectors which contain Murine Fc2, a linker and downstream sequence of the 870e6 (SEQ ID NO:43) or 1094.1 scFv (SEQ ID NO:45), VEGF-A binding sequences. These vectors contain an expression cassette having the chimeric CMV enhancer/MPSV promoter, Fse1, Nar1 and a BglII site for linearization prior to yeast recombination, an E. coli origin of replication; a mammalian selectable marker expression unit comprising an SV40 promoter, enhancer and origin of replication, a DHFR gene, and the SV40 terminator; and URA3 and CEN-ARS sequences required for selection and replication in S. cerevisiae.

The plasmids MVC 709 and MVC 710 were digested with BglII restriction enzyme prior to recombination in yeast with the following gel extracted PCR fragments mentioned above. Sixty μl of competent yeast (S. cerevisiae) cells were combined with 5 μl of each PCR fragment insert DNA and apx. 50 ng of BglII digested MVC 709 and MVC 710 vectors. The mix was transferred to a 0.2 cm electroporation cuvette. The yeast/DNA mixture was electropulsed using power supply (BioRad Laboratories, Hercules, Calif.) settings of 0.75 kV (5 kV/cm), ∞ ohms, and 25 μF. Four hundred μl of 1.2 M sorbitol was added to the cuvette, and the yeast was plated in 75 μl and 200 μl aliquots onto two URA-DS plates and incubated at 30° C. After about 72 hours, the Ura⁺ yeast transformants from a single plate were resuspended in 100 ul of yeast lysis buffer (In house: 0.1M NaCL, 0.0062M Tris HCL, 0.0038M Tris Base, 0.001M EDTA, 2% (v/v) polysorbate 20, 1% (w/v) SDS) and 100 ul of Qiagen MiniPrep kit buffer P1 containing 10 U Zymolyase/100 ul. This mixture was then incubated at 37 Deg C. for apx 15 min and the rest of the Qiagen miniprep kit protocol was followed according to manufactures instructions.

Transformation of electrocompetent E. coli host cells (DH12S) was performed using 4 μl of the extracted yeast plasmid DNA preparation and 50 μl of E. coli cells. The cells were electropulsed at 1.75 kV, 25 μF, and 400 ohms. Following electroporation, 0.5 ml LB was added and then the cells were plated in 10 μl and 30 μl aliquots on two LB AMP plates (LB broth (Lennox), 1.8% Bacto™ Agar (Difco), 100 mg/L Ampicillin).

The inserts DNA clones were subjected to sequence analysis. One clone containing the correct sequence is selected. Large-scale plasmid DNA is isolated using a commercially available kit (QIAGEN Plasmid Mega Kit, Qiagen, Valencia, Calif.) according to manufacturer's instructions.

The same process was used to prepare the truncated soluble human FGFR3_{III}c (S249W) Fc5 construct. A fragment represented a 5′ overlap with an optimized TPA leader in the pZMP31 vector sequence followed by sequence of human FGFR3_{III}c encoding a S249W point mutation, Fc5 sequence and a 3′ overlap with a linker sequence. For this fragment, the PCR amplification reactions used the 5′ oligonucleotides zc62560 (SEQ ID NO:20). (Forward primer to generate a PCR frag using FGFR3_{III}c as template. Fragment will generate a Ig D2 D3 form. Sequence of FGFR3_{III}c starts at D156 of SEQ ID NO:60, upstream of Ig D2. Fragment to be cloned into pZMP31 utilizing the opTPA leader seq.). The PCR was run with the 3′ oligonucleotides zc60566 (SEQ ID NO:82) and utilized MPET construct #1920 as template (SEQ ID NO:9).

This fragment was generated utilizing the same PCR thermocycles noted earlier and introduced into the BglII digested MVC 709 and MVC 710 vectors to generate a truncated soluble Human FGFR3IIIc (S249W) Fc5, a linker and downstream c-terminal scFv sequences specific for binding to VEGFA construct as described earlier.

The plasmids encoding full length or truncated soluble human FGFR3_{III}c (S249W) Fc5, a linker and down stream, c-term. scFv sequences specific for binding to VEGF-A were designated: FGFR3(143-375)(S249W)Fc5 c1094.1 pZMP31 (MVC 781) shown as SEQ ID NOS:57 and 58, FGFR3(23-375)(S249W)Fc5 c1094.1 pZMP31 (MVC 782) shown as SEQ ID NOS:59 and 60, FGFR3(143-375)(S249W)Fc5 c870e6 pZMP31 (MVC 783) shown as SEQ ID NOS:61 and 62 and FGFR3(23-375)(S249W)Fc5 c870e6 pZMP31 (MVC 784) shown as SEQ ID NOS:63 and 64. These plasmids were expressed transiently in 293F cells (Invitrogen, Carlsbad, Calif. Cat#R790-07). Mega prep DNA was prepared for each plasmid using a Qiagen Plasmid Mega Kit (Qiagen, Valencia, Calif.). 293F suspension cells were cultured in 293 Freestyle medium (Invitrogen, Carlsbad, Calif. Cat#12338-018) at 37° C., 6% CO2 in 3 L spinner flasks at 95 RPM. Fresh medium was added immediately prior to transfection to obtain a 1.5 liter working volume at a final density of 1×10E6 cells/mL. For each spinner, 2 mL of Lipofectamine 2000 (Invitrogen, Carlsbad, Calif. Cat#11668-019) was added to 20 mL Opti-MEM medium (Invitrogen, Carlsbad, Calif. Cat#31985-070) and 1.5 mg total Plasmid DNA was diluted in a separate tube of 20 mL Opti-MEM. Each tube was incubated separately at room temperature for 5 minutes, then combined and incubated together for an additional 30 minutes at room temperature with occasional gentle mixing. The lipid-DNA mixture was added to each spinner of 293F cells which were then returned to 37° C., 6% CO2 at 75 RPM. After approximately 96 hours, the conditioned medium was harvested, 0.2 μM filtered and submitted for protein purification.

### Example 16

### Purification Process

Conditioned media were delivered to purification as a 0.4 sterile filtered deliverable, containing 0.02% Sodium Azide. No further adjustments were made prior to loading the media to the affinity capture column.

For large scale purification, ˜10 Liter deliverable, an 87 mL bed by 2 centimeter diameter column of POROS A50 (protein A affinity resin) was employed for capture process purposes. For small scale purification (˜1-1.5 Liter deliverable) a 4 mL bed of POROS MabCapture A perfusion chromatography resin was utilized.

Prior to sample loading, the columns were equilibrated with 20 column volumes of Buffer A:10 mM Mono-Basic Sodium Phosphate, 10 mM Citric Acid Monohydrate, 250 mM [NH4]2S04 at pH 7.3 containing 0.02% sodium azide (W/v). Once equilibrated, the conditioned media was loaded at 20 mL per minute, for large scale process, or 10 mL per minute for small scale process. When the loading phase of process was completed, the unbound protein fraction was washed from the column with 10-20 column volumes of equilibration buffer. Elution of bound protein was accomplished via descending pH gradient, formed between the equilibration Buffer A and elution Buffer B of the following composition: 10 mM Mono-Basic Sodium Phosphate, 10 mM Citric Acid Monohydrate, 250 mM [NH4]2S04 at pH 3.0 containing 0.02% sodium azide (w/v). Elution of the large scale process was at 30 mL per minute flow rate while forming a 3 column volume gradient between Buffer A and Buffer B. Fractions (10 mL) were collected over 0.5 mL 2M Tris pH 8.0 buffer, contents were mixed immediately. Elution for small scale process employed the same buffers and a 4 column volume gradient from Buffer A to Buffer B at a flow rate of 5 mL per minute. Fractions (4 mL) were collected over 0.25 mL 2 M Tris pH 8.0 buffer. All eluate fractions were mixed immediately to ensure rapid pH neutralization.

All fractions with positive absorption at 280 nanometer wavelength were pooled and carried forward to a size exclusion chromatography step. Pooled protein was concentrated to 7 ml (large scale process) or 1.5 mL (small scale process) for size exclusion chromatography (SEC). SEC chromatography was utilized for buffer exchange purposes, as well as providing discrimination of small amounts of multimeric or aggregated species from the final product. The mobile phase for SEC and final protein formulation was 35 mM Sodium Phosphate, 120 mM Sodium Chloride at pH 7.3. Large scale SEC was performed on a Pharmacia Superdex 200 prep grade SEC column (26/60 format with 321 mL bed volume). Small scale SEC was performed on a Pharmacia Superdex 200 prep grade SEC column (16/60 format with 120 mL bed volume). Depending on process scale, a flow rate of either 2.5 mL per minute or 1.5 mL per minute, for either large or small scale respectively, was employed for the SEC step. Fractions under the main symmetric peak were pooled with emphasis on excluding any minor levels of high molecular weight materials from the final product. The final pool was sterile filtered at 0.2μ and aliquots were made and stored at −80° C. This is the same method is used for processing all FGFR receptors and bispecific binding compositions of a soluble FGF receptor (FGFR) and a VEGF-A antibody.

### Example 17

### ³H-Thymidine Proliferation Assay to Determine Neutralizing Activity of Soluble FGF Receptors on FGF-Stimulated HUVEC Cells

To screen for a neutralizing soluble FGF Receptors (FGFR) that inhibited the activity of various FGFs, a 3H-thymidine proliferation assay using human umbilical cord endothelial cells (HUVEC) was run. Recombinant human FGF1, 2, 4 and 6 (R&D Systems, Inc.) were used at 10 ng/ml in assay media (RPMI-1640, 1% FBS, 1 unit/ml Heparin and pyruvate). Soluble human FGFRs (R&D Systems and ZymoGenetics) were then titrated from 0.5-4 ug/ml (depending on the receptor) and serially diluted to 32-4 ng/ml in assay media. HUVEC were plated into 96-well flat-bottom plates (Costar) in a volume of 100 μL at a density of 2000 cells per well. The HUVEC proliferation assay was cultured for 2 days at 37° C., 5% CO2. HUVEC were then pulsed for 18 hr with 1 μCi per well of 3H-thymidine (Amersham, TRK120), which is incorporated into proliferating cells (all at 37° C., 5% CO2). The cells were harvested and counted on Packard TopCount NXT plate reader.

Results: All FGFRs tested neutralized FGF1 with similar activity as expected. FGFR1 and FGFR2 (R&D Systems) potently neutralized all FGFs. Whereas FGFR3 and FGFR4 (R&D Systems) are less potent in inhibiting FGF2, 4 and 6 activity. FGFR3A2258F (143_{—}375, S249W) (SEQ ID NO:10) and FGFR3A2256F (22_{—}375, S249W) (SEQ ID NO:2) potently neutralized FGFs1, 2, 4 and 6 with IC50 numbers similar to those of FGFR1.

TABLE 26

| IC50 (nM) | FGF1 | FGF2 | FGF4 | FGF6 |
|---|---|---|---|---|
| FGFR1 (R&D systems) | 0.63 | 0.92 | 0.53 | 0.42 |
| FGFR2 (R&D systems) | 0.42 | 0.85 | 0.48 | 0.3 |
| FGFR3 (R&D systems) | 0.53 | 5.5 | 1.06 | 9.96 |
| FGFR4 (R&D systems) | 0.85 | 18 | 4.15 | 19 |
| FGFR3 A2258F | 0.55 | 0.67 | 0.68 | 0.78 |
| (ZymoGenetics) | | | | |
| FGFR3 A2256F | 0.82 | 1 | 0.74 | 0.95 |
| (ZymoGenetics) | | | | |

### Example 18

### ³H-Thymidine Proliferation Assay to Determine Neutralizing Activity of Soluble FGF Receptors on FGF8b-Stimulated LNCap Cells

To screen for a neutralizing soluble FGF Receptors (FGFR) that inhibited the activity of FGF8b, a ³H-thymidine proliferation assay using human LNCap cells was run. Recombinant human FGF8b (R&D Systems, Inc.) were used at 200 to 1000 ng/ml in assay media (RPMI-1640, 1% BSA, ITS [Insulin-Transferrin and Selinium], L-glutamate and pyruvate [all from Invitrogen]). Soluble human FGFRs (R1-R4) from R&D Systems and FGFRs 3 and 2 from ZymoGenetics were then titrated from 1-2 ug/ml and serially diluted to 31-15 ng/ml in assay media. LNCap cells were plated into 96-well flat-bottom plates (Costar) in a volume of 100 μL at a density of 2500 cells per well. The LNCap proliferation assay was cultured for 3 days at 37° C., 5% CO₂. LNCap were then pulsed for 8 hr with 1 μCi per well of ³H-thymidine (Amersham, TRK120), which is incorporated into proliferating cells (all at 37° C., 5% CO₂). The cells were harvested and counted on Packard TopCount NXT plate reader.

Results: R&D System's FGFRs R2-R4, but not R1 neutralized FGF8b. Full length FGFR2 A2556F (22_{—}377) SEQ ID NO:24, A2557F (22377)(S252W) SEQ ID NO: 29, and A2558F (22_{—}377)(P253R) SEQ ID NO:33 did not neutralize FGF8b. Whereas truncated FGFR2A2559F (145_{—}377) SEQ ID NO:37, A2560F (145_{—}377)(S252W) SEQ ID NO:40, and A2561F (145_{—}377)(P253F) SEQ ID NO:42 did neutralize FGF8b similar to R&D Systems FGFR2. FGFR3 A2519F (143_{—}375)(P250R) SEQ ID NO:22, A2256F (23375)(S249W) SEQ ID NO:2, and A2258F (143_{—}375)(S249W) SEQ ID NO:10 neutralized FGF8b, but A2518F (143_{—}375) SEQ ID NO:19, A2257F (23_{—}375) SEQ ID NO:13, and A2259F (23_{—}375)(P250R) SEQ ID NO:15, did not.

TABLE 27

| FGFRs | Proliferation IC50 (nM) |
|---|---|
| R&D FGFR1 | ND |
| R&D FGFR2 | 0.29 |
| R&D FGFR3 | 0.79 |
| R&D FGFR4 | 0.13 |
| FGFR2 A2556F | ND |
| FGFR2 A2557F | ND |
| FGFR2 A2558F | ND |
| FGFR2 A2559F | 0.3 |
| FGFR2 A2560F | 0.01 |
| FGFR2 A2561F | 0.01 |
| FGFR3 A2518F | ND |
| FGFR3 A2519F | 0.01 |
| FGFR3 A2256F | 0.01 |
| FGFR3 A2257F | ND |
| FGFR3 A2258F | 0.02 |
| FGFR3 A2259F | ND |

ND, not determined

### Example 19

### ³H-Thymidine Proliferation Assay to Determine Neutralizing Activity of Soluble FGF Receptors on FGF8b-Stimulated MCF-7 Cells

To screen for a neutralizing soluble FGF Receptors (FGFR) that inhibit the activity of FGF8b, a ³H-thymidine proliferation assay using human MCF-7 cells are run. Recombinant human FGF8b (R&D Systems, Inc.) are at 100 ng/ml in assay media (RPMI-1640, 5% FBS, L-glutamate and pyruvate). Soluble human FGFR-Fcs (R1—R4) from R&D Systems and FGFRs 3 and 2 from ZymoGenetics are titrated from 10 ug/ml and serially diluted to 78 ng/ml in assay media. MCF-7 cells are plated into 96-well flat-bottom plates (Costar) in a volume of 100 μL at a density of 1250 cells per well. The MCF-7 proliferation assay are cultured for 4 days at 37° C., 5% CO₂. Cells are then be pulsed for 8 hr with 1 μCi per well of ³H-thymidine (Amersham, TRK120), which is incorporated into proliferating cells (all at 37° C., 5% CO₂). The cells are harvested and counted on Packard TopCount NXT plate reader.

### Example 20

### ³H-Thymidine Proliferation Assay to Determine Neutralizing Activity of sFGFR-Fc on FGF-9-Stimulated HCO Osteoblasts

To determine the neutralizing effect of sFGFR-Fc constructs on FGF-9-stimulated osteoblast proliferation, a ³H-thymidine assay was run. Human osteoblast cells were stimulated to grow with human FGF-9. FGFR-Fc proteins were added to the assay media at concentrations from 0.02 nM to 6 nM. Significant inhibition on osteoblast proliferation was observed, and IC50 was calculated for each protein.

Study Design: Human calvarial osteoblast cells (HCO; ScienCell, Carlsbad, Calif.) were stimulated with 1.2 nM human FGF-9 (R&D Systems, Minneapolis, Minn.). ObM assay media [osteoblast basal media (ObM, ScienCell) with 0.5% fetal bovine serum (FBS), 2 mM GlutaMax (Invitrogen, Carlsbad, Calif.), 1 mM sodium pyruvate, and 1× insulin-transferrin-selenium (Invitrogen)] was used as a negative control. Human FGFR1-Fc, FGFR2-Fc, FGFR3-Fc, and FGFR4-Fc (R&D Systems) were serially diluted in ObM assay media at 6 nM, 2 nM, 0.67 nM, 0.22 nM, 0.07 nM, and 0.02 nM. FGFR3-Fc mutants (ZymoGenetics, Seattle, Wash.) were also similarly diluted. HCO cells were plated in ObM supplemented with 5% FBS and osteoblast growth supplement (ObGS, ScienCell) in 96-well flat-bottom plates in a volume of 100 μL at a density of 1000 cells per well. The plates were incubated at 37° C., 5% CO₂ overnight. The cells were serum-starved with ObM assay media for 24 h, stimulated for 24 h with 1.2 nM FGF-9 with or without serially diluted FGFR-Fc, and pulsed for 24 h with 1 μCi per well of ³H-thymidine (GE Healthcare Biosciences, Piscataway, N.J.), which is incorporated into proliferating cells (all at 37° C., 5% CO₂). The cells were harvested and counted on a Packard TopCount NXT.

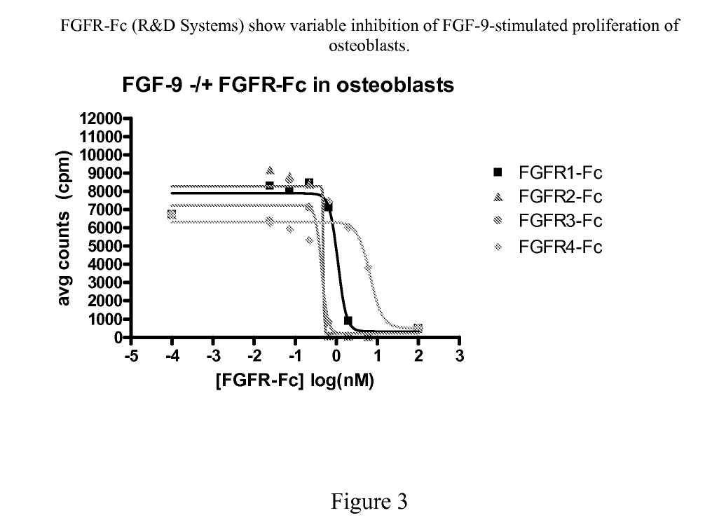

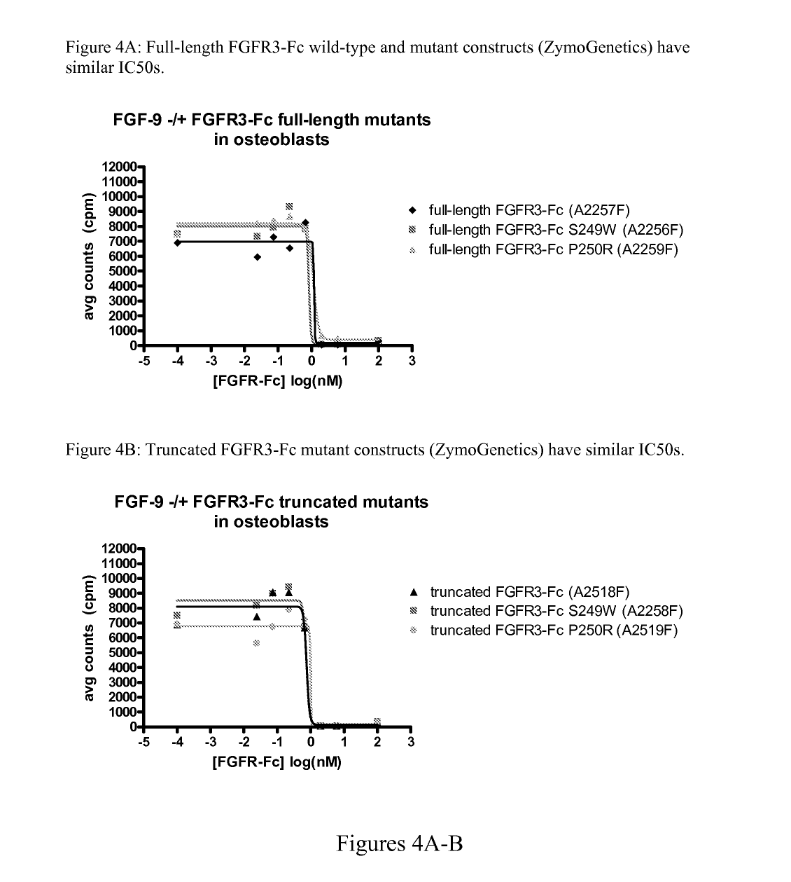

Results demonstrate that the mutant FGFR3-Fc constructs significantly inhibited human osteoblasts proliferation and had IC50s within a 3-fold range of the FGFR3-Fc from R&D Systems as shown in FIG. 3 and FIG. 2 illustrates that Full-length FGFR3-Fc wild-type and mutant constructs (ZymoGenetics) have similar IC50s.

TABLE 28
FGFR-Fc IC50s in the osteoblast proliferation assay.

| | Proliferation |
|---|---|
| FGFR-Fc | IC50 (nM) |
| FGFR1-Fc | 1.10 |
| FGFR2-Fc | 0.49 |
| FGFR3-Fc | 0.44 |
| FGFR4-Fc | 6.63 |
| Full-length FGFR3-Fc (A2257F) | 1.19 |
| Full-length FGFR3-Fc S249W (A2256F) | 0.84 |
| Full-length FGFR3-Fc P250R (A2259F) | 1.14 |
| Truncated FGFR3-Fc (A2518F) | 0.77 |
| Truncated FGFR3-Fc S249W (A2258F) | 0.75 |
| Truncated FGFR3-Fc P250R (A2519F) | 0.99 |

### Example 21

### Binding of FGFR-Fc Wild-Type and Mutant Constructs to FGF-8b and FGF-17

To determine the binding capability of FGFR-Fc to their respective ligands, an ELISA was run. Recombinant human FGF-8b or FGF-17 (R&D Systems) was plated at 100 nM on a Nunc Maxisorp 96-well plate for 1 h at room temperature with shaking. The plates were blocked with BLOTTO (Thermo Fisher Scientific, Rockford, Ill.) for 1 h at room temperature, then washed 5 times with ELISA C buffer (137 mM NaCl, 2.7 mM KCl, 7.2 mM Na₂HPO₄, 1.5 mM KH₂PO₄, 0.05% (v/w) polysorbate 20, pH 7.2). FGFR-Fcs were diluted to 100 nM with PBS/0.1×BLOTTO/10 ug/ml porcine heparin (Sigma, St. Louis, Mo.) and then 1:2 serial dilutions were made, ending at 0.10 nM. 100 μL of FGFR-Fcs were plated and incubated at 4° C. overnight. The following day, the plates were washed 5 times with ELISA C and then incubated with 2.5 μg/mL horseradish peroxidase-conjugated anti-human Fc antibody (Jackson ImmunoResearch Laboratories, West Grove, Pa.) for 1 h at room temperature with shaking. After 5 washes with ELISA C, 100 μL of OPD in citrate buffer (5 mg of o-phenylenediamine in 63 mM sodium citrate, 37 mM citric acid, pH 5.0, 0.03% H₂O₂) was added for detection. After 2-5 minutes, the reaction was stopped with 1 N H₂SO₄. The plates were read at 490 nm using SoftMaxPro software.

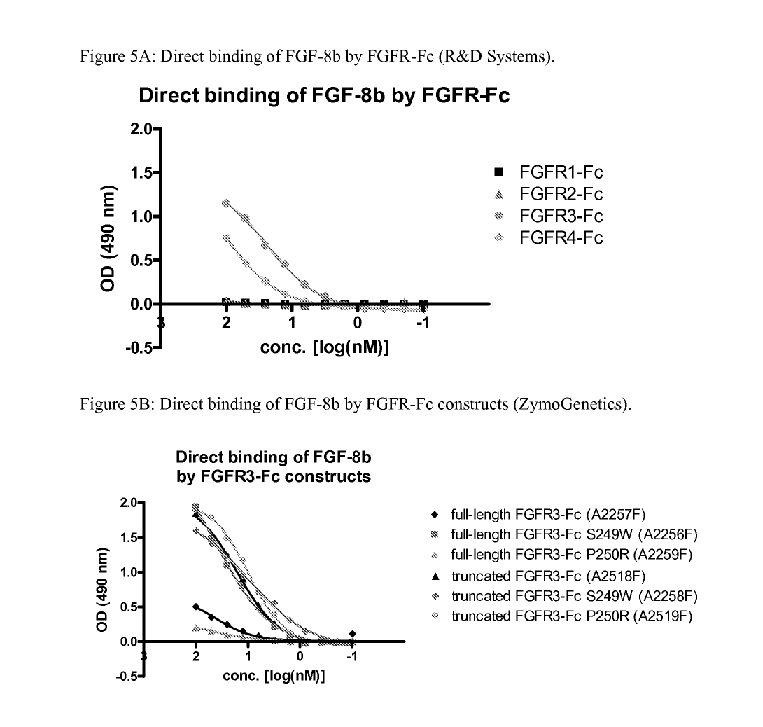

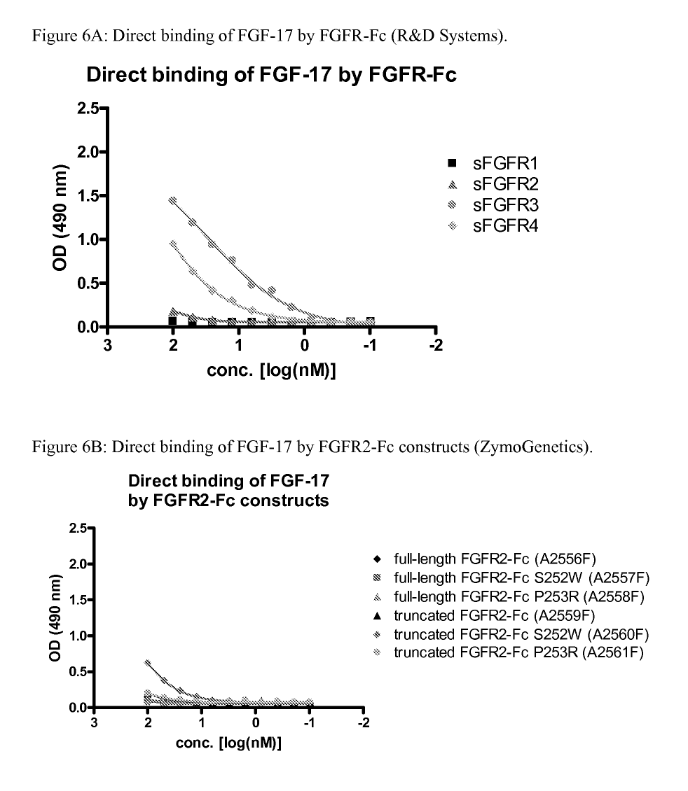

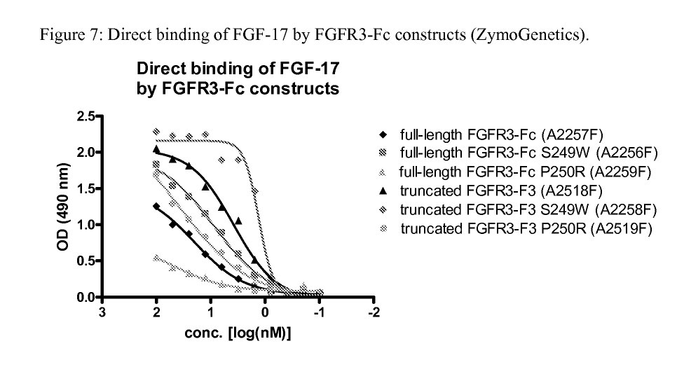

Results: While wild-type and mutant FGFR2-Fcs did not bind significantly to either FGF-8b or -17, there were some mutant FGFR3-Fc constructs that bound to both FGF-8b and FGF-17 to a greater degree than wild-type FGFR3-Fc as shown in FIGS. 5A, 5B, 6A, 6B and 7.

TABLE 29
Wild-type and mutant FGFR3-Fc constructs bind FGF-8b and FGF-17.

| | | FGF-17 |
|---|---|---|
| | FGF-8b binding | binding |
| FGFR-Fc | IC50 (nM) | IC50 (nM) |
| FGFR1-Fc | n/a | n/a |
| FGFR2-Fc | n/a | n/a |
| FGFR3-Fc | 23.4 | 30.1 |
| FGFR4-Fc | 142 | 317 |
| Full-length FGFR2-Fc (A2556F) | | n/a |
| Full-length FGFR2-Fc S252W (A2557F) | | n/a |
| Full-length FGFR2-Fc P253R (A2558F) | | n/a |
| Truncated FGFR2-Fc (A2559F) | | n/a |
| Truncated FGFR2-Fc S252W (A2560F) | | 223 |
| Truncated FGFR2-Fc P253R (A2561F) | | n/a |
| Full-length FGFR3-Fc (A2257F) | 41.9 | 20.1 |
| Full-length FGFR3-Fc S249W (A2256F) | 37.7 | 8.66 |
| Full-length FGFR3-Fc P250R (A2259F) | 52.6 | 45.3 |
| Truncated FGFR3-Fc (A2518F) | 16.4 | 3.79 |
| Truncated FGFR3-Fc S249W (A2258F) | 10.0 | 1.31 |
| Truncated FGFR3-Fc P250R (A2519F) | 9.7 | 37.6 |

### Example 22

### Measurement of Binding Affinities of FGF Receptors to FGF Ligands Via Surface Plasmon Resonance

Kinetic rate constants, equilibrium association constants, and equilibrium dissociation constants were measured for the interaction of soluble FGF receptors (FGFR) with FGF ligands via surface plasmon resonance. Various forms of FGFR3 were examined in this study. FGFR3 was produced with either two or three Ig-like domains, and with two possible point mutations (S249W or P250R). Each FGFR3 was produced as a dimeric Fc-fusion protein. For these studies, the Fc-tag was used to capture the FGFR molecule onto a Biacore chip previously immobilized with Protein-A. FGF ligands were flowed over the surface in a heparin-containing buffer. While the FGF ligands are monomers, they are believed to associate into dimers in the presence of heparin. Consequently, the bivalent analyte model was determined to be appropriate for these interactions.

Affinity Determination: FGF receptors were characterized for their binding affinity for FGF ligands. Association rate constants (kₐ (M⁻¹s⁻¹)) and dissociation rate constants (k_{d} (s⁻¹)) were measured for each interaction. The association rate constant is a value that reflects the rate of the ligand-receptor complex formation. The dissociation rate constant is a value that reflects the stability of this complex. Equilibrium binding affinity is typically expressed as either an equilibrium dissociation constant (K_{D} (M)) or an equilibrium association constant (K_{A} (M⁻¹)). K_{D} is obtained by dividing the dissociation rate constant by the association rate constant (k_{d}/kₐ), while K_{A} is obtained by dividing the association rate constant by the dissociation rate constant (kₐ/k_{d}). Molecules with similar K_{D} (or a similar K_{A}) can have widely variable association and dissociation rate constants. Consequently, measuring the kₐ and k_{d} as well as the K_{A} or K_{D} helps to more uniquely describe the affinity of the ligand-receptor interaction.

Materials and Methods: Binding kinetics and affinity studies were performed on a Biacore T100™ system (GE Healthcare, Piscataway, N.J.). Methods for the Biacore T100™ were programmed using Biacore T100™ Control Software, v 2.0. Since each of the FGF receptor molecules contained a human Fc domain, biotinylated protein-A (Thermo Fisher Scientific Inc, Rockford, Ill.) was used as a capture reagent for these studies. Biotinylated protein-A was diluted to concentration of 50 μg/mL in HBS-EP buffer (10 mM HEPES, 150 mM NaCl, 3 mM EDTA, 0.05% Surfactant P20; GE Healthcare, Piscataway, N.J.), and then captured to all four flow cells of a SA (streptavidin) sensor chip. A density of approximately 1100 RU was obtained for each flow cell. Each FGF receptor molecule was subsequently captured via protein-A onto a separate flow cell of the SA chip at an approximate density of 150-250 RU. The Biacore instrument measures the mass of protein bound to the sensor chip surface, and thus, capture of the receptor was verified for each cycle.

For kinetic binding studies, serial 1:5 dilutions of FGF ligands were prepared from 200 nM-0.06 nM. These samples were injected over the surface and allowed to specifically bind to the FGF receptor captured on the sensor chip. Injections of each ligand concentration were performed with an association time of 7 minutes and dissociation time of 15 minutes. Kinetic binding studies were performed with a flow rate of 50 μL/min. All binding experiments were performed at 25° C. in HBS-P buffer (10 mM HEPES, 150 mM NaCl, 0.05% Surfactant P20, pH 7.4; GE Healthcare, Piscataway, N.J.), containing 50 μg/mL heparin (Calbiochem, La Jolla, Calif.).

Between cycles, the flow cell was washed with 20 mM hydrochloric acid to regenerate the surface. This wash step removed both the captured FGF receptor and any bound FGF ligand from the protein-A surface, and allowed for the subsequent binding of the next test sample. Data was compiled using the Biacore T100™ Evaluation software (version 2.0). Data was processed by subtracting reference flow cell and blank injections. Baseline stability was assessed to ensure that the regeneration step provided a consistent binding surface throughout the sequence of injections. Duplicate injection curves were checked for reproducibility. Binding curves were globally fit to the bivalent analyte model.

Results: The bivalent analyte model was determined to be most appropriate for these interactions. This model measures two values for both kₐ (kₐ₁ and kₐ₂) and for k_{d} (k_{d1} and k_{d2}). The first set of values (kₐ₁ and k_{d1}) describes the monovalent kinetics of the interaction. The affinity reported for these samples was derived from these values, and is designated K_{D1} and K_{A1}. The second set of values (kₐ₂ and k_{d2}) refers to the avidity of the interaction and is not reported. While there was some trending in the residuals, the fit to the model was satisfactory to estimate binding affinity. The kinetics of binding interactions of the various FGFR3 molecules with FGF6 are detailed in Table 30. The affinity of the full-length FGFR molecule (23_{—}375) was similar to that of the two domain FGFR molecule (143_{—}375). The point mutations increased the affinity for FGF6, with the affinity of S249W>P250R >wild type. In general, this increase in affinity was primarily due to a slower dissociation rate constant.

TABLE 30
Binding Affinity for FGF6

| | kₐ₁ | k_{d1} | | |
|---|---|---|---|---|
| Description | (M⁻¹s⁻¹⁾ | (s⁻¹) | K_{D1} (M) | K_{A1} (M⁻¹) |
| FGFR3IIIc(23_375) C(Fc5) | 4.E+05 | 6.E−02 | 2.E−07 | 7.E+06 |
| FGFR3IIIc(23_375) (S249W) | 6.E+05 | 5.E−04 | 8.E−10 | 1.E+09 |
| C(Fc5) | | | | |
| FGFR3IIIc(23_375) (P250R) | 3.E+05 | 5.E−03 | 2.E−08 | 6.E+07 |
| C(Fc5) | | | | |
| FGFR3IIIc(143_375) C(Fc5) | 2.E+05 | 6.E−02 | 3.E−07 | 3.E+06 |
| FGFR3IIIc(143_375) (S249W) | 3.E+05 | 6.E−04 | 2.E−09 | 5.E+08 |
| C(Fc5) | | | | |
| FGFR3IIIc(143_375) (P250R) | 2.E+05 | 4.E−03 | 2.E−08 | 5.E+07 |
| C(Fc5) | | | | |

### Example 23

### ³H-Thymidine Proliferation Assay to Determine the Inhibition of Proliferation by FGFR-Fc on Tumor Cells

To determine the ability of FGFR-Fc to inhibit the proliferation of tumor cells, a ³H-thymidine assay was run. Caki-1 and DU145 tumor cells were plated were plated into 96-well flat-bottom plates at a density of 2000 cells per well and incubated at 37° C., 5% CO₂ overnight. The next day, FGFR-Fc constructs were serially diluted in RPMI 1640 (with 0.5% FBS, 1 mM sodium pyruvate, and 2 mM GlutaMAX) at 20, 10, and 5 μg/mL and plated onto the cells for 3 days at 37° C., 5% CO₂. The cells were pulsed for 24 h with 1 μCi per well of ³H-thymidine (GE Healthcare Biosciences, Piscataway, N.J.), which is incorporated into proliferating cells. The cells were harvested and counted on a Packard TopCount NXT.

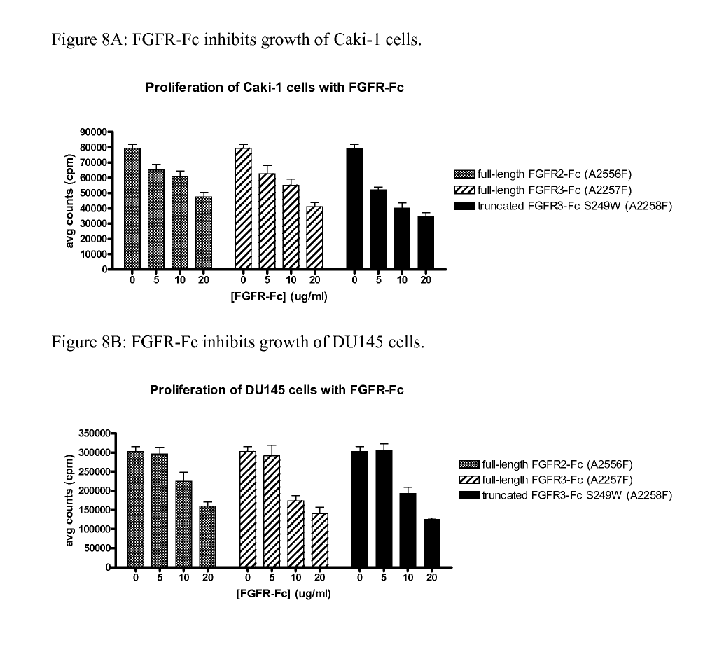

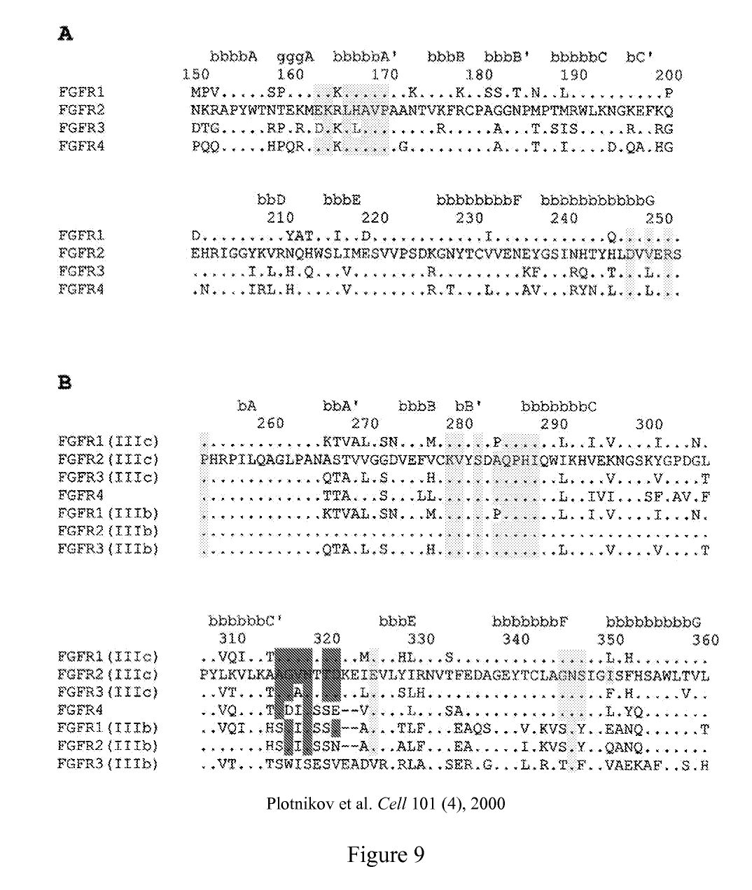

Results: At 20 μg/mL, the truncated FGFR3—FC S249W mutant inhibited both Caki-1 and DU145 cells to a slightly greater degree than either wild-type FGFR2-Fc or FGFR3-Fc. FIG. 8A demonstrates FGFR-Fc inhibits growth of Caki-1 cells and FIG. 8B demonstrates FGFR-Fc inhibits growth of DU145 cells.

TABLE 31
Percent inhibition of proliferation

| | Full-length | Full-length | Truncated |
|---|---|---|---|
| | FGFR2-Fc | FGFR3-Fc | FGFR3-Fc S249W |
| cells | A2256F | A2257F | A2258F |
| Caki-1 | 41% | 48% | 56% |
| DU145 | 47% | 53% | 59% |

### Example 24

### Inhibition of Endothelial Cell Sprouting by sFGFR-VEGF scFv Proteins

To test efficacy of the a bispecific binding protein comprising a VEGF-A antibody/soluble FGF receptor bispecific binding protein, an in vitro co-culture system of endothelial cells and pericytes are established as described (Darland et al, Dev Biol 264 (2003), 275). In this co-culture, HUVECs coated on Cytodex beads are co-cultured with human mesenchymal stem cells (Lonzo) in presence of EGM-2 complete media and D551 fibroblast conditioned media in fibrin gel. Either at start of the experiment or at Day 7 of the experiment, 0.04-50 nM of control anatgonist, VEGF-A antagonist or a bispecific binding protein comprising a VEGF-A antibody/soluble FGF receptor bispecific binding protein are added to the cultures. Cells are fixed on Day 8 after addition of antagonists using PFA. Cells are then stained by IHC using anti-smooth muscle cell actin (aSMA) or anti-PECAM antibodies to identify pericytes and endothelial cells respectively. In wells with control antagonist treatment, these cells form sprouts of endothelial cells protected by a covering of pericytes. In cells treated with anti-VEGF-A, a bispecific binding protein comprising a VEGF-A antibody/soluble FGF receptor bispecific binding protein, numbers of sprouts and length of the sprouts are reduced suggesting that the antagonist shows efficacy in this in vitro co-culture model.

Study Design: On Day 1, Cytodex-3 beads are coated with HUVECs and incubated overnight at 37° C., 5% CO₂. On Day 2, HUVEC beads (200 beads/well) are embedded in fibrin gel along with human mesenchymal stem cells (hMSC) (40,000 cels/well) in wells of a 24 well plate. A 1:1 mixture of EGM-2 complete media and D551 fibroblast media are added to these cells along with 2 ng/mL of HGF. Medium is replaced every two days till end of the experiment. Antagonists are added to the culture at Day 2 (from start of the co-culture) or at Day 7 (after co-culture formation). Cells are fixed in 4% PFA overnight six days after addition of antagonists. Cells are stained with anti-PECAM or anti-SMA antibodies followed by secondary antibody (fluorescent conjugated). Cells are then viewed by microscope and the numbers and lengths of sprouts counted manually for a representative set of 10 beads/well. The averages for the well are then calculated.

Results: In wells with control antagonist treatment, cells form sprouts of endothelial cells protected by a covering of pericytes. In cells treated with anti-VEGF-A or a bispecific binding protein comprising a VEGF-A antibody/soluble FGF receptor bispecific binding protein, reduction in numbers of sprouts and length of the sprouts suggest that the antagonist shows efficacy in this in vitro co-culture model.

### Example 25

### Prophylactic Treatment with sFGFR-Fc Proteins Inhibits Growth of A549 Lung Carcinoma Cells in Nu/Nu Mice

To test if the sFGFR proteins have activity on tumor growth in mice, groups of mice are injected s.c. with the A549 lung carcinoma tumors on Day 0. Groups of mice (n=10/gp) mice are then injected with 0.01 mg/Kg to 10 mg/Kg control reagent, sFGFR-Fc proteins 2-3×/week for 4 weeks, starting one day after tumor inoculation. Tumor volume is monitored 3×/week for 4 weeks. Significantly smaller tumors in mice injected with a sFGFR protein as compared to mice injected with control reagent, indicate efficacy of the antagonist for inhibition of tumor growth.

Study Design: Eight to ten-week old female Nu/Nu mice (Charles River Laboratories) are injected s.c. on the right flank with 2×10⁶ A549 cells on Day 0, Starting on Day 1, groups of mice (n=10/group) were injected i.p. with concentrations between 0.01 mg/Kg to 10 mg/Kg control reagent or sFGFR-Fc proteins 2-3×/week for 4 weeks. Tumor growth is monitored 3×/week for 4 weeks using caliper measurements. Tumor volume is calculated using the formula ½*(B)2*L (mm3) At the end of the study (24 hrs after last dose), mice are terminated and tumors weighed and submitted for histology. Tumors are fixed in NBF and are then tested for blood vessel density by immunohistochemistry using the MECA-32 antibody that is specific for mouse endothelial cells.

Results: Significantly smaller tumors in mice injected with a sFGFR protein as compared to mice injected with control reagent, indicate efficacy of the antagonist for inhibition of tumor growth.

### Example 26

### Therapeutic Treatment with sFGFR-Fc Proteins Inhibits Growth of A549 Lung Carcinoma Cells in Nu/Nu Mice

To test if the sFGFR proteins has activity on tumor growth in mice, groups of mice are injected s.c with the A549 lung carcinoma tumors on Day 0. When tumors reach a size of 200 mm³, groups of mice (n=10/gp) mice are injected with 0.01 mg/Kg-10 mg/Kg control reagent, or sFGFR-Fc proteins 2-3×/week for 4 weeks. Tumor volume is monitored 3×/week. Significantly smaller tumors in mice injected with sFGFR-Fc proteins, as compared to mice injected with control reagent, indicate efficacy of the antagonist for inhibition of tumor growth.

Study Design: Eight to ten-week old female Nu/Nu mice (Charles River Laboratories) are injected s.c. on the right flank with 2×10⁶ A549 cells on Day 0. When tumors reach a size of 200 mm³, groups of mice (n=10/group) are injected i.p. with 0.01 mg/Kg-10 mg/Kg control reagent, or sFGFR-Fc proteins 2-3×/week for 4 weeks. Tumor growth is monitored 3×/week using caliper measurements. Tumor volume is calculated using the formula ½*(B)2*L (mm3) At the end of the study (24 hrs after last dose), mice are terminated and tumors weighed. Tumors are also submitted for histological analysis for microvessel density.

Results: Significantly smaller tumors in mice injected with a sFGFR protein as compared to mice injected with control reagent, indicate efficacy of the antagonist for inhibition of tumor growth.

### Example 27

### Prophylactic Treatment with sFGFR-Fc Proteins Inhibits Growth of DU145 Prostate Cancer Cells in Nu/Nu Mice

To test if the sFGFR proteins has activity on tumor growth in mice, groups of mice are injected s.c with the DU145 prostate carcinoma tumors on Day 0. Groups of mice (n=10/gp) mice are then injected with 0.01 mg/Kg to 10 mg/Kg control reagent, sFGFR-Fc proteins 2-3×/week for 4 weeks, starting one day after tumor inoculation. Tumor volume is monitored 3×/week for 4 weeks. Significantly smaller tumors in mice injected with a sFGFR protein as compared to mice injected with control reagent, indicate efficacy of the antagonist for inhibition of tumor growth.

Study Design: Eight to ten-week old female Nu/Nu mice (Charles River Laboratories) are injected s.c. on the right flank with 2×10⁶ DU145 cells on Day 0, Starting on Day 1, groups of mice (n=10/group) were injected i.p. with concentrations between 0.01 mg/Kg to 10 mg/Kg control reagent or sFGFR-Fc proteins 2-3×/week for 4 weeks. Tumor growth is monitored 3×/week for 4 weeks using caliper measurements. Tumor volume is calculated using the formula ½*(B)2*L (mm3). At the end of the study (24 hrs after last dose), mice are terminated and tumors weighed and submitted for histology. Tumors are fixed in NBF and are then tested for blood vessel density by immunohistochemistry using the MECA-32 antibody that is specific for mouse endothelial cells.

Results: Significantly smaller tumors in mice injected with a sFGFR protein as compared to mice injected with control reagent, indicate efficacy of the antagonist for inhibition of tumor growth.

### Example 28

### Therapeutic Treatment with sFGFR-Fc Proteins Inhibits Growth of DU145 Prostate Carcinoma Cells in Nu/Nu Mice

To test if the sFGFR proteins has activity on tumor growth in mice, groups of mice are injected s.c with the DU145 prostate carcinoma tumors on Day 0. When tumors reach a size of 200 mm³, groups of mice (n=10/gp) mice are injected with 0.01 mg/Kg-10 mg/Kg control reagent, or sFGFR-Fc proteins 2-3×/week for 4 weeks. Tumor volume is monitored 3×/week. Significantly smaller tumors in mice injected with sFGFR-Fc proteins, as compared to mice injected with control reagent, indicate efficacy of the antagonist for inhibition of tumor growth.

Study Design: Eight to ten-week old female Nu/Nu mice (Charles River Laboratories) are injected s.c. on the right flank with 2×10⁶ DU145 cells on Day 0. When tumors reach a size of 200 mm³, groups of mice (n=10/group) are injected i.p. with 0.01 mg/Kg-10 mg/Kg control reagent, or sFGFR-Fc proteins 2-3×/week for 4 weeks. Tumor growth is monitored 3×/week using caliper measurements. Tumor volume is calculated using the formula ½*(B)2*L (mm3) At the end of the study (24 hrs after last dose), mice are terminated and tumors weighed. Tumors are also submitted for histological analysis for microvessel density.

Results: Significantly smaller tumors in mice injected with a sFGFR protein as compared to mice injected with control reagent, indicate efficacy of the antagonist for inhibition of tumor growth.

### Example 29

### Prophylactic Treatment with Bispecific Binding Proteins Inhibits Growth of A549 Lung Carcinoma Cells a Cells in Nu/Nu Mice

To test if the a bispecific binding protein comprising a VEGF-A antibody/soluble FGF receptor bispecific binding protein has activity on tumor growth in mice, groups of mice are injected s.c with the A549 lung carcinoma tumors on Day 0. Groups of mice (n=10/gp) mice are then injected with 0.01 mg/Kg to 10 mg/Kg control reagent, sFGFR-VEGF scFv fusion proteins 2-3×/week for 4 weeks, starting one day after tumor inoculation. Tumor volume is monitored 3×/week for 4 weeks. Significantly smaller tumors in mice injected with a a bispecific binding protein comprising a VEGF-A antibody/soluble FGF receptor bispecific binding protein as compared to mice injected with control reagent, indicate efficacy of the antagonist for inhibition of tumor growth.

Study Design: Eight to ten-week old female Nu/Nu mice (Charles River Laboratories) are injected s.c. on the right flank with 2×10⁶ A549 cells on Day 0, Starting on Day 1, groups of mice (n=10/group) were injected i.p. with concentrations between 0.01 mg/Kg to 10 mg/Kg control reagent or sFGFR-VEGF scFv fusion proteins 2-3×/week for 4 weeks. Tumor growth is monitored 3×/week for 4 weeks using caliper measurements. Tumor volume is calculated using the formula ½*(B)2*L (mm3) At the end of the study (24 hrs after last dose), mice are terminated and tumors weighed and submitted for histology. Tumors are fixed in NBF and are then tested for blood vessel density by immunohistochemistry using the MECA-32 antibody that is specific for mouse endothelial cells.

Results: Significantly smaller tumors in mice injected with a sFGFR-VEGF scFv fusion proteins as compared to mice injected with control reagent, indicate efficacy of the antagonist for inhibition of tumor growth.

### Example 30

### Therapeutic Treatment with Bispecific Binding Protein Inhibits Growth of A549 Lung Carcinoma Cells in Nu/Nu Mice

To test if the a bispecific binding protein comprising a VEGF-A antibody/soluble FGF receptor bispecific binding protein have activity on tumor growth in mice, groups of mice are injected s.c with the A549 lung carcinoma tumors on Day 0. When tumors reach a size of 200 mm³, groups of mice (n=10/gp) mice are injected with 0.01 mg/Kg-10 mg/Kg control reagent, or a bispecific binding protein comprising a VEGF-A antibody/soluble FGF receptor bispecific binding protein 2-3×/week for 4 weeks. Tumor volume is monitored 3×/week. Significantly smaller tumors in mice injected with a bispecific binding protein comprising a VEGF-A antibody/soluble FGF receptor bispecific binding protein, as compared to mice injected with control reagent, indicate efficacy of the antagonist for inhibition of tumor growth.

Study Design: Eight to ten-week old female Nu/Nu mice (Charles River Laboratories) are injected s.c. on the right flank with 2×10⁶ A549 cells on Day 0. When tumors reach a size of 200 mm³, groups of mice (n=10/group) are injected i.p. with 0.01 mg/Kg-10 mg/Kg control reagent, or a bispecific binding protein comprising a VEGF-A antibody/soluble FGF receptor bispecific binding protein 2-3×/week for 4 weeks. Tumor growth is monitored 3×/week using caliper measurements. Tumor volume is calculated using the formula ½*(B)2*L (mm3) At the end of the study (24 hrs after last dose), mice are terminated and tumors weighed. Tumors are also submitted for histological analysis for microvessel density.

Results: Significantly smaller tumors in mice injected with a bispecific binding protein comprising a VEGF-A antibody/soluble FGF receptor bispecific binding protein as compared to mice injected with control reagent, indicate efficacy of the antagonist for inhibition of tumor growth.

### Example 31

### Prophylactic Treatment with Bispecific Binding Protein Inhibits Growth of DU145 Prostate Cancer Cells in Nu/Nu Mice

To test if the bispecific binding protein comprising a VEGF-A antibody/soluble FGF receptor bispecific binding protein has activity on tumor growth in mice, groups of mice are injected s.c with the DU145 prostate carcinoma tumors on Day 0. Groups of mice (n=10/gp) mice are then injected with 0.01 mg/Kg to 10 mg/Kg control reagent, a bispecific binding protein comprising a VEGF-A antibody/soluble FGF receptor bispecific binding protein 2-3×/week for 4 weeks, starting one day after tumor inoculation. Tumor volume is monitored 3×/week for 4 weeks. Significantly smaller tumors in mice injected with a bispecific binding protein comprising a VEGF-A antibody/soluble FGF receptor bispecific binding protein as compared to mice injected with control reagent, indicate efficacy of the antagonist for inhibition of tumor growth.

Study Design: Eight to ten-week old female Nu/Nu mice (Charles River Laboratories) are injected s.c. on the right flank with 2×10⁶ DU145 cells on Day 0, Starting on Day 1, groups of mice (n=10/group) were injected i.p. with concentrations between 0.01 mg/Kg to 10 mg/Kg control reagent or a bispecific binding protein comprising a VEGF-A antibody/soluble FGF receptor bispecific binding protein 2-3×/week for 4 weeks. Tumor growth is monitored 3×/week for 4 weeks using caliper measurements. Tumor volume is calculated using the formula ½*(B)2*L (mm3) At the end of the study (24 hrs after last dose), mice are terminated and tumors weighed and submitted for histology. Tumors are fixed in NBF and are then tested for blood vessel density by immunohistochemistry using the MECA-32 antibody that is specific for mouse endothelial cells.

Results: Significantly smaller tumors in mice injected with a bispecific binding protein comprising a VEGF-A antibody/soluble FGF receptor bispecific binding protein as compared to mice injected with control reagent, indicate efficacy of the antagonist for inhibition of tumor growth.

### Example 32

### Therapeutic Treatment with Bispecific Binding Proteins Inhibits Growth of DU145 Prostate Carcinoma Cells in Nu/Nu Mice

To test if the bispecific binding protein comprising a VEGF-A antibody/soluble FGF receptor bispecific binding protein has activity on tumor growth in mice, groups of mice are injected s.c with the DU145 prostate carcinoma tumors on Day 0. When tumors reach a size of 200 mm³, groups of mice (n=10/gp) mice are injected with 0.01 mg/Kg-10 mg/Kg control reagent, or a bispecific binding protein comprising a VEGF-A antibody/soluble FGF receptor bispecific binding protein 2-3×/week for 4 weeks. Tumor volume is monitored 3×/week. Significantly smaller tumors in mice injected with a bispecific binding protein comprising a VEGF-A antibody/soluble FGF receptor bispecific binding protein, as compared to mice injected with control reagent, indicate efficacy of the antagonist for inhibition of tumor growth.

Study Design: Eight to ten-week old female Nu/Nu mice (Charles River Laboratories) are injected s.c. on the right flank with 2×10⁶ DU145 cells on Day 0. When tumors reach a size of 200 mm³, groups of mice (n=10/group) are injected i.p. with 0.01 mg/Kg-10 mg/Kg control reagent, or a bispecific binding protein comprising a VEGF-A antibody/soluble FGF receptor bispecific binding protein 2-3×/week for 4 weeks. Tumor growth is monitored 3×/week using caliper measurements. Tumor volume is calculated using the formula ½*(B)2*L (mm3) At the end of the study (24 hrs after last dose), mice are terminated and tumors weighed. Tumors are also submitted for histological analysis for microvessel density.

Results: Significantly smaller tumors in mice injected with a bispecific binding protein comprising a VEGF-A antibody/soluble FGF receptor bispecific binding protein as compared to mice injected with control reagent, indicate efficacy of the antagonist for inhibition of tumor growth.

From the foregoing, it will be appreciated that, although specific embodiments of the invention have been described herein for purposes of illustration, various modifications may be made without deviating from the spirit and scope of the invention. Accordingly, the invention is not limited except as by the appended claims. All publications, patents, and patent applications cited herein are hereby incorporated by reference in their entireties for all purposes.

1. A bispecific binding protein comprising an antibody moiety and a soluble receptor moiety, wherein the protein reduces the biological activity of both VEGF-A and FGF.

2. A bispecific binding protein comprising aVEGF-A antibody moiety and a FGF binding moiety of an FGF receptor, wherein the protein reduces the biological activity of both VEGF-A and FGF.

3. The bispecific binding protein of claim 2, wherein the soluble FGF receptor portion of the bispecific binding protein comprises an FGF receptor moiety of an FGFR3 or FGFR2.

4. A bispecific binding protein comprising a VEGF-A antibody/soluble FGF receptor bispecific binding protein comprising
an FGF receptor moiety that is an FGFR3 selected from the group consisting of SEQ ID NO:13, SEQ ID NO:2; SEQ ID NO:19; SEQ ID NO:10; SEQ ID NO:15; and SEQ ID NO:22; and
a VEGF-A antibody moiety selected from the group consisting of SEQ ID NO:44; SEQ ID NO:46; SEQ ID NO:52; and SEQ ID NO:70.

5. (canceled)

6. A polynucleotide encoding a bispecific protein wherein the polynucleotide encodes for a FGFR3 polypeptide selected from the group consisting of SEQ ID NO:13, SEQ ID NO:2; SEQ ID NO:19; SEQ ID NO:10; SEQ ID NO:15; and SEQ ID NO:22; and
a VEGF-A antibody polypeptide selected from the group consisting of SEQ ID NO:44; SEQ ID NO:46; SEQ ID NO:52; and SEQ ID NO:70.

7-10. (canceled)

11. A bispecific binding protein comprising a VEGF-A antibody/soluble FGF receptor bispecific binding protein comprising
an FGF receptor moiety that is an FGFR3 selected from the group consisting of SEQ ID NO:13; SEQ ID NO:2; SEQ ID NO:19; SEQ ID NO:10; SEQ ID NO:15; and SEQ ID NO:22; and
a VEGF-A antibody moiety selected from the group consisting of SEQ ID NO:48 and SEQ ID NO:50; SEQ ID NO:54 and SEQ ID NO:56; and SEQ ID NO:66 and SEQ ID NO:68.

12. (canceled)

13. A polynucleotide encoding a bispecific protein wherein the polynucleotide encodes for a FGFR3 polypeptide selected from the group consisting of SEQ ID NO:13, SEQ ID NO:2; SEQ ID NO:19; SEQ ID NO:10; SEQ ID NO:15; and SEQ ID NO:22; and
a VEGF-A antibody polypeptide selected from the group consisting of SEQ ID NO:48 and SEQ ID NO:50; SEQ ID NO:54 and SEQ ID NO:56; and SEQ ID NO:66 and SEQ ID NO:68.

14-17. (canceled)

18. A bispecific binding protein comprising an FGFR3 moiety and VEGF-A antibody moiety selected from the group consisting of SEQ ID NO:58; SEQ ID NO:60; SEQ ID NO:62; and SEQ ID NO:64.

19. (canceled)

20. A polynucleotide encoding a bispecific protein wherein the polynucleotide encodes for a FGFR3 moiety and VEGF-A antibody moiety selected from the group consisting of SEQ ID NO:58; SEQ ID NO:60; SEQ ID NO:62; and SEQ ID NO:64.

21-24. (canceled)

25. A bispecific binding protein comprising a VEGF-A antibody/soluble FGF receptor bispecific binding protein comprising
an FGF receptor moiety that is an FGFR2 selected from the group consisting of SEQ ID NO:24; SEQ ID NO:29; SEQ ID NO:33; SEQ ID NO:37; SEQ ID NO:40; and SEQ ID NO:42; and
a VEGF-A antibody moiety selected from the group consisting of SEQ ID NO:44; SEQ ID NO:46; SEQ ID NO:52; and SEQ ID NO:70.

26. (canceled)

27. A polynucleotide encoding a bispecific protein wherein the polynucleotide encodes for a FGFR2 polypeptide selected from the group consisting of SEQ ID NO:24; SEQ ID NO:29; SEQ ID NO:33; SEQ ID NO:37; SEQ ID NO:40; and SEQ ID NO:42; and
a VEGF-A antibody polypeptide selected from the group consisting of SEQ ID NO:44; SEQ ID NO:46; SEQ ID NO:52; and SEQ ID NO:70.

28-31. (canceled)

32. A bispecific binding protein comprising a VEGF-A antibody/soluble FGF receptor bispecific binding protein comprising
an FGF receptor moiety that is an FGFR2 selected from the group consisting of SEQ ID NO:24; SEQ ID NO:29; SEQ ID NO:33; SEQ ID NO:37; SEQ ID NO:40; and SEQ ID NO:42; and
a VEGF-A antibody moiety selected from the group consisting of SEQ ID NO:48 and SEQ ID NO:50; SEQ ID NO:54 and SEQ ID NO:56; and SEQ ID NO:66 and SEQ ID NO:68.

33. The bispecific binding protein of claims 4, 11, 18, 25 and 32, wherein the protein reduces the activity of FGF and VEGF-A.

34. A polynucleotide encoding a bispecific protein wherein the polynucleotide encodes for a FGFR2 polypeptide selected from the group consisting of SEQ ID NO:24; SEQ ID NO:29; SEQ ID NO:33; SEQ ID NO:37; SEQ ID NO:40; and SEQ ID NO:42; and
a VEGF-A antibody polypeptide selected from the group consisting of SEQ ID NO:48 and SEQ ID NO:50; SEQ ID NO:54 and SEQ ID NO:56; and SEQ ID NO:66 and SEQ ID NO:68.

35. An expression vector comprising the polynucleotide according to any of claims 6, 13, 20, 27 or 34.

36. A host cell comprising any one of the expression vectors of claim 35.

37. A method for producing a bispecific binding protein comprising:
culturing the host cell of claim 36 under conditions where in the bispecific binding protein is expressed; and
isolating the protein from the host cell.

38. A pharmaceutical composition comprising the bispecific binding protein of claims 4, 11, 18, 25 or 32 and pharmaceutically acceptable carrier.

39. A method of treating cancer in a subject comprising administering a effective amount of a bispecific binding protein according to any of claims 1, 2, 4, 11, 18, 25 or 32.

40. The method of claim 39, wherein the cancer is a solid tumor.

41. The method of claim 39, wherein the cancer is prostate cancer.

42. The method of claim 39, wherein the cancer is selected from the group consisting of lung cancer, a gastrointestinal tract cancer, gastrointestinal stromal tumor (GIST); pancreatic adenocarcinoma; pancreatic acinar cell carcinoma; a cancer of the small intestine; a cancer of the liver, breast cancer, cervical cancer, ovarian cancer, kidney cancer, skin cancer, glioblastoma and bone cancer.


SEQUENCE LISTING (89 sequences)

SEQ ID NO: 1 - DNA, 1863 bp, Artificial
  pZMP31solFGFR3IIIc(23_375)(S249W)Fc5
atggatgcaatgaagagagggctctgctgtgtgctgctgctgtgtggcgccgtcttcgtttcgctcagccaggaaatccatgccgagttgagacgcttccgtagagagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctggccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 2 - protein, 620 aa, Artificial
  Synthetic Construct
MDAMKRGLCCVLLLCGAVFVSLSQEIHAELRRFRRESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERWPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 60 bp, Artificial
  zc62552
caggaaatccatgccgagttgagacgcttccgtagagagtccttggggacggagcagcgc

SEQ ID NO: 4 - DNA, 50 bp, Artificial
  zc62557
cgcctgcaggatgggccggtgcggccagcgctccagcacgtccagcgtgt

SEQ ID NO: 5 - DNA, 51 bp, Artificial
  zc62556
tacacgctggacgtgctggagcgctggccgcaccggcccatcctgcaggcg

SEQ ID NO: 6 - DNA, 60 bp, Artificial
  zc62553
tgggcatgtgtgagttttgtctgaagatttgggctcgcctgcatacacactgcccgcctc

SEQ ID NO: 7 - DNA, 60 bp, Artificial
  zc62554
gtggaggctgacgaggcgggcagtgtgtatgcaggcgagcccaaatcttcagacaaaact

SEQ ID NO: 8 - DNA, 61 bp, Artificial
  zc62555
caaccccagagctgttttaaggcgcgcctctagattatttacccggagacagggagaggct

SEQ ID NO: 9 - DNA, 1503 bp, Artificial
  pZMP31solFGFR3IIIc(143_375)(S249W)Fc5
atggatgcaatgaagagagggctctgctgtgtgctgctgctgtgtggcgccgtcttcgtttcgctcagccaggaaatccatgccgagttgagacgcttccgtagagacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctggccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 10 - protein, 500 aa, Artificial
  Synthetic Construct
MDAMKRGLCCVLLLCGAVFVSLSQEIHAELRRFRRDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERWPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 60 bp, Artificial
  zc62560
caggaaatccatgccgagttgagacgcttccgtagagacacaggtgtggacacaggggcc

SEQ ID NO: 12 - DNA, 1863 bp, Artificial
  pZMP31solFGFR3IIIc(23_375)Fc5
atggatgcaatgaagagagggctctgctgtgtgctgctgctgtgtggcgccgtcttcgtttcgctcagccaggaaatccatgccgagttgagacgcttccgtagagagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 13 - protein, 620 aa, Artificial
  Synthetic Construct
MDAMKRGLCCVLLLCGAVFVSLSQEIHAELRRFRRESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 1863 bp, Artificial
  pZMP31solFGFR3IIIc(23_375)(P250R)Fc5
atggatgcaatgaagagagggctctgctgtgtgctgctgctgtgtggcgccgtcttcgtttcgctcagccaggaaatccatgccgagttgagacgcttccgtagagagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctcccggcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 15 - protein, 620 aa, Artificial
  Synthetic Construct
MDAMKRGLCCVLLLCGAVFVSLSQEIHAELRRFRRESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSRHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 51 bp, Artificial
  zc62558
ccccgcctgcaggatgggccggtgccgggagcgctccagcacgtccagcgt

SEQ ID NO: 17 - DNA, 51 bp, Artificial
  zc62559
acgctggacgtgctggagcgctcccggcaccggcccatcctgcaggcgggg

SEQ ID NO: 18 - DNA, 1503 bp, Artificial
  pZMP31solFGFR3IIIc(143_375)Fc5
atggatgcaatgaagagagggctctgctgtgtgctgctgctgtgtggcgccgtcttcgtttcgctcagccaggaaatccatgccgagttgagacgcttccgtagagacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 19 - protein, 500 aa, Artificial
  Synthetic Construct
MDAMKRGLCCVLLLCGAVFVSLSQEIHAELRRFRRDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 60 bp, Artificial; identical to SEQ ID NO: 11
  zc62560

SEQ ID NO: 21 - DNA, 1503 bp, Artificial
  pZMP31solFGFR3IIIc(143_375)(P250R)Fc5
atggatgcaatgaagagagggctctgctgtgtgctgctgctgtgtggcgccgtcttcgtttcgctcagccaggaaatccatgccgagttgagacgcttccgtagagacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctcccggcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 22 - protein, 500 aa, Artificial
  Synthetic Construct
MDAMKRGLCCVLLLCGAVFVSLSQEIHAELRRFRRDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSRHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1872 bp, Artificial
  pZMP31solFGFR2alphaIIIc(22_377)Fc5
atggatgcaatgaagagagggctctgctgtgtgctgctgctgtgtggcgccgtcttcgtttcgctcagccaggaaatccatgccgagttgagacgcttccgtagacggccctccttcagtttagttgaggataccacattagagccagaagagccaccaaccaaataccaaatctctcaaccagaagtgtacgtggctgcaccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggaggagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 24 - protein, 623 aa, Artificial
  Synthetic Construct
MDAMKRGLCCVLLLCGAVFVSLSQEIHAELRRFRRRPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 60 bp, Artificial
  zc62932
caggaaatccatgccgagttgagacgcttccgtagacggccctccttcagtttagttgag

SEQ ID NO: 26 - DNA, 60 bp, Artificial
  zc62933
tgggcatgtgtgagttttgtctgaagatttgggctcctccaggtagtctggggaagctgt

SEQ ID NO: 27 - DNA, 60 bp, Artificial
  zc62934
gaaaaggagattacagcttccccagactacctggaggagcccaaatcttcagacaaaact

SEQ ID NO: 28 - DNA, 1872 bp, Artificial
  pZMP31solFGFR2alphaIIIc(22_377)(S252W)Fc5
atggatgcaatgaagagagggctctgctgtgtgctgctgctgtgtggcgccgtcttcgtttcgctcagccaggaaatccatgccgagttgagacgcttccgtagacggccctccttcagtttagttgaggataccacattagagccagaagagccaccaaccaaataccaaatctctcaaccagaagtgtacgtggctgcaccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatggcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggaggagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 29 - protein, 623 aa, Artificial
  Synthetic Construct
MDAMKRGLCCVLLLCGAVFVSLSQEIHAELRRFRRRPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERWPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - DNA, 51 bp, Artificial
  zc62937
ggcttggaggatgggccggtgaggccatcgctccacaacatccaggtggta

SEQ ID NO: 31 - DNA, 51 bp, Artificial
  zc62936
taccacctggatgttgtggagcgatggcctcaccggcccatcctccaagcc

SEQ ID NO: 32 - DNA, 1872 bp, Artificial
  pZMP31solFGFR2alphaIIIc(22_377)(P253R)Fc5
atggatgcaatgaagagagggctctgctgtgtgctgctgctgtgtggcgccgtcttcgtttcgctcagccaggaaatccatgccgagttgagacgcttccgtagacggccctccttcagtttagttgaggataccacattagagccagaagagccaccaaccaaataccaaatctctcaaccagaagtgtacgtggctgcaccaggggagtcgctagaggtgcgctgcctgttgaaagatgccgccgtgatcagttggactaaggatggggtgcacttggggcccaacaataggacagtgcttattggggagtacttgcagataaagggcgccacgcctagagactccggcctctatgcttgtactgccagtaggactgtagacagtgaaacttggtacttcatggtgaatgtcacagatgccatctcatccggagatgatgaggatgacaccgatggtgcggaagattttgtcagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcgtcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggaggagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 33 - protein, 623 aa, Artificial [sequence omitted]

SEQ ID NO: 34 - DNA, 51 bp, Artificial
  zc62939
tccggcttggaggatgggccggtgacgcgatcgctccacaacatccaggtg

SEQ ID NO: 35 - DNA, 51 bp, Artificial
  zc62938
cacctggatgttgtggagcgatcgcgtcaccggcccatcctccaagccgga

SEQ ID NO: 36 - DNA, 1503 bp, Artificial
  pZMP31solFGFR2alphaIIIc(145_377)Fc5
atggatgcaatgaagagagggctctgctgtgtgctgctgctgtgtggcgccgtcttcgtttcgctcagccaggaaatccatgccgagttgagacgcttccgtagaagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggaggagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 37 - protein, 500 aa, Artificial
  Synthetic Construct
MDAMKRGLCCVLLLCGAVFVSLSQEIHAELRRFRRSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - DNA, 60 bp, Artificial
  zc62935
caggaaatccatgccgagttgagacgcttccgtagaagtgagaacagtaacaacaagaga

SEQ ID NO: 39 - DNA, 1503 bp, Artificial
  pZMP31solFGFR2alphaIIIc(145_377)(S252W)Fc5
atggatgcaatgaagagagggctctgctgtgtgctgctgctgtgtggcgccgtcttcgtttcgctcagccaggaaatccatgccgagttgagacgcttccgtagaagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatggcctcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggaggagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 40 - protein, 500 aa, Artificial
  Synthetic Construct
MDAMKRGLCCVLLLCGAVFVSLSQEIHAELRRFRRSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERWPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 1503 bp, Artificial
  pZMP31solFGFR2alphaIIIc(145_377)(P253R)Fc5
atggatgcaatgaagagagggctctgctgtgtgctgctgctgtgtggcgccgtcttcgtttcgctcagccaggaaatccatgccgagttgagacgcttccgtagaagtgagaacagtaacaacaagagagcaccatactggaccaacacagaaaagatggaaaagcggctccatgctgtgcctgcggccaacactgtcaagtttcgctgcccagccggggggaacccaatgccaaccatgcggtggctgaaaaacgggaaggagtttaagcaggagcatcgcattggaggctacaaggtacgaaaccagcactggagcctcattatggaaagtgtggtcccatctgacaagggaaattatacctgtgtggtggagaatgaatacgggtccatcaatcacacgtaccacctggatgttgtggagcgatcgcgtcaccggcccatcctccaagccggactgccggcaaatgcctccacagtggtcggaggagacgtagagtttgtctgcaaggtttacagtgatgcccagccccacatccagtggatcaagcacgtggaaaagaacggcagtaaatacgggcccgacgggctgccctacctcaaggttctcaaggccgccggtgttaacaccacggacaaagagattgaggttctctatattcggaatgtaacttttgaggacgctggggaatatacgtgcttggcgggtaattctattgggatatcctttcactctgcatggttgacagttctgccagcgcctggaagagaaaaggagattacagcttccccagactacctggaggagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaataa

SEQ ID NO: 42 - protein, 500 aa, Artificial
  Synthetic Construct
MDAMKRGLCCVLLLCGAVFVSLSQEIHAELRRFRRSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSRHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 744 bp, Artificial
  c870.1 e6 scFv
gaagttcaattgttagagtctggtggcggtcttgttcagcctggtggttctttacgtctttcttgcgctgcttccggattcactttctctggttacattatgtggtgggttcgccaagctcctggtaaaggtttggagtgggtttctgttatctctccttctggtggcgatacttggtatgctgactccgttaaaggtcgcttcactatctctagagacaactctaagaatactctctacttacagatgaacagcttaagggctgaggacacggccgtgtattactgtgccacagcgggagactactggggccagggcaccctggtcaccgtctcaagcggtggtggtggttctggcggcggcggctccggcgggggtggaagtggtggtggtggttctggtggtggtggttctcagagcgtgttgactcagccaccctcagcgtctgggacccccgggcagagggtcaccatctcttgttctggaagcagctccaacatcggaaggaatcctgtaaactggtaccagcagctcccaggaacggcccccaaactcctcatctatggtgataatcagcggccctcaggggtccctgaccgattctctggctccaaatctggcacctcagcctccctggccatcagtgggctccagtctgaggatgaggctgattattactgtgcagcatgggatgacagcctgaatggtgtggtattcggcggagggaccaagctgaccgtccta

SEQ ID NO: 44 - protein, 248 aa, Artificial
  Synthetic Construct
EVQLLESGGGLVQPGGSLRLSCAASGFTFSGYIMWWVRQAPGKGLEWVSVISPSGGDTWYADSVKGRFTISRDNSKNTLYLQMNSLRAEDTAVYYCATAGDYWGQGTLVTVSSGGGGSGGGGSGGGGSGGGGSGGGGSQSVLTQPPSASGTPGQRVTISCSGSSSNIGRNPVNWYQQLPGTAPKLLIYGDNQRPSGVPDRFSGSKSGTSASLAISGLQSEDEADYYCAAWDDSLNGVVFGGGTKLTVL

SEQ ID NO: 45 - DNA, 744 bp, Artificial
  1094.1 scFv
gaagttcaattgttagagtctggtggcggtcttgttcagcctggtggttctttacgtctttcttgcgctgcttccggattcactttctctggttacattatgtggtgggttcgccaagctcctggtaaaggtttggagtgggtttctgttatctctccttctggtggcgatacttggtatgctgactccgttaaaggtcgcttcactatctctagagacaactctaagaatactctctacttgcagatgaacagcttaagggctgaggacacggccgtgtattactgtgccacagcgggagactactggggccagggcaccctggtcactgtctcaagcggtggtggtggttctggcggcggcggctccggcgggggtggaagtggtggtggtggttctggtggtggtggttctcagagcgaattgactcagccaccctcagcgtctgggacccccgggcagggggtcaccatctcttgttctggaagtagttccaacatcggaagtaatactgttcagtggtaccagcagttcccaggaagggcccccaaactcctcatctatagtaataatcggcggccctcaggggtccctgaccgattctctggttccaagtctggcacctcagcctccctggccatcagtgggctccggtccgaggatgaagctgattattactgtgcagcatgggatgacagcctgagtgtcgtggtattcggcggagggaccaagctgaccgtccta

SEQ ID NO: 46 - protein, 248 aa, Artificial
  Synthetic Construct
EVQLLESGGGLVQPGGSLRLSCAASGFTFSGYIMWWVRQAPGKGLEWVSVISPSGGDTWYADSVKGRFTISRDNSKNTLYLQMNSLRAEDTAVYYCATAGDYWGQGTLVTVSSGGGGSGGGGSGGGGSGGGGSGGGGSQSELTQPPSASGTPGQGVTISCSGSSSNIGSNTVQWYQQFPGRAPKLLIYSNNRRPSGVPDRFSGSKSGTSASLAISGLRSEDEADYYCAAWDDSLSVVVFGGGTKLTVL

SEQ ID NO: 47 - DNA, 331 bp, Homo Sapien
cagagcgaattgactcagccaccctcagcgtctgggacccccgggcagagggtcaccatctcttgttctggaagcagctccaacatcggaaggaatcctgtaaactggtaccagcacctcccaggaacggcccccaaactcctcatctatggtgataatcagcggccctcaggggtccctgaccgattctctggctccaggtctggcacctcagcctccctggccatcagtgggctccagtctgaggatgaggctgattattactgtgcagcatgggatgacagcctgaatggtgtggtattcggcggagggaccaagctgaccgtcctag

SEQ ID NO: 48 - protein, 110 aa, Homo sapien
QSELTQPPSASGTPGQRVTISCSGSSSNIGRNPVNWYQHLPGTAPKLLIYGDNQRPSGVPDRFSGSRSGTSASLAISGLQSEDEADYYCAAWDDSLNGVVFGGGTKLTVL

SEQ ID NO: 49 - DNA, 340 bp, Homo sapien
gaagttcaattgttagagtctggtggcggtcttgttcagcctggtggttctttacgtctttcttgcgctgcttccggattcactttctctggttacattatgtggtgggttcgccaagctcctggtaaaggtttggagtgggtttctgttatctctccttctggtggcgatacttggtatgctgactccgttaaaggtcgcttcactatctctagagacaactctaagaatactctctacttacagatgaacagcttaagggctgaggacacggccgtgtattactgtgccacagcgggagactactggggccagggcaccctggtcaccgtctcaagcg

SEQ ID NO: 50 - protein, 113 aa, Homo sapien
EVQLLESGGGLVQPGGSLRLSCAASGFTFSGYIMWWVRQAPGKGLEWVSVISPSGGDTWYADSVKGRFTISRDNSKNTLYLQMNSLRAEDTAVYYCATAGDYWGQGTLVTVSS

SEQ ID NO: 51 - DNA, 744 bp, Artificial
  c870.1scFv cDNA
gaagttcaattgttagagtctggtggcggtcttgttcagcctggtggttctttacgtctttcttgcgctgcttccggattcactttctctggttacattatgtggtgggttcgccaagctcctggtaaaggtttggagtgggtttctgttatctctccttctggtggcgatacttggtatgctgactccgttaaaggtcgcttcactatctctagagacaactctaagaatactctctacttacagatgaacagcttaagggctgaggacacggccgtgtattactgtgccacagcgggagactactggggccagggcaccctggtcaccgtctcaagcggtggtggtggttctggcggcggcggctccggcgggggtggaagtggtggtggtggttctggtggtggtggttctcagagcgaattgactcagccaccctcagcgtctgggacccccgggcagagggtcaccatctcttgttctggaagcagctccaacatcggaaggaatcctgtaaactggtaccagcacctcccaggaacggcccccaaactcctcatctatggtgataatcagcggccctcaggggtccctgaccgattctctggctccaggtctggcacctcagcctccctggccatcagtgggctccagtctgaggatgaggctgattattactgtgcagcatgggatgacagcctgaatggtgtggtattcggcggagggaccaagctgaccgtccta

SEQ ID NO: 52 - protein, 248 aa, Artificial
  c870.1 scFv
EVGLLESGGGLVQPGGSLRLSCAASGFTFSGYIMWWVRQAPGKGLEWVSVISPSGGDTWYADSVKGRFTISRDNSKNTLYLQMNSLRAEDTAVYYCATAGDYWGQGTLVTVSSGGGGSGGGGSGGGGSGGGGSGGGGSQSELTQPPSASGTPGQRVTISCSGSSSNIGRNPVNWYQHLPGTAPKLLIYGDNQRPSGVPDRFSGSRSGTSASLAISGLQSEDEADYYCAAWDDSLNGVVFGGGTKLTVL

SEQ ID NO: 53 - DNA, 331 bp, Homo sapien
cagagcgaattgactcagccaccctcagcgtctgggacccccgggcagggggtcaccatctcttgttctggaagtagttccaacatcggaagtaatactgttcagtggtaccagcagttcccaggaagggcccccaaactcctcatctatagtaataatcggcggccctcaggggtccctgaccgattctctggttccaagtctggcacctcagcctccctggccatcagtgggctccggtccgaggattaggctgattattactgtgcagcatgggatgacagcctgagtgtcgtggtattcggcggagggaccaagctgaccgtcctag

SEQ ID NO: 54 - protein, 110 aa, Homo sapien
QSELTQPPSASGTPGQGVTISCSGSSSNIGSNTVQWYQQFPGRAPKLLIYSNNRRPSGVPDRFSGSKSGTSASLAISGLRSEDQADYYCAAWDDSLSVVVFGGGTKLTVL

SEQ ID NO: 55 - DNA, 340 bp, Homo sapien
gaagttcaattgttagagtctggtggcggtcttgttcagcctggtggttctttacgtctttcttgcgctgcttccggattcactttctctggttacattatgtggtgggttcgccaagctcctggtaaaggtttggagtgggtttctgttatctctccttctggtggcgatacttggtatgctgactccgttaaaggtcgcttcactatctctagagacaactctaagaatactctctacttgcagatgaacagcttaagggctgaggacacggccgtgtattactgtgccacagcgggagactactggggccagggcaccctggtcactgtctcaagcg

SEQ ID NO: 56 - protein, 114 aa, Homo sapien
EVQLLESGGGLVQPGGSLRLSCAASGFTFSGYIMWWVRQAPGKGLEWVSVISPSGGDTWYADSVKGRFTISRDNSKNTLYLLQMNSLRAEDTAVYYCATAGDYWGQGTLVTVSS

SEQ ID NO: 57 - DNA, 2274 bp, Artificial
  FGFR3(143-375)(S249W)Fc5 c1094.1 pZMP31
atggatgcaatgaagagagggctctgctgtgtgctgctgctgtgtggcgccgtcttcgtttcgctcagccaggaaatccatgccgagttgagacgcttccgtagagacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctggccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcgggggttcgggtggaggaggttctgaagttcaattgttagagtctggtggcggtcttgttcagcctggtggttctttacgtctttcttgcgctgcttccggattcactttctctggttacattatgtggtgggttcgccaagctcctggtaaaggtttggagtgggtttctgttatctctccttctggtggcgatacttggtatgctgactccgttaaaggtcgcttcactatctctagagacaactctaagaatactctctacttgcagatgaacagcttaagggctgaggacacggccgtgtattactgtgccacagcgggagactactggggccagggcaccctggtcactgtctcaagcggtggtggtggttctggcggcggcggctccggcgggggtggaagtggtggtggtggttctggtggtggtggttctcagagcgaattgactcagccaccctcagcgtctgggacccccgggcagggggtcaccatctcttgttctggaagtagttccaacatcggaagtaatactgttcagtggtaccagcagttcccaggaagggcccccaaactcctcatctatagtaataatcggcggccctcaggggtccctgaccgattctctggttccaagtctggcacctcagcctccctggccatcagtgggctccggtccgaggatgaagctgattattactgtgcagcatgggatgacagcctgagtgtcgtggtattcggcggagggaccaagctgaccgtcctataa

SEQ ID NO: 58 - protein, 757 aa, Artificial
  Synthetic Construct
MDAMKRGLCCVLLLCGAVFVSLSQEIHAELRRFRRDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERWPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFTFSGYIMWWVRQAPGKGLEWVSVISPSGGDTWYADSVKGRFTISRDNSKNTLYLQMNSLRAEDTAVYYCATAGDYWGQGTLVTVSSGGGGSGGGGSGGGGSGGGGSGGGGSQSELTQPPSASGTPGQGVTISCSGSSSNIGSNTVQWYQQFPGRAPKLLIYSNNRRPSGVPDRFSGSKSGTSASLAISGLRSEDEADYYCAAWDDSLSVVVFGGGTKLTVL

SEQ ID NO: 59 - DNA, 2634 bp, Artificial
  FGFR3(23-375)(S249W)Fc5 c1094.1 pZMP31
atggatgcaatgaagagagggctctgctgtgtgctgctgctgtgtggcgccgtcttcgtttcgctcagccaggaaatccatgccgagttgagacgcttccgtagagagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctggccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcgggggttcgggtggaggaggttctgaagttcaattgttagagtctggtggcggtcttgttcagcctggtggttctttacgtctttcttgcgctgcttccggattcactttctctggttacattatgtggtgggttcgccaagctcctggtaaaggtttggagtgggtttctgttatctctccttctggtggcgatacttggtatgctgactccgttaaaggtcgcttcactatctctagagacaactctaagaatactctctacttgcagatgaacagcttaagggctgaggacacggccgtgtattactgtgccacagcgggagactactggggccagggcaccctggtcactgtctcaagcggtggtggtggttctggcggcggcggctccggcgggggtggaagtggtggtggtggttctggtggtggtggttctcagagcgaattgactcagccaccctcagcgtctgggacccccgggcagggggtcaccatctcttgttctggaagtagttccaacatcggaagtaatactgttcagtggtaccagcagttcccaggaagggcccccaaactcctcatctatagtaataatcggcggccctcaggggtccctgaccgattctctggttccaagtctggcacctcagcctccctggccatcagtgggctccggtccgaggatgaagctgattattactgtgcagcatgggatgacagcctgagtgtcgtggtattcggcggagggaccaagctgaccgtcctataa

SEQ ID NO: 60 - protein, 877 aa, Artificial
  Synthetic Construct
MDAMKRGLCCVLLLCGAVFVSLSQEIHAELRRFRRESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERWPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFTFSGYIMWWVRQAPGKGLEWVSVISPSGGDTWYADSVKGRFTISRDNSKNTLYLQMNSLRAEDTAVYYCATAGDYWGQGTLVTVSSGGGGSGGGGSGGGGSGGGGSGGGGSQSELTQPPSASGTPGQGVTISCSGSSSNIGSNTVQWYQQFPGRAPKLLIYSNNRRPSGVPDRFSGSKSGTSASLAISGLRSEDEADYYCAAWDDSLSVVVFGGGTKLTVL

SEQ ID NO: 61 - DNA, 2274 bp, Artificial
  FGFR3(143-375)(S249W)Fc5 c870e6 pZMP31
atggatgcaatgaagagagggctctgctgtgtgctgctgctgtgtggcgccgtcttcgtttcgctcagccaggaaatccatgccgagttgagacgcttccgtagagacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctggccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcgggggttcgggtggaggaggttctgaagttcaattgttagagtctggtggcggtcttgttcagcctggtggttctttacgtctttcttgcgctgcttccggattcactttctctggttacattatgtggtgggttcgccaagctcctggtaaaggtttggagtgggtttctgttatctctccttctggtggcgatacttggtatgctgactccgttaaaggtcgcttcactatctctagagacaactctaagaatactctctacttacagatgaacagcttaagggctgaggacacggccgtgtattactgtgccacagcgggagactactggggccagggcaccctggtcaccgtctcaagcggtggtggtggttctggcggcggcggctccggcgggggtggaagtggtggtggtggttctggtggtggtggttctcagagcgtgttgactcagccaccctcagcgtctgggacccccgggcagagggtcaccatctcttgttctggaagcagctccaacatcggaaggaatcctgtaaactggtaccagcagctcccaggaacggcccccaaactcctcatctatggtgataatcagcggccctcaggggtccctgaccgattctctggctccaaatctggcacctcagcctccctggccatcagtgggctccagtctgaggatgaggctgattattactgtgcagcatgggatgacagcctgaatggtgtggtattcggcggagggaccaagctgaccgtcctataa

SEQ ID NO: 62 - protein, 757 aa, Artificial
  Synthetic Construct
MDAMKRGLCCVLLLCGAVFVSLSQEIHAELRRFRRDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERWPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFTFSGYIMWWVRQAPGKGLEWVSVISPSGGDTWYADSVKGRFTISRDNSKNTLYLQMNSLRAEDTAVYYCATAGDYWGQGTLVTVSSGGGGSGGGGSGGGGSGGGGSGGGGSQSVLTQPPSASGTPGQRVTISCSGSSSNIGRNPVNWYQQLPGTAPKLLIYGDNQRPSGVPDRFSGSKSGTSASLAISGLQSEDEADYYCAAWDDSLNGVVFGGGTKLTVL

SEQ ID NO: 63 - DNA, 2634 bp, Artificial
  FGFR3(23-375)(S249W)Fc5 c870e6 pZMP31
atggatgcaatgaagagagggctctgctgtgtgctgctgctgtgtggcgccgtcttcgtttcgctcagccaggaaatccatgccgagttgagacgcttccgtagagagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctggccgcaccggcccatcctgcaggcggggctgccggccaaccagacggcggtgctgggcagcgacgtggagttccactgcaaggtgtacagtgacgcacagccccacatccagtggctcaagcacgtggaggtgaatggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcgagcccaaatcttcagacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggtggcgggggttcgggtggaggaggttctgaagttcaattgttagagtctggtggcggtcttgttcagcctggtggttctttacgtctttcttgcgctgcttccggattcactttctctggttacattatgtggtgggttcgccaagctcctggtaaaggtttggagtgggtttctgttatctctccttctggtggcgatacttggtatgctgactccgttaaaggtcgcttcactatctctagagacaactctaagaatactctctacttacagatgaacagcttaagggctgaggacacggccgtgtattactgtgccacagcgggagactactggggccagggcaccctggtcaccgtctcaagcggtggtggtggttctggcggcggcggctccggcgggggtggaagtggtggtggtggttctggtggtggtggttctcagagcgtgttgactcagccaccctcagcgtctgggacccccgggcagagggtcaccatctcttgttctggaagcagctccaacatcggaaggaatcctgtaaactggtaccagcagctcccaggaacggcccccaaactcctcatctatggtgataatcagcggccctcaggggtccctgaccgattctctggctccaaatctggcacctcagcctccctggccatcagtgggctccagtctgaggatgaggctgattattactgtgcagcatgggatgacagcctgaatggtgtggtattcggcggagggaccaagctgaccgtcctataa

SEQ ID NO: 64 - protein, 877 aa, Artificial [sequence omitted]

SEQ ID NO: 65 - DNA, 322 bp, Homo sapien
aacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaac

SEQ ID NO: 66 - protein, 107 aa, Homo sapien
NIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 67 - DNA, 364 bp, Homo sapien
gaagttcaattgttagagtctggtggcggtcttgttcagcctggtggttctttacgtctttcttgcgctgcttccggattcactttctcttggtacgatatggtttgggttcgccaagctcctggtaaaggtttggagtgggtttctgttatctctccttctggtggctggacttcttatgctgactccgttaaaggtcgcttcactatctctagagacaactctaagaatactctctacttgcagatgaacagcttaagggctgaggacacggccgtgtattactgtgcgagagatcatagtggctacgattctgaatactttgactactggggccagggcaccctggtcaccgtctcaagcg

SEQ ID NO: 68 - protein, 121 aa, Homo sapien
EVQLLESGGGLVQPGGSLRLSCAASGFTFSWYDMVWVRQAPGKGLEWVSVISPSGGWTSYADSVKGRFTISRDNSKNTLYLQMNSLRAEDTAVYYCARDHSGYDSEYFDYWGQGTLVTVSS

SEQ ID NO: 69 - DNA, 759 bp, ArtificiaL
  c1039.1 scFv cDNA
gaagttcaattgttagagtctggtggcggtcttgttcagcctggtggttctttacgtctttcttgcgctgcttccggattcactttctcttggtacgatatggtttgggttcgccaagctcctggtaaaggtttggagtgggtttctgttatctctccttctggtggctggacttcttatgctgactccgttaaaggtcgcttcactatctctagagacaactctaagaatactctctacttgcagatgaacagcttaagggctgaggacacggccgtgtattactgtgcgagagatcatagtggctacgattctgaatactttgactactggggccagggcaccctggtcaccgtctcaagcggtggtggtggttctggcggcggcggctccggcgggggtggaagtggtggtggtggttctggtggtggtggttctaacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaa

SEQ ID NO: 70 - protein, 253 aa, Artificial
  c1039.1 scFv
EVQLLESGGGLVQPGGSLRLSCAASGFTFSWYDMVWVRQAPGKGLEWVSVISPSGGWTSYADSVKGRFTISRDNSKNTLYLQMNSLRAEDTAVYYCARDHSGYDSEYFDYWGQGTLVTVSSGGGGSGGGGSGGGGSGGGGSGGGGSNIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 71 - DNA, 990 bp, Homo sapien
  CDS (1)..(990)
cagtgtgctggcggcccggcgcgagccggcccggccccggtcgggcctccgaaaccatgaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacaccgcggccgccagcacactg

SEQ ID NO: 72 - protein, 19 aa, Homo sapien
QCAGGPARAGPAPVGPPKP

SEQ ID NO: 73 - protein, 39 aa, Homo sapien
TFCCLGCIGASPCCSTSTMPSGPRLHPWQKEEGRIITKW

SEQ ID NO: 74 - protein, 38 aa, Homo sapien
SSWMSISAATAIQSRPWWTSSRSTLMRSSTSSSHPVCP

SEQ ID NO: 75 - protein, 36 aa, Homo sapien
CDAGAAAMTRAWSVCPLRSPTSPCRLCGSNLTKAST

SEQ ID NO: 76 - protein, 58 aa, Homo sapien
ASYSTTNVNADQRKIEQDKKIPVGLAQSGESICLYKIRRRVNVPAKTQTRVARRGSLS

SEQ ID NO: 77 - protein, 61 aa, Homo sapien
TNVLADVTSRGGEPGRRKEPPSGFREPDLSPGKTDTERSIQKPRCRHHTITIDRTVLNPET

SEQ ID NO: 78 - protein, 26 aa, Homo sapien
NEGRGDSAQSTLGPEGETPAEAFPGG

SEQ ID NO: 79 - protein, 43 aa, Homo sapien
PSTVPLGIGFAILFFLLLNHRARKIREFYFWDSCRHTAAASTL

SEQ ID NO: 80 - protein, 15 aa, Artificial
  peptide linker
GGGGSGGGGSGGGGS

SEQ ID NO: 81 - protein, 232 aa, Homo sapien
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 232 aa, Homo sapien
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 232 aa, Artificial
  Fc-488
EPRSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 232 aa, Artificial
  Fc-4
EPRSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 231 aa, Artificial
  Fc6
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 232 aa, Artificial
  FC7
EPKSCDKTHTCPPCPAPELLGGPSVFLFPPKPKDTLMISRTPEVTCVVVDVSHEDPEVKFNWYVDGVEVHNAKTKPREEQYESTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 339 aa, Homo sapien
RDESNHLTDLYRRDETIQVKGNGYVQSPRFPNSYPRNLLLTWRLHSQENTRIQLVFDNQFGLEEAENDICRYDFVEVEDISETSTIIRGRWCGHKEVPPRIKSRTNQIKITFKSDDYFVAKPGFKIYYSLLEDFQPAAASETNWESVTSSISGVSYNSPSVTDPTLIADALDKKIAEFDTVEDLLKYFNPESWQEDLENMYLDTPRYRGRSYHDRKSKVDLDRLNDDAKRYSCTPRNYSVNIREELKLANVVFFPRCLLVQRCGGNCGCGTVNWRSCTCNSGKTVKKYHEVLQFEPGHIKRRGRAKTMALVDIQLDHHERCDCICSSRPPRYLEYMPMD

SEQ ID NO: 88 - protein, 1002 aa, Artificial
  VEGFR2-Fc
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSHAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEGSGGSGEPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 48 bp, Artificial
  zc60566
agaacctcctccacccgaacccccgccaccacccggagacagggagag